SEQUENCE LISTING (73 sequences)

SEQ ID NO: 1 - protein, 539 aa, synthetic construct
  REGION 1..539: note = Description of sequence: APHT Alk phos heat tolerant
  REGION 1..539: note = Description of origin: Bacterial derived but modified Artificial Sequence Variant enzyme
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 2 - protein, 539 aa, synthetic construct
  REGION 1..539: note = Description of sequence: AP heat tolerant consensus variants sequence
  REGION 1..539: note = Description of origin: Artificial Sequence Variant enzyme
  VARIANT 33: note = X is: I or L
  VARIANT 67: note = X is: I
  VARIANT 72: note = X is: A
  VARIANT 77: note = X is: I
  VARIANT 84: note = X is: N
  VARIANT 103: note = X is: H or F
  VARIANT 130: note = X is: G
  VARIANT 135: note = X is: N
  VARIANT 165: note = X is: N
  VARIANT 190: note = X is: N
  VARIANT 214: note = X is: P
  VARIANT 257: note = X is: A
  VARIANT 269: note = X is: M V T or E
  VARIANT 271: note = X is: A or E
  VARIANT 305: note = X is: G or A
  VARIANT 502: note = X is: E
ESSNGQSKNLIVLIGDGMGPAQVSAARYFQQHXNNINSLNLDPYYVGQATTYADRGEDGGHIVSGIXTSSAXAGTAXATGNKTXNAAISVSNEDVSRPFASVXEAAELSGKSTGLVTTARITHATPAVYXSHVRXRDNENAIAFQYLDSGIDVLLGGGESFFVTXEEKGKRNDKNLLPEFEAKGYKVVKXGQSLKSLSAKDAKVLGLFGGSHIXYVPDRSDETPSLAEMTSKALEILSTNENGFAIMIEGGRIDHAXHANDFPTMVQEXLXFDEAFKVAIDFAKKDGNTSVVVTADHETGGLSLXRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSXITIDVDNDVLYLPLTAFSQITGQTLKWDALSERIMLK

SEQ ID NO: 3 - protein, 539 aa, synthetic construct
  REGION 1..539: note = Description of sequence: 2051 mutant
  REGION 1..539: note = Description of origin: Artificial Sequence Variant enzyme
ESSNGQSKNLIVLIGDGMGPAQVSAARYFQQHINNINSLNLDPYYVGQATTYADRGEDGGHIVSGIVTSSASAGTALATGNKTYNAAISVSNEDVSRPFASVLEAAELSGKSTGLVTTARITHATPAVYASHVRNRDNENAIAFQYLDSGIDVLLGGGESFFVTNEEKGKRNDKNLLPEFEAKGYKVVKTGQSLKSLSAKDAKVLGLFGGSHIAYVPDRSDETPSLAEMTSKALEILSTNENGFAIMIEGGRIDHAGHANDFPTMVQEALDFDEAFKVAIDFAKKDGNTSVVVTADHETGGLSLSRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSSITIDVDNDVLYLPLTAFSQITGQTLKWDALSERIMLK

SEQ ID NO: 4 - protein, 539 aa, synthetic construct
  REGION 1..539: note = Description of sequence: AP1 mutant single S vs P lentus AP wt
  REGION 1..539: note = Description of origin: Artificial Sequence Variant enzyme
ESSNGQSKNLIVLIGDGMGPAQVSAARYFQQHKNNINSLNLDPYYVGQATTYADRGEDGGHIVSGIVTSSASAGTAFATGNKTYNAAISVSNEDVSRPFASVLEAAELSGKSTGLVTTARITHATPAVYASHVRSRDNENAIAFQYLDSGIDVLLGGGESFFVTKEEKGKRNDKNLLPEFEAKGYKVVKTGQSLKSLSAKDAKVLGLFGGSHIAYVPDRSDETPSLAEMTSKALEILSTNENGFAIMIEGGRIDHAGHANDFPTMVQEALDFDEAFKVAIDFAKKDGNTSVVVTADHETGGLSLSRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSSITIDVDNDVLYLPLTAFSQITGQTLKWDALSERIMLK

SEQ ID NO: 5 - protein, 572 aa, Paenibacillus lentus
  REGION 1..572: note = Description of species/origin: Native AP with leader sequence
MNKLLKGLAIGGIVLAVVSAGTLAVAKENASRAESSNGQSKNLIVLIGDGMGPAQVSAARYFQQHKNNINSLNLDPYYVGQATTYADRGEDGGHIVSGIVTDSASAGTAFATGNKTYNAAISVSNEDVSRPFASVLEAAELSGKSTGLVTTARITHATPAVYASHVRSRDNENAIAFQYLDSGIDVLLGGGESFFVTKEEKGKRNDKNLLPEFEAKGYKVVKTGQSLKSLSAKDAKVLGLFGGSHIAYVPDRSDETPSLAEMTSKALEILSTNENGFAIMIEGGRIDHAGHANDFPTMVQEALDFDEAFKVAIDFAKKDGNTSVVVTADHETGGLSLSRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSSITIDVDNDVLYLPLTAFSQITGQTLKWDALSERIMLK

SEQ ID NO: 6 - protein, 572 aa, synthetic construct
  REGION 1..572: note = Description of sequence: AP1 with D-S mutation with leader sequence
  REGION 1..572: note = Description of origin: Artificial Sequence Variant enzyme
MNKLLKGLAIGGIVLAVVSAGTLAVAKENASRAESSNGQSKNLIVLIGDGMGPAQVSAARYFQQHKNNINSLNLDPYYVGQATTYADRGEDGGHIVSGIVTSSASAGTAFATGNKTYNAAISVSNEDVSRPFASVLEAAELSGKSTGLVTTARITHATPAVYASHVRSRDNENAIAFQYLDSGIDVLLGGGESFFVTKEEKGKRNDKNLLPEFEAKGYKVVKTGQSLKSLSAKDAKVLGLFGGSHIAYVPDRSDETPSLAEMTSKALEILSTNENGFAIMIEGGRIDHAGHANDFPTMVQEALDFDEAFKVAIDFAKKDGNTSVVVTADHETGGLSLSRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSSITIDVDNDVLYLPLTAFSQITGQTLKWDALSERIMLK

SEQ ID NO: 7 - protein, 580 aa, synthetic construct
  REGION 1..580: note = Description of sequence: APHT Amino acid sequence with Hemicell leader
  REGION 1..580: note = Description of origin: Artificial Sequence Variant enzyme
MKNLRKKSLSICMAMAMMFSLVTLLGGQDIRAASGFYVSGTESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 8 - protein, 548 aa, synthetic construct
  REGION 1..548: note = Description of sequence: APHT with leader sequence
  REGION 1..548: note = Description of origin: Artificial Sequence Variant enzyme
ASGFYVSGTESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 9 - DNA, 1716 bp, synthetic construct
  misc_feature 1..1716: note = Description of sequence: Nucleic acid encoding AP1 Seq id 4
  misc_feature 1..1716: note = Description of origin: Artificial Sequence Variant enzyme
  source 1..1716: mol_type = other DNA
gtgaataaattgttgaagggtttggctatcggggggatcgtgcttgcagttgtgtctgctggaacattggctgtggcgaaggagaatgcatctagagctgaaagctcgaacgggcaatcaaaaaatctgatcgttctgatcggggacggtatggggcccgcgcaagtttcggctgcgcgttattttcagcaacacaaaaataatattaacagtctgaacctggatccgtactatgttggacaagcgacgacttatgcagaccgcggcgaagacggaggtcatatcgtctccggaatcgtgacgtcctcagcttccgcgggcaccgcatttgcaacgggcaataaaacatacaatgcggcaataagcgtttccaacgaggatgtatccaggccgttcgcctcggtgcttgaagcagccgagctaagcggcaagtcgacaggactggtaacgacagcccgcattacccatgctactccggcggtatacgcttcacatgtgcgaagccgtgataatgagaacgcaattgctttccagtacttagacagcgggattgacgtattgcttggcggaggggaatccttctttgttacgaaagaggaaaagggcaagcgtaacgataaaaatctgctgccggaattcgaagccaagggatacaaagttgtaaaaacaggtcaaagtctgaagagcctctcggccaaagacgccaaagtgctgggactgtttggaggctctcatatcgcttatgtgccagatcgttcggatgaaactccgagtctcgctgaaatgacttcgaaagctttggaaatcttatccacaaacgagaatggatttgccatcatgattgagggtggacgcatagatcatgctggacacgcgaatgatttccccacgatggtgcaagaagcgctagattttgatgaagcctttaaagtagcgatcgactttgccaagaaggatggaaatacgtcagtcgtcgtcacagctgatcatgagaccggcggcctttccctatcacgggacaacatttacgagttgaacgttgatttgtggaacaagcagaaaaactcctccgagagcctggtatcggctttgaatgaagccaagacgattgcggatgtaaagaaaatcgtgtcggacaatacctggatcactgatcttacgaatgaagaggcacagtatattctagacggtgatggctcttcttacaaacgtgaaggcggttataatgccgtaatctctaagcgtctgctcgtcggctggtctggtcacggacattccgcagtggatgttggagtgtgggcttacggcccaatcgcggataaagtgaaaggacaaatcgacaatacgaggatcgcgacggcaagtgcggaggtgctcggtgttgatctgaaaaaggctacggctgatctccaatcgaagtatctgtatccaaaattcaaaattaaccgcaataaagaagtgctatttccggctaagcctttggctgaggcgctaggcggtaagtaccaggcagcaaacggaacggctacaatcagcggcatgagcgggacaattacagtagatctgaatgcaaagaaggctaaattaagcgggaattcttcctctattaccatagatgttgacaatgatgtgctttatcttccgttaacagcatttagccaaattacaggacaaaccttgaaatgggacgcattgtctgaacggattatgttgaaa

SEQ ID NO: 10 - protein, 4 aa, synthetic construct
  REGION 1..4: note = Description of sequence: Leader sequence
ASRA

SEQ ID NO: 11 - protein, 463 aa, synthetic construct
  REGION 1..463: note = Description of sequence: AP1 with ASRN leader
ASRAESSNGQSKNLIVLIGDGMGPAQVSAARYFQQHKNNINSLNLDPYYVGQATTYADRGEDGGHIVSGIVTSSASAGTAFATGNKTYNAAISVSNEDVSRPFASVLEAAELSGKSTGLVTTARITHATPAVYASHVRSRDNENAIAFQYLDSGIDVLLGGGESFFVTKEEKGKRNDKNLLPEFEAKGYKVVKTGQSLKSLSAKDAKVLGLFGGSHIAYVPDRSDETPSLAEMTSKALEIWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSSITIDVDNDVLYLPLTAFSQITGQTLKWDALSERIMLK

SEQ ID NO: 12 - protein, 463 aa, synthetic construct
  REGION 1..463: note = Description of sequence: 2051 mutant AP with ASRN leader
ASRAESSNGQSKNLIVLIGDGMGPAQVSAARYFQQHINNINSLNLDPYYVGQATTYADRGEDGGHIVSGIVTSSASAGTALATGNKTYNAAISVSNEDVSRPFASVLEAAELSGKSTGLVTTARITHATPAVYASHVRNRDNENAIAFQYLDSGIDVLLGGGESFFVTNEEKGKRNDKNLLPEFEAKGYKVVKTGQSLKSLSAKDAKVLGLFGGSHIAYVPDRSDETPSLAEMTSKALEIWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSSITIDVDNDVLYLPLTAFSQITGQTLKWDALSERIMLK

SEQ ID NO: 13 - protein, 9 aa, synthetic construct
  REGION 1..9: note = Description of sequence: Leader sequence
ASGFYVSGT

SEQ ID NO: 14 - protein, 548 aa, synthetic construct
  REGION 1..548: note = Description of sequence: AP1 with leader sequence ASGFYVSGT
ASGFYVSGTESSNGQSKNLIVLIGDGMGPAQVSAARYFQQHKNNINSLNLDPYYVGQATTYADRGEDGGHIVSGIVTSSASAGTAFATGNKTYNAAISVSNEDVSRPFASVLEAAELSGKSTGLVTTARITHATPAVYASHVRSRDNENAIAFQYLDSGIDVLLGGGESFFVTKEEKGKRNDKNLLPEFEAKGYKVVKTGQSLKSLSAKDAKVLGLFGGSHIAYVPDRSDETPSLAEMTSKALEILSTNENGFAIMIEGGRIDHAGHANDFPTMVQEALDFDEAFKVAIDFAKKDGNTSVVVTADHETGGLSLSRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSSITIDVDNDVLYLPLTAFSQITGQTLKWDALSERIMLK

SEQ ID NO: 15 - protein, 548 aa, synthetic construct; identical to SEQ ID NO: 8
  REGION 1..548: note = Description of sequence: APHT polypeptide with leader sequence ASGFYVSGT

SEQ ID NO: 16 - protein, 33 aa, Paenibacillus lentus
  REGION 1..33: note = Description of species/origin: Native Leader sequence
MNKLLKGLAIGGIVLAVVSAGTLAVAKENASRA

SEQ ID NO: 17 - protein, 41 aa, Paenibacillus lentus
  REGION 1..41: note = Description of species/origin: Native Hemicell leader sequence
MKNLRKKSLSICMAMAMMFSLVTLLGGQDIRAASGFYVSGT

SEQ ID NO: 18 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 19 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 20 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 21 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 22 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 23 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 24 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 25 - protein, 539 aa, synthetic construct; identical to SEQ ID NO: 18
  REGION 1..539: note = Description of sequence: AP heat tolerant variant

SEQ ID NO: 26 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 27 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 28 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 29 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 30 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 31 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 32 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 33 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 34 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 35 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 36 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 37 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 38 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 39 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 40 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 41 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 42 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 43 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 44 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 45 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 46 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 47 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 48 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 49 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 50 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 51 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 52 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 53 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVHEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 54 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 55 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 56 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 57 - protein, 539 aa, synthetic construct
  REGION 1..539: note = Description of sequence: AP heat tolerant variant
ESSNGQSKNLIVLIGDGMGPAQVSAARYFQQHINNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 58 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 59 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 60 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 61 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELAFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 62 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 63 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 64 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 65 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELEFDEAFKVAIDFAKKDGNTSVVVTADHETGGLSLGRDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 66 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 67 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 68 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 69 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELEFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 70 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEMLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 71 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEVLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 72 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQETLAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

SEQ ID NO: 73 - protein, 539 aa, synthetic construct
  REGION 1..539: note = Description of sequence: AP heat tolerant variant
ESSNGQSKNLIVLIGDGMGPAQVSAARYFQQHLNNINSLNLDPYYVGQATTYADRGEDGGHIVSGIITSSAAAGTAIATGNKTNNAAISVSNEDVSRPFASVFEAAELSGKSTGLVTTARITHATPAVYGSHVRNRDNENAIAFQYLDSGIDVLLGGGESFFVTNEEKGKRNDKNLLPEFEAKGYKVVKNGQSLKSLSAKDAKVLGLFGGSHIPYVPDRSDETPSLAEMTSKALEILSTNENGFAIMIEGGRIDHAAHANDFPTMVQEELAFDEAFKVAIDFAKKDGNTSVVVTADHETGGLSLARDNIYELNVDLWNKQKNSSESLVSALNEAKTIADVKKIVSDNTWITDLTNEEAQYILDGDGSSYKREGGYNAVISKRLLVGWSGHGHSAVDVGVWAYGPIADKVKGQIDNTRIATASAEVLGVDLKKATADLQSKYLYPKFKINRNKEVLFPAKPLAEALGGKYQAANGTATISGMSGTITVDLNAKKAKLSGNSSEITIDVDNDVLYLPLTAFSQITGQTLKWDALSERIMLK

(19) United States
(12) Patent Application Publication
(10) Pub. No.: US 2025/0011738 A1
(43) Pub. Date: Jan. 9, 2025

(54) HEAT TOLERANT ALKALINE PHOSPHATASE

(71) Applicant: BiomEdit, LLC, Fishers, IN (US)

(72) Inventors: Lin Liu, Fishers, IN (US); Jeffery Escobar, Fishers, IN (US)

(21) Appl. No.: 18/712,348

(22) PCT Filed: Nov. 30, 2022
(86) PCT No.: PCT/US2022/051325

§ 371 Date: May 22, 2024

Related U.S. Application Data
(60) Provisional application No. 63/284,530, filed on Nov. 30, 2021.

(51) Int. Cl. C12N 9/16; A23K 20/189; A23K 50/30; A23K 50/60; A23K 50/75

(52) U.S. Cl. CPC C12N 9/16; A23K 20/189; A23K 50/30; A23K 50/60; A23K 50/75; C12Y 301/03001

(57) ABSTRACT

The present invention provides alkaline phosphatase enzyme peptides, particularly microbial alkaline phosphatase enzyme peptides, that are mutant peptides altered in sequence and having increased heat tolerance while retaining enzymatic activity, and their use and application including in animal feed and for beneficial health and growth effects in animals.

### CROSS REFERENCE TO RELATED APPLICATIONS

The present application claims priority to U.S. Application Ser. No. 63/284,530, filed Nov. 30, 2021, the entire contents of which is incorporated by reference herein.

### SEQUENCE LISTING

This application contains a Sequence Listing, which was submitted as an electronic document in XML format via EFS-Web, and is hereby incorporated by reference in its entirety. The XML copy, created on Nov. 21, 2022, is entitled “2950-15PCT_ST26.xml” and is 321,106 bytes in size.

### FIELD OF THE INVENTION

The present invention relates to alkaline phosphatase enzyme peptides, particularly microbial alkaline phosphatase enzyme peptides, that are mutant peptides altered in sequence and having increased heat tolerance while retaining enzymatic activity and their use and application including in animal feed, feed additives, and for beneficial health and growth effects in animals.

### BACKGROUND OF THE INVENTION

Enzymes, including lysozyme and alkaline phosphatase, are recognized as disruptors of bacterial debris and inflammation. These enzymes reduce inflammation, including by lysing the peptidoglycans and lipopolysaccharides (LPS) of bacteria, thus deactivating them and thereby bypassing or eliminating the inflammatory response. Alkaline phosphatase (AP) is a complex enzyme that is a homodimer with three metal ions in the active site that participate in the catalysis. The AP structure and mechanism from several sources has been studied extensively, including work on the bacterial alkaline phosphatase from E. coli. AP is also used widely as a reagent in biomedical diagnostic kits, is measured directly during blood chemistry analysis for health monitoring, and AP levels are also measured during the pasteurization of milk. Thus, there are a wide variety of substrates and assays that have been developed to work with and assess this enzyme (Gonzalez-Gil, S et al (1998) Marine Ecology Progress Series 164: 21-35). The fact that the enzyme has relatively non-specific activity hydrolyzing phosphate monoesters creates many assay opportunities using numerous colorimetric and fluorescent substrates. The involvement of both Zn⁺⁺ and Mg⁺⁺ ions in the structure and function can result in complex metal ion inhibition/activation patterns (Dealwis, C G et al (1995) Biochemistry 34 (43): 13967-13973; Davidson, DF (1979) Enzyme Microb Technol 1: 9-14).

Alkaline phosphatases (AP) are homodimeric enzymes that catalyze the hydrolysis of monoesters of phosphoric acid and transphosphorylation reactions. AP are naturally occurring in the mammalian body and are divided into four types: tissue non-specific AP (TNAP), placental AP (PLAP), germ cell AP (GCAP) and intestinal AP (IAP). Intestinal alkaline phosphatase (IAP) is a glycoprotein anchored in the apical membrane of the small intestine and is found in the highest levels in the duodenum, followed by the jejunum and ileum. IAP has several biological roles, including being a negative regulator of intestinal fat absorption, maintaining bicarbonate secretion and pH balance, and exerting immune-protective effects. Expression of IAP is dependent upon enterocyte differentiation; therefore, the enzyme is often used as a biomarker for abnormal digestive and absorptive functions in the small intestine. A significant role of alkaline phosphatase in vivo is to detoxify bacterial LPS that is present in the intestinal lumen.

Many studies evaluating the benefits of alkaline phosphatase utilize the mammalian IAP isoform which is often derived from swine or bovine. Obtaining IAP from a mammalian host requires euthanizing the animal and scraping the mucosa of the small intestine, followed by various digestion and isolation techniques. This process requires land and money to house the animals and feed for them, produces negative environmental output, and has enzymatic variability between animals due to diet, disease state, and genetics. Furthermore, it requires large numbers of animals for a small amount of enzyme. Overall, it results in a significant economic and environmental cost for a highly variable product.

Microbial enzymes are used in a variety of fields and have a large number of biotechnological applications. Bacterial hosts can be engineered to rapidly and efficiently overexpress recombinant enzymes and then cultured in large quantities to produce a substantial amount of the desired enzymes (Liu L et al (2013) Bioengineered 4(4):212-223; doi:10.4161/bioe.24761). The fast growth rate and simple requirements of microbes make them a more sustainable, economically, and environmentally friendly option. Microbial-derived enzymes are replacing conventional enzymatic production methods due to their consistency, ease of optimization, regular supply, and greater catalytic activity.

Alkaline Phosphatase, including provided as a feed enzyme, has the potential to be a unique and useful product for poultry and swine especially in light of the roles of AP in the digestive tract. Production of microbial alkaline phosphatase compositions, including feed or feed additives, requires the availability of an active and effective enzyme polypeptide which is heat tolerant and can retain its activity and effectiveness through the manufacturing process, including for example feed pelleting or additive preparation processes. There is a need for a heat tolerant and active AP enzyme polypeptide for any of various uses, compositions, applications and methods.

The citation of references herein shall not be construed as an admission that such is prior art to the present invention.

### SUMMARY OF THE INVENTION

The present invention relates to mutant alkaline phosphatase enzyme peptides having heat tolerance and retaining activity with incubation at increased temperatures for long periods of time as candidates and peptides for various uses and applications. In a particular aspect, the mutant alkaline phosphatase enzyme peptides are variants or mutants of the Paenibacillus lentus bacteria alkaline phosphatase enzyme. Mutant alkaline phosphatase enzyme peptides comprise one or more variant, altered, or modified amino acid that renders the peptides heat tolerant. Mutant alkaline phosphatase enzyme peptides comprise one or more variant, altered, or modified amino acid that renders the peptides at least 10° C. temperature improvement in thermostability. In an embodiment, the mutant alkaline phosphatase enzyme peptides retain phosphatase activity which is not significantly different from native AP enzyme phosphatase activity after treatment or incubation at up to 80° C., up to 85° C., up to 90° C., up to 93° C., up to 95° C. In an embodiment, the mutant alkaline phosphatase enzyme peptides retain phosphatase activity which is not significantly different from native AP enzyme phosphatase activity after treatment or incubation at up to 80° C., up to 85° C., up to 90° C., up to 93° C., up to 95° C. for at least 5 minutes, at least 10 minutes, at least 15 minutes, up to 10 minutes, up to 15 minutes, up to 20 minutes. The mutant alkaline phosphatase peptides have improved and useful characteristics to provide greater utility and application, including as a feed additive and in animals, including in animal health.

In an embodiment, a heat tolerant bacterial alkaline phosphatase enzyme polypeptide is provided, said polypeptide comprising:
(a) the polypeptide

(SEQ ID NO: 2)
ESSNGQSKNLIVLIGDGMGPAQVSAARYFQQHX1NNINSLNLDPYYVGQA
TTYADRGEDGGHIVSGIX2TSSAX3AGTAX4ATGNKTX5NAAISVSNEDVS
RPFASVX6EAAELSGKSTGLVTTARITHATPAVYX7SHVRX8RDNENAIAF
QYLDSGIDVLLGGGESFFVTX9EEKGKRNDKNLLPEFEAKGYKVVKX10GQ
SLKSLSAKDAKVLGLFGGSHIX11YVPDRSDETPSLAEMTSKALEILSTNE
NGFAIMIEGGRIDHAX12HANDFPTMVQEX13LX14FDEAFKVAIDFAKKDG
NTSVVVTADHETGGLSLX15RDNIYELNVDLWNKQKNSSESLVSALNEAK
TIADVKKIVSDNTWITDLTNEEAQYILDGDGSSYKREGGYNAVISKRLL
VGWSGHGHSAVDVGVWAYGPIADKVKGQIDNTRIATASAEVLGVDLKKA
TADLQSKYLYPKFKINRNKEVLFPAKPLAEALGGKYQAANGTATISGMS
GTITVDLNAKKAKLSGNSSX16ITIDVDNDVLYLPLTAFSQITGQTLKWD
ALSERIMLK

wherein:
   X₁ is I or L,
   X₂ is I,
   X₃ is A,
   X₄ is I,
   X₅ is N,
   X₆ is H or F,
   X₇ is G,
   X₈ is N,
   X₉ is N,
   X₁₀ is N.
   X₁₁ is P,
   X₁₂ is A,
   X₁₃ is M, V, T or E,
   X₁₄ is A or E,
   X₁₅ is G or A, and
   X₁₆ is E;
(b) the polypeptide:

(SEQ ID NO: 3)
ESSNGQSKNLIVLIGDGMGPAQVSAARYFQQHINNINSLNLDPYYVGQA
TTYADRGEDGGHIVSGIVTSSASAGTALATGNKTYNAAISVSNEDVSRP
FASVLEAAELSGKSTGLVTTARITHATPAVYASHVRNRDNENAIAFQYL
DSGIDVLLGGGESFFVTNEEKGKRNDKNLLPEFEAKGYKVVKTGQSLKS
LSAKDAKVLGLFGGSHIAYVPDRSDETPSLAEMTSKALEILSTNENGFA
IMIEGGRIDHAGHANDFPTMVQEALDFDEAFKVAIDFAKKDGNTSVVVT
ADHETGGLSLSRDNIYELNVDLWNKQKNSSESLVSALNEAKTIADVKKI
VSDNTWITDLTNEEAQYILDGDGSSYKREGGYNAVISKRLLVGWSGHGH
SAVDVGVWAYGPIADKVKGQIDNTRIATASAEVLGVDLKKATADLQSKY
LYPKFKINRNKEVLFPAKPLAEALGGKYQAANGTATISGMSGTITVDLN
AKKAKLSGNSSSITIDVDNDVLYLPLTAFSQITGQTLKWDALSERIMLK;
or

(c) a variant of (a) or (b), wherein the variant of the alkaline phosphatase enzyme polypeptides retains phosphatase activity which is not significantly different from heat tolerant mutant enzyme SEQ ID NO:1.

In an embodiment, a heat tolerant bacterial alkaline phosphatase enzyme polypeptide is provided, denoted APHT, said polypeptide comprising:

(SEQ ID NO: 1)
ESSNGQSKNLIVLIGDGMGPAQVSAARYFQQHINNINSLNLDPYYVGQA
TTYADRGEDGGHIVSGIITSSAAAGTAIATGNKTNNAAISVSNEDVSRP
FASVHEAAELSGKSTGLVTTARITHATPAVYGSHVRNRDNENAIAFQYL
DSGIDVLLGGGESFFVTNEEKGKRNDKNLLPEFEAKGYKVVKNGQSLKS
LSAKDAKVLGLFGGSHIPYVPDRSDETPSLAEMISKALEILSTNENGFA
IMIEGGRIDHAAHANDFPTMVQEMLAFDEAFKVAIDFAKKDGNTSVVVT
ADHETGGLSLGRDNIYELNVDLWNKQKNSSESLVSALNEAKTIADVKKI
VSDNTWITDLINEEAQYILDGDGSSYKREGGYNAVISKRLLVGWSGHGH
SAVDVGVWAYGPIADKVKGQIDNTRIATASAEVLGVDLKKATADLQSKY
LYPKFKINRNKEVLFPAKPLAEALGGKYQAANGTATISGMSGTITVDLN
AKKAKLSGNSSEITIDVDNDVLYLPLTAFSQITGQTLKWDALSERIMLK.

In an embodiment, a heat tolerant bacterial alkaline phosphatase enzyme polypeptide is provided, said polypeptide comprising:

(SEQ ID NO: 3)
ESSNGQSKNLIVLIGDGMGPAQVSAARYFQQHINNINSLNLDPYYVGQA
TTYADRGEDGGHIVSGIVTSSASAGTALATGNKTYNAAISVSNEDVSRP
FASVLEAAELSGKSTGLVTTARITHATPAVYASHVRNRDNENAIAFQYL
DSGIDVLLGGGESFFVTNEEKGKRNDKNLLPEFEAKGYKVVKTGQSLKS
LSAKDAKVLGLFGGSHIAYVPDRSDETPSLAEMTSKALEILSTNENGFA
IMIEGGRIDHAGHANDFPTMVQEALDFDEAFKVAIDFAKKDGNTSVVVT
ADHETGGLSLSRDNIYELNVDLWNKQKNSSESLVSALNEAKTIADVKKI
VSDNTWITDLTNEEAQYILDGDGSSYKREGGYNAVISKRLLVGWSGHGH
SAVDVGVWAYGPIADKVKGQIDNTRIATASAEVLGVDLKKATADLQSKY
LYPKFKINRNKEVLFPAKPLAEALGGKYQAANGTATISGMSGTITVDLN
AKKAKLSGNSSSITIDVDNDVLYLPLTAFSQITGQTLKWDALSERIMLK.

In other embodiments, a heat tolerant bacterial alkaline phosphatase enzyme polypeptide is provided, wherein said polypeptide comprises the polypeptide of any of SEQ ID NOs: 18-73.

In some embodiments, the heat tolerant alkaline phosphatase enzyme polypeptide further comprises an N terminal leader sequence.

In some embodiments, the N terminal leader sequence is a sequence of 2 to 10 amino acids and wherein the leader sequence does not alter the heat tolerance or the enzymatic activity of the phosphatase.

In some embodiments, the N terminal leader sequence is selected from SEQ ID NO: 10, 13, 16 and 17.

In some embodiments, variants of the heat tolerant bacterial alkaline phosphatase enzyme polypeptide are included and provided wherein one or more additional amino acid is changed or substituted. In some embodiments, variants of the heat tolerant bacterial alkaline phosphatase enzyme polypeptide are included and provided wherein one or more, one to six, at least one, one or two, one or up to three, one or up to 4, one or up to 5, one or up to 6, one or up to 7, one or up to 8, one or up to 9, one or up to 10 additional amino acid is changed or substituted. In embodiments, the variant(s) retain heat tolerance. In embodiments, the variants retain heat tolerance or are heat tolerant. In an embodiment, the variants of the mutant alkaline phosphatase enzyme peptides retain phosphatase activity which is not significantly different from native AP enzyme phosphatase activity after treatment or incubation at up to 80° C., up to 85° C., up to 90° C., up to 93° C., up to 95° C. for at least 5 minutes, at least 10 minutes, at least 15 minutes, up to 10 minutes, up to 15 minutes, up to 20 minutes. In an embodiment, the additional variants of the mutant alkaline phosphatase enzyme peptides retain phosphatase activity which is not significantly different from heat tolerant mutant enzyme SEQ ID NO:1 phosphatase activity after treatment or incubation at up to 80° C., up to 85° C., up to 90° C., up to 93° C., up to 95° C. for at least 5 minutes, at least 10 minutes, at least 15 minutes, up to 10 minutes, up to 15 minutes, up to 20 minutes. In some embodiments, the variant does not correspond in sequence to SEQ ID NO:4. In some embodiments, the variants are 95%, 96%, 97%, 98% or 99% identical in amino acid sequence to SEQ ID NO: 1, 2 or 3. In some embodiments, the variants are 95%, 96%, 97%, 98% or 99% identical in amino acid sequence to SEQ ID NO: 1, 2 or 3 and do not correspond in sequence to SEQ ID NO:4.

In embodiments, a composition, particularly a feed additive or feed composition is provided herein. The feed additive or feed composition comprises one or more mutant heat tolerant alkaline phosphatase as provided herein. The feed additive or feed composition may comprise one or more mutant heat tolerant alkaline phosphatase, or a heat tolerant variant thereof, as provided herein.

In some embodiments, the feed additive or feed composition comprises the mutant alkaline phosphatase enzyme polypeptide of SEQ ID NO: 1, 2, 3 or 18-73. In some embodiments, the feed additive or feed composition comprises the mutant alkaline phosphatase enzyme polypeptide of SEQ ID NO: 2. In some embodiments, the feed additive or feed composition comprises the mutant alkaline phosphatase enzyme polypeptide of SEQ ID NO: 1.

In embodiments, the feed additive or feed composition may comprise the mutant alkaline phosphatase enzyme polypeptide of SEQ ID NO: 1, 2, 3 or 18-73 in an amount of 4,000 IU/kg, 10,000 IU/kg, 12,000 IU/kg, 20,000 IU/kg, 40,000 IU/kg, 100,000 IU/kg, up to 200,000 IU/kg. In some embodiments, the feed additive or feed composition may comprise the mutant alkaline phosphatase enzyme polypeptide of SEQ ID NO: 1, 2, 3 or 18-73 in an amount of 4,000 IU/kg, 10,000 IU/kg, 20,000 IU/kg, up to 40,000 IU/kg.

In an embodiment, the invention provides a mutant alkaline phosphatase enzyme, denoted APHT, said polypeptide comprising:

(SEQ ID NO: 1)
ESSNGQSKNLIVLIGDGMGPAQVSAARYFQQHINNINSLNLDPYYVGQA
TTYADRGEDGGHIVSGIITSSAAAGTAIATGNKTNNAAISVSNEDVSRP
FASVHEAAELSGKSTGLVTTARITHATPAVYGSHVRNRDNENAIAFQYL
DSGIDVLLGGGESFFVTNEEKGKRNDKNLLPEFEAKGYKVVKNGQSLKS
LSAKDAKVLGLFGGSHIPYVPDRSDETPSLAEMTSKALEILSTNENGFA
IMIEGGRIDHAAHANDFPTMVQEMLAFDEAFKVAIDFAKKDGNTSVVVT
ADHETGGLSLGRDNIYELNVDLWNKQKNSSESLVSALNEAKTIADVKKI
VSDNTWITDLINEEAQYILDGDGSSYKREGGYNAVISKRLLVGWSGHGH
SAVDVGVWAYGPIADKVKGQIDNTRIATASAEVLGVDLKKATADLQSKY
LYPKFKINRNKEVLFPAKPLAEALGGKYQAANGTATISGMSGTITVDLN
AKKAKLSGNSSEITIDVDNDVLYLPLTAFSQITGQTLKWDALSERIMLK

In an embodiment, the invention provides a mutant alkaline phosphatase enzyme which is heat tolerant said polypeptide comprising:

(SEQ ID NO: 2)
ESSNGQSKNLIVLIGDGMGPAQVSAARYFQQHX1NNINSLNLDPYYVGQ
ATTYADRGEDGGHIVSGIX2TSSAX3AGTAX4ATGNKTX5NAAISVSNED
VSRPFASVX6EAAELSGKSTGLVTTARITHATPAVYX7SHVRX8RDNENA
IAFQYLDSGIDVLLGGGESFFVTX9EEKGKRNDKNLLPEFEAKGYKVVK
X10GQSLKSLSAKDAKVLGLFGGSHIX11YVPDRSDETPSLAEMTSKALE
ILSTNENGFAIMIEGGRIDHAX12HANDFPTMVQEX13LX14FDEAFKVA
IDFAKKDGNTSVVVTADHETGGLSLX15RDNIYELNVDLWNKQKNSSES
LVSALNEAKTIADVKKIVSDNTWITDLTNEEAQYILDGDGSSYKREGG
YNAVISKRLLVGWSGHGHSAVDVGVWAYGPIADKVKGQIDNTRIATAS
AEVLGVDLKKATADLQSKYLYPKFKINRNKEVLFPAKPLAEALGGKYQ
AANGTATISGMSGTITVDLNAKKAKLSGNSSX16ITIDVDNDVLYLPLT
AFSQITGQTLKWDALSERIMLK

wherein:
X₁ is I or L,
X₂ is I,
X₃ is A,
X₄ is I,
X₅ is N,
X₆ is H or F,
X₇ is G,
X₈ is N,
X₉ is N,
X₁₀ is N.
X₁₁ is P,
X₁₂ is A,
X₁₃ is M, V, T or E,
X₁₄ is A or E,
X₁₅ is G or A, and
X₁₆ is E.

In an embodiment, the invention provides a mutant alkaline phosphatase enzyme, denoted 2051, said polypeptide comprising:

(SEQ ID NO: 3)
ESSNGQSKNLIVLIGDGMGPAQVSAARYFQQHINNINSLNLDPYYVGQATTYADRGEDGGHIVS
GIVTSSASAGTALATGNKTYNAAISVSNEDVSRPFASVLEAAELSGKSTGLVTTARITHATPAVY
ASHVRNRDNENAIAFQYLDSGIDVLLGGGESFFVTNEEKGKRNDKNLLPEFEAKGYKVVKTGQS
LKSLSAKDAKVLGLFGGSHIAYVPDRSDETPSLAEMTSKALEILSTNENGFAIMIEGGRIDHAGHA
NDFPTMVQEALDFDEAFKVAIDFAKKDGNTSVVVTADHETGGLSLSRDNIYELNVDLWNKQKN
SSESLVSALNEAKTIADVKKIVSDNTWITDLTNEEAQYILDGDGSSYKREGGYNAVISKRLLVGW
SGHGHSAVDVGVWAYGPIADKVKGQIDNTRIATASAEVLGVDLKKATADLQSKYLYPKFKINR
NKEVLFPAKPLAEALGGKYQAANGTATISGMSGTITVDLNAKKAKLSGNSSSITIDVDNDVLYLP
LTAFSQITGQTLKWDALSERIMLK

In an embodiment, the invention provides a mutant alkaline phosphatase enzyme of SEQ ID NO:2. In an embodiment, the invention provides a mutant alkaline phosphatase enzyme of SEQ ID NO:2, wherein the mutant comprises or consists of an enzyme amino acid sequence set out in any of SEQ ID NOs: 18-73. In an embodiment, the invention provides a mutant alkaline phosphatase enzyme set out in any of SEQ ID NOs: 18-73. In an embodiment, the invention provides a mutant alkaline phosphatase enzyme set out in any of SEQ ID NOs: 1, 3 or 18-73.

In an embodiment, the mutant alkaline phosphatase enzyme polypeptide includes leader sequence or N terminal additional amino acids. In an embodiment, the leader sequence or N terminal additional amino acids are of a length of 2 to 15, 2 to 10, about 5-7, about 4-8 amino acids. In an embodiment, leader sequence or N terminal additional amino acids do not alter enzyme activity or affect (to increase or decrease) the heat tolerance of the mutant alkaline phosphatase enzyme polypeptide. In an embodiment, leader sequence or N terminal additional amino acids provide increased expression or solubility or stability of the alkaline phosphatase enzyme. Exemplary and utilized leader peptide sequences provided herein include ASRA (SEQ ID NO:10) and ASGFYVSGT (SEQ ID NO:13).

In an embodiment, the mutant alkaline phosphatase enzyme polypeptide is initially synthesized with a longer N-terminal additional peptide sequence, which can then be processed to leave a shorter leader peptide remaining. In some embodiments herein, exemplary longer N-terminal additional peptide sequences include leaders SEQ ID NO:16 and SEQ ID NO:17. These longer N-terminal additional peptide sequence leaders are processed to provide shorter remaining leader peptides N-terminally to the mutant alkaline phosphatase enzyme polypeptide, such as SEQ ID NO: 10 and 13.

In an embodiment, the mutant alkaline phosphatase enzyme polypeptide comprises the amino acid sequence set out in SEQ ID NO: 1, 2, 3, or 18-73 and further comprises N terminal leader or additional N terminal amino acid sequences which do not alter or significantly reduce its activity. In an embodiment, the mutant alkaline phosphatase enzyme polypeptide comprises the amino acid sequence set out in SEQ ID NO: 1 or 2 and further comprises N terminal leader or additional N terminal amino acid sequences which do not alter or significantly reduce its activity. In some embodiments, the N terminal leader or additional N terminal amino acid sequences can improve the activity, stability or expression of the mutant alkaline phosphatase enzyme polypeptide. Exemplary mutant enzymes including such N terminal leader or additional N terminal amino acid sequences include SEQ ID NOs: 7, 8, 12 and 15.

Additional variants of the mutant alkaline phosphatase enzyme polypeptides detailed herein are included as embodiments, particularly wherein the variants retain heat tolerance or are heat tolerant. In an embodiment, the additional variants of the mutant alkaline phosphatase enzyme peptides retain phosphatase activity which is not significantly different from native AP enzyme phosphatase activity after treatment or incubation at up to 80° C., up to 85° C., up to 90° C., up to 93° C., up to 95° C. for at least 5 minutes, at least 10 minutes, at least 15 minutes, up to 10 minutes, up to 15 minutes, up to 20 minutes. In an embodiment, the additional variants of the mutant alkaline phosphatase enzyme peptides retain phosphatase activity which is not significantly different from heat tolerant mutant enzyme SEQ ID NO:1 phosphatase activity after treatment or incubation at up to 80° C., up to 85° C., up to 90° C., up to 93° C., up to 95° C. for at least 5 minutes, at least 10 minutes, at least 15 minutes, up to 10 minutes, up to 15 minutes, up to 20 minutes. In some embodiments, the variants retain at least 4, at least 5, at least 6, at least 7, at least 8, at least 9, at least 10, at least 11, at least 12, at least 13, at least 14 amino acid changes set out in SEQ ID NO:2 versus wild type AP enzyme or non heat tolerant AP enzyme, such as set out in SEQ ID NO:4. In some embodiments, the variants retain at least 4, at least 6, at least 8, at least 10, at least 12, at least 14 amino acid changes set out in SEQ ID NO:2 versus wild type AP enzyme or non heat tolerant AP enzyme, such as set out in SEQ ID NO:4. In some embodiments, the variants retain at least 4, at least 6, at least 8, up to 16 amino acid changes set out in SEQ ID NO:2 versus wild type AP enzyme or non heat tolerant AP enzyme, such as set out in SEQ ID NO:4. In some embodiments, the variants retain up to 16 amino acid changes set out in SEQ ID NO:2 versus wild type AP enzyme or non heat tolerant AP enzyme, such as set out in SEQ ID NO:4, and further include up to 2, up to 3 or up to 4 amino acid changes. In some embodiments, the variants retain up to 16 amino acid changes set out in SEQ ID NO:2 versus wild type AP enzyme or non heat tolerant AP enzyme, such as set out in SEQ ID NO:4, and further include 1, 2, or 3 amino acid changes. In some embodiments, the variants retain at least 4, at least 6, at least 8, up to 16 amino acid changes set out in SEQ ID NO:2 versus wild type AP enzyme or non heat tolerant AP enzyme, such as set out in SEQ ID NO:4, and further include 1, 2, or 3 additional amino acid changes. In some embodiments, the variants retain the amino acid sequences changes and the sequence corresponding to that set out in SEQ ID NO:2, and further include 1, 2, or 3 additional amino acid changes. In some embodiments, the variants retain the amino acid sequences changes and the sequence corresponding to that set out in SEQ ID NO:2, and further include 1, 2, or 3 additional amino acid changes and retain heat tolerance. In all such instances and embodiments, the variant(s) retain heat tolerance, retaining phosphatase activity which is not significantly different from heat tolerant mutant enzyme SEQ ID NO:1 phosphatase activity after treatment or incubation at up to 80° C., up to 85° C., up to 90° C., up to 93° C., up to 95° C. for at least 5 minutes, at least 10 minutes, at least 15 minutes, up to 10 minutes, up to 15 minutes, up to 20 minutes.

In embodiments, a composition is provided comprising one or more mutant alkaline phosphatase enzyme polypeptide described herein and an additive or diluent or carrier.

In some embodiments, the composition further comprises an anti-inflammatory agent, molecule or cytokine. In some embodiments, the composition further comprises an anti-bacterial and/or an anti-infective agent. An anti-bacterial agent may include an antibiotic. An anti-infective agent may include a vaccine or antigen.

The present invention provides enzymes, compositions and methods for improving animal health. The enzymes, compositions and methods improve the health of an animal, including in reducing bacterial lipopolysaccharides (LPS) in the intestine, reducing inflammation particularly bacterial-mediated inflammation, reducing production of pro-inflammatory cytokines, particularly in the intestine, improving intestinal permeability markers, improving performance parameters, and improving or resulting in more efficient nutrient utilization in an animal. In some embodiments, the enzymes, compositions and methods alleviate the effects of chronic alcohol consumption, leaky gut, increased intestinal permeability and inflammation and treat or prevent alcohol-associated intestinal dysbiosis, leaky gut, increased intestinal permeability and inflammation.

In one embodiment, the invention provides a composition comprising one or more mutant heat tolerant alkaline phosphatase, wherein said composition increases animal health when an effective amount is administered to an animal, as compared to an animal not administered the composition. In one embodiment, the invention provides a composition having at least one of a mutant heat tolerant alkaline phosphatase of SEQ ID NO: 1, 2, 3 or 18-73, or a heat tolerant variant thereof, wherein said composition increases animal health when an effective amount is administered to an animal, as compared to an animal not administered the composition. In one embodiment, the invention provides a composition having at least one of a mutant heat tolerant alkaline phosphatase of SEQ ID NO: 1, 2, 3, 7, 8, 12, 15 or 18-73, or a heat tolerant variant thereof, wherein said composition increases animal health when an effective amount is administered to an animal, as compared to an animal not administered the composition.

In one embodiment, the invention provides a composition having at least one of a mutant heat tolerant alkaline phosphatase of SEQ ID NO: 1, 2, 3 or 18-73, or a heat tolerant variant thereof, wherein said composition increases animal health, including in reducing bacterial lipopolysaccharides (LPS) in the intestine, reducing inflammation particularly bacterial-mediated inflammation, reducing production of pro-inflammatory cytokines, particularly in the intestine, improving intestinal permeability markers, improving performance parameters, and improving or resulting in more efficient nutrient utilization in an animal, when an effective amount is administered to an animal, as compared to an animal not administered the composition. In one embodiment, the invention provides a composition having at least one of a mutant heat tolerant alkaline phosphatase of SEQ ID NO: 1, 2, 3 or 18-73, or a heat tolerant variant thereof, wherein said composition increases animal health, including in alleviating the effects of chronic alcohol consumption, leaky gut, increased intestinal permeability and inflammation, when an effective amount is administered to an animal, as compared to an animal not administered the composition. In one embodiment, the invention provides a composition comprising the mutant heat tolerant alkaline phosphatase of SEQ ID NO: 1, or a heat tolerant variant thereof, wherein said composition increases animal health, including in alleviating the effects of chronic alcohol consumption, leaky gut, increased intestinal permeability and inflammation, when an effective amount is administered to an animal, as compared to an animal not administered the composition. In one embodiment, the invention provides a composition comprising the mutant heat tolerant alkaline phosphatase of SEQ ID NO: 2, or a heat tolerant variant thereof, wherein said composition increases animal health, including in alleviating the effects of chronic alcohol consumption, leaky gut, increased intestinal permeability and inflammation, when an effective amount is administered to an animal, as compared to an animal not administered the composition. In one embodiment, the invention provides a composition comprising the mutant heat tolerant alkaline phosphatase of SEQ ID NO: 1, wherein said composition increases animal health, including in alleviating the effects of chronic alcohol consumption, leaky gut, increased intestinal permeability and inflammation, when an effective amount is administered to an animal, as compared to an animal not administered the composition.

The disclosure provides methods for reducing bacterial lipopolysaccharides (LPS) in the intestine, reducing inflammation particularly bacterial-mediated inflammation, reducing production of pro-inflammatory cytokines, particularly in the intestine, improving intestinal permeability markers, improving performance parameters, and improving or resulting in more efficient nutrient utilization in an animal, all and any of which include administration of an effective amount of at least one of a mutant heat tolerant alkaline phosphatase as described herein. The invention provides methods for reducing intestinal permeability, alleviating alcohol induced or disease related leaky gut syndrome, alleviating the intestinal and systemic effects of chronic alcohol consumption, including intestinal dysbiosis, and reducing inflammation, including intestinal-derived or intestinal-associated inflammation, all and any of which include administration of an effective amount of at least one of a mutant heat tolerant alkaline phosphatase as described herein. In embodiments of the methods, at least one of a mutant heat tolerant alkaline phosphatase of SEQ ID NO: 1, 2, 3 or 18-73, or a heat tolerant variant thereof are administered. In embodiments of the methods, a mutant heat tolerant alkaline phosphatase of SEQ ID NO: 2, or a heat tolerant variant thereof are administered. In embodiments of the methods, a mutant heat tolerant alkaline phosphatase of SEQ ID NO: 1, or a heat tolerant variant thereof are administered. In embodiments of the methods, a mutant heat tolerant alkaline phosphatase of SEQ ID NO: 1 is administered.

In one embodiment, the present disclosure provides a method for reducing bacterial lipopolysaccharides (LPS) in the intestine in a subject. In one embodiment, the present disclosure provides a method of reducing inflammation particularly bacterial-mediated inflammation in a subject. In one embodiment, the present disclosure provides a method of reducing production of pro-inflammatory cytokines, particularly in the intestine in a subject. In one embodiment, the present disclosure provides a method of improving intestinal permeability markers in a subject. In one embodiment, the present disclosure provides a method of improving performance parameters in a subject. In one embodiment, the present disclosure provides a method of improving nutrient utilization or providing more efficient nutrient utilization in a subject. In one embodiment, the present disclosure provides a method of alleviating or modulating increased intestinal permeability or leaky gut in a subject. In one embodiment, the present disclosure provides a method of reducing intestinal permeability in a subject. The method(s) includes administering an effective amount of at least one of a mutant heat tolerant alkaline phosphatase as described herein. In embodiments of the methods, at least one of a mutant heat tolerant alkaline phosphatase of SEQ ID NO: 1, 2, 3 or 18-73, or a heat tolerant variant thereof are administered.

In an embodiment, the method(s) comprises administering an effective amount of a composition comprising at least one of a mutant heat tolerant alkaline phosphatase as described herein, and optionally, an anti-inflammatory agent, molecule or cytokine, to a subject. In embodiments of the methods, at least one of a mutant heat tolerant alkaline phosphatase of SEQ ID NO: 1, 2, 3 or 18-73, or a heat tolerant variant thereof are administered and optionally, an anti-inflammatory agent, molecule or cytokine are administered, to a subject. In embodiments of the methods, at least one of a mutant heat tolerant alkaline phosphatase of SEQ ID NO: 2, or a heat tolerant variant thereof are administered and optionally, an anti-inflammatory agent, molecule or cytokine are administered, to a subject. In embodiments of the methods, at least one of a mutant heat tolerant alkaline phosphatase of SEQ ID NO: 1, or a heat tolerant variant thereof are administered and optionally, an anti-inflammatory agent, molecule or cytokine are administered, to a subject. In embodiments of the methods, at least one of a mutant heat tolerant alkaline phosphatase of SEQ ID NO: 1 is administered and optionally, an anti-inflammatory agent, molecule or cytokine are administered, to a subject.

In an embodiment, the method(s) comprises administering an effective amount of a composition comprising at least one of a mutant heat tolerant alkaline phosphatase as described herein, and optionally, an anti-bacterial and/or an anti-infective agent, to a subject. In embodiments of the methods, at least one of a mutant heat tolerant alkaline phosphatase of SEQ ID NO: 1, 2, 3 or 18-73, or a heat tolerant variant thereof are administered and optionally, an anti-bacterial and/or an anti-infective agent, to a subject.

In one embodiment, the present disclosure provides a method of reducing inflammation, including intestinal-derived or intestinal-associated inflammation, including inflammation associated with bacterial LPS, or intestinal disease, or intestinal bacterial infection, in a subject. The method includes administering an effective amount of a composition having at least one of a mutant heat tolerant alkaline phosphatase of SEQ ID NO: 1, 2, 3 or 18-73, or a heat tolerant variant thereof. The method includes administering an effective amount of a composition having at least one of a mutant heat tolerant alkaline phosphatase of SEQ ID NO: 2, or a heat tolerant variant thereof. The method includes administering an effective amount of a composition having at least one of a mutant heat tolerant alkaline phosphatase of SEQ ID NO: 1, or a heat tolerant variant thereof. In an embodiment, the method comprises administering one or more enzyme of SEQ ID NO: 1, 2 or 3 or a composition comprising at least one of a mutant heat tolerant alkaline phosphatase of SEQ ID NO: 1, 2 or 3, and optionally, an anti-inflammatory agent, molecule or cytokine, to a subject. In an embodiment, the method comprises administering one or more enzyme of SEQ ID NO: 1, 2, 3 or 18-73 or a composition comprising at least one of a mutant heat tolerant alkaline phosphatase of SEQ ID NO: 1, 2, 3, or 18-73, and optionally, an anti-inflammatory agent, molecule or cytokine, to a subject. In an embodiment, the method comprises administering one or more enzyme of SEQ ID NO: 2 or a composition comprising a mutant heat tolerant alkaline phosphatase of SEQ ID NO: 2, and optionally, an anti-inflammatory agent, molecule or cytokine, to a subject. In an embodiment, the method comprises administering one or more enzyme of SEQ ID NO: 1 or a composition comprising a mutant heat tolerant alkaline phosphatase of SEQ ID NO: 1, and optionally, an anti-inflammatory agent, molecule or cytokine, to a subject.

In an embodiment of the method(s), at least one of a mutant heat tolerant alkaline phosphatase of SEQ ID NO: 1, 2, 3 or 18-73, or a heat tolerant variant thereof, is administered. In embodiments, an anti-inflammatory agent, molecule or cytokine or an immunomodulatory agent is administered in combination with the at least one of a mutant heat tolerant alkaline phosphatase of SEQ ID NO: 1, 2, 3 or 18-73, or a heat tolerant variant thereof. The at least one of a mutant heat tolerant alkaline phosphatase of SEQ ID NO: 1, 2, 3 or 18-73, or a heat tolerant variant thereof may be administered prior to one or more anti-inflammatory agent, molecule or cytokine or immunomodulator; may be administered prior to and in conjunction one or more anti-inflammatory agent, molecule or cytokine or immunomodulator; may be administered prior to, in conjunction with, and following one or more anti-inflammatory agent, molecule or cytokine or immunomodulator; or may be administered in combination with or shortly following one or more anti-inflammatory agent, molecule or cytokine or immunomodulator. The one or more anti-inflammatory agent, molecule or cytokine or immunomodulator may be administered as a single dose or multiple doses. The at least one of a mutant heat tolerant alkaline phosphatase of SEQ ID NO: 1, 2, 3 or 18-73, or a heat tolerant variant thereof may be administered prior to and/or between and/or in combination with a dose of one or more anti-inflammatory agent, molecule or cytokine or immunomodulator or multiple doses of one or more anti-inflammatory agent, molecule or cytokine or immunomodulator.

In another embodiment, the invention includes a composition, said composition comprising the mutant alkaline phosphatase enzyme polypeptide as provided herein. In an embodiment, the composition further comprises an additive or diluent or carrier, such as a pharmaceutically acceptable carrier or a digestible carrier. In an embodiment, the invention includes an anti-inflammatory or anti-bacterial composition, said composition comprising one or more mutant alkaline phosphatase enzyme polypeptide as provided herein and an additive or diluent or carrier, such as a pharmaceutically acceptable carrier or a digestible carrier. In an embodiment, the invention includes a feed composition or feed additive, said composition comprising a mutant alkaline phosphatase enzyme polypeptide as provided herein.

In one embodiment, the present disclosure provides a method of reducing inflammation, including bacterial-mediated inflammation, in the gut or intestine in a subject. In one embodiment, the present disclosure provides a method of reducing production of pro-inflammatory cytokines in the gut or intestine in a subject. In one embodiment, the present disclosure provides a method of improving nutrient utilization in a subject. In one embodiment, the present disclosure provides a method of increasing alkaline phosphatase (AP) production and activity in a subject. In one embodiment, the present disclosure provides a method of increasing AP production and activity in the duodenum, jejunum and ileum of an animal. In one embodiment, the present disclosure provides a method of reducing TNFα expression, particularly in the presence of LPS, in a subject, including in the intestine of a subject. In one embodiment, the present disclosure provides a method of reducing IL-6 expression, particularly in the presence of LPS, in a subject, including in the intestine of a subject. In one embodiment, the present disclosure provides a method of promoting inorganic phosphate release from LPS, in a subject, including in the intestine of a subject. In one embodiment, the present disclosure provides a method of improving growth and performance parameters in a subject, particularly in a livestock animal. Any such method includes administering an effective amount of a composition comprising at least one of a mutant heat tolerant alkaline phosphatase of SEQ ID NO: 1, 2, 3 or 18-73, or a heat tolerant variant thereof to the subject or the animal.

The method includes further administering one or more anti-inflammatory agent, molecule or cytokine or immunomodulatory to a subject. The method includes further administering, including in combination in the composition, one or more anti-inflammatory agent, molecule or cytokine or immunomodulatory to a subject.

In one embodiment, the present disclosure provides a method of reducing intestinal inflammation associated with gastrointestinal disease including or such as inflammatory bowel disease (IBD), Crohn's disease, or celiac disease in a subject. The method includes administering an effective amount of a composition comprising at least one of a mutant heat tolerant alkaline phosphatase of SEQ ID NO: 1, 2, 3 or 18-73, or a heat tolerant variant thereof to the subject or the animal. The method includes further administering one or more anti-inflammatory agent, molecule or cytokine or immunomodulatory to a subject. The method includes further administering, including in combination in the composition, one or more anti-inflammatory agent, molecule or cytokine or immunomodulatory to a subject.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1 depicts a comparison of the AP1 alkaline phosphatase (designated AP) versus calf intestinal alkaline phosphatase (designated IAP) in reducing lipopolysaccharide (LPS)-induced inflammation as evaluated by expression of the proinflammatory cytokine TNFα (TNFα mRNA expression). The study was conducted using primary alveolar porcine macrophages (RAW264.7 cells)

FIG. 2 depicts thermostability of alkaline phosphatase mutant designated 2051 versus the parent alkaline phosphatase AP1 (designated 1941). Residual activity on a scale of 0% to 100% (100% being starting baseline activity) after 20 minutes of incubation at the indicated temperature is presented.

FIG. 3 provides amino acid sequence comparison of the parent AP1 alkaline phosphatase enzyme versus the 2051 mutant alkaline phosphatase. The sequences include a leader of several amino acids particularly ASRA (SEQ ID NO:10) amino acid N-terminal sequence. Only amino acid changes or mutations are indicated and are shown in bold and underlined. Identical amino acids are shown with a dash (-). The AP1 enzyme with ASRA leader is provided as SEQ ID NO:11. The 2051 mutant enzyme with ASRA leader sequence is provided as SEQ ID NO:12.

FIG. 4 depicts kill curve in percent recovery of alkaline phosphatase mutant designated 2051 versus the heat tolerant mutant alkaline phosphatase APHT. Residual activity on a scale of 0% to 100% (100% being starting baseline activity of APHT) after 15 minutes of exposure at the indicated temperature is presented.

FIGS. 5A and 5B provides amino acid sequence comparisons of the parent AP1 alkaline phosphatase enzyme versus the APHT mutant alkaline phosphatase. The sequences include a leader of several amino acids particularly ASGFYVSGT amino acid N-terminal sequence. Only amino acid changes or mutations are indicated. Identical amino acids are shown with a dot (●). In FIG. 5A, the AP1 with a serine mutation and the APHT are depicted in sequence with a leader sequence ASGFYVSGT (SEQ ID NO:13), the leader shown in bold. In FIG. 5B, the AP1 with a serine mutation and without any leader sequence or leader peptide amino acids is aligned and compared with the APHT without any leader sequence or leader peptide amino acids. The 14 amino acid changes in APHT are evident in A and B and are underlined and in bold. The AP1 with leader sequence ASGFYVSGT is provided in SEQ ID NO:14). The APHT polypeptide with leader sequence ASGFYVSGT is provided in SEQ ID NO:15). The APHT amino acid sequence (excluding and without any leader sequence and starting with amino acids ESSN) (SEQ ID NO:1) is 97% identical to the sequence of AP1 (excluding and without any leader sequence and starting with amino acids ESSN) (SEQ ID NO:4), as shown in B.

FIG. 6 provides a comparison of the pH activity profile of AP1 alkaline phosphatase (denoted as Native AP) versus APHT (denoted as HT AP). Relative activity is graphed versus pH.

FIG. 7 presents effect of pH on (A)Vmax (IU/mg) and (B) KM (μM) activity of AP1 (designated “Native”) and APHT (designated “HT”). Assays were performed at 40° C. with p-NPP as substrate in the concentration range 0-5 mM. The enzyme concentration was 2.5 μg/mL for the pH 8 and 0.4 mg/mL for pH 9-13).

FIG. 8 presents effect of pH on (A)Vmax (IU/mg) and (B) KM (μM) activity of AP1 (designated “Native”) and APHT (designated “HT”). Assays were performed at 50° C. with p-NPP as substrate in the concentration range 0-5 mM. The enzyme concentration was 2.5 μg/mL for the pH 8 and 0.4 mg/mL for pH 9-13).

FIG. 9 depicts (A)Vmax (IU/mg) and (B) KM (μM) activity of AP1 (designated “Native”) and APHT (designated “HT”) on UDP, ATP and dAMP. Assays were performed at 40° C. with p substrate concentration 0-5 mM in Tris buffer pH 8. The enzyme concentration was 2 μg/mL.

FIG. 10 depicts (A)Vmax (IU/mg) and (B) KM (μM) activity of AP1 (designated “Native”) and APHT (designated “HT”) with UDP, ATP and dAMP. Assays were performed at 50° C. with p substrate concentration 0-5 mM in DEA buffer pH 10. The enzyme concentration was 2 μg/mL.

FIG. 11 depicts Pi (μM) produced after 5 min of reaction of 0.4 mg/mL AP1 (designated Native) and APHT (designated HT) with different LPS and substrates (see Table 9), assayed in (A) at 40° C. and in (B) at 50° C. The LPS and substrate concentration was 0.1-1 mg/mL (see Table 3). Background signal from controls for each substrate containing no AP and treated as reaction was subtracted from all measurement prior to activity calculations.

FIG. 12 depicts inorganic phosphorous release (μM) with various LPS sources, particularly L3137, L2143, L2262, L6511, L6011 and L9413.

FIG. 13 depicts TNF-α and IL-6 gene expression and TNF-α plasma concentration. (A) provides TNF-α relative gene expression without LPS and then with Salmonella typhimurium LPS alone, or with addition of APHT (AP) or commercially available intestinal alkaline phosphatase (Sigma-IAP). (B) provides IL-6 relative gene expression without LPS and then with Salmonella typhimurium LPS alone, or with addition of APHT (AP) or commercially available intestinal alkaline phosphatase (Sigma-IAP). (C) shows plasma concentration of TNF-α in weaned males pigs injected i.p. with 10 mg/kg BW of lipopolysaccharide from Salmonella enterica serotype Typhimurium (Sigma L2262).

FIG. 14 depicts duodenum and jejunum alkaline phosphatase (ALP) present in swine fed a control diet or supplemented with AP in the muosa and the digesta.

FIG. 15 depicts alkaline phosphatase (ALP) activity (U/ml) in the jejunum of broilers fed a control diet or supplemented with AP.

FIG. 16 provides evaluation of (A) intestinal alkaline phosphatase (IAP) relative gene expression and (B) ZO-1 relative gene expression in the duodenum, jejunum and ileum of swine fed a control diet or fed diet supplemented with APHT.

### DETAILED DESCRIPTION OF THE INVENTION

In accordance with the present invention there may be employed conventional molecular biology, microbiology, and recombinant DNA techniques within the skill of the art. Such techniques are explained fully in the literature.

The disclosure provides methods for reducing bacterial lipopolysaccharides (LPS) in the intestine, reducing inflammation particularly bacterial-mediated inflammation, reducing production of pro-inflammatory cytokines, particularly in the intestine, improving intestinal permeability markers, improving performance parameters, and improving or resulting in more efficient nutrient utilization in an animal, all and any of which include administration of an effective amount of at least one of a mutant heat tolerant alkaline phosphatase as described herein. The invention provides methods for reducing intestinal permeability, alleviating alcohol induced or disease related leaky gut syndrome, alleviating the intestinal and systemic effects of chronic alcohol consumption, including intestinal dysbiosis, and reducing inflammation, including intestinal-derived or intestinal-associated inflammation, all and any of which include administration of an effective amount of at least one of a mutant heat tolerant alkaline phosphatase as described herein. In embodiments of the methods, at least one of a mutant heat tolerant alkaline phosphatase of SEQ ID NO: 1, 2, 3 or 18-73, or a heat tolerant variant thereof are administered.

The disclosure provides a composition comprising one or more mutant heat tolerant alkaline phosphatase, wherein said composition increases animal health when an effective amount is administered to an animal, as compared to an animal not administered the composition. In one embodiment, the invention provides a composition having at least one of a mutant heat tolerant alkaline phosphatase of SEQ ID NO: 1, 2, 3 or 18-73, or a heat tolerant variant thereof, wherein said composition increases animal health when an effective amount is administered to an animal, as compared to an animal not administered the composition.

The present invention provides methods for reducing the environmental impact of animal waste. In particular, the invention provides methods comprising administering to an animal an enzyme that is effective to reduce the amount of a detrimental compound present in or released from animal waste, and compositions suitable for use in such methods. Also provided is a method for increasing phosphorus digestion in an animal.

Animal waste may contain or release one or more compounds that have a detrimental effect, such as a detrimental effect on the animal, on other animals, on humans, or on the environment. One such compound is ammonia (NH₃). Another such compound is phosphorous (P).

Atmospheric ammonia can have adverse effects on the environment, as well as on animal production performance, health, and welfare. Ammonia generation and emission in, for example, poultry housing, mostly result from the microbiological decomposition of poultry waste. Ammonia levels as low as 50 ppm can be detrimental to poultry, and such low levels may go unnoticed. Exposure to ammonia at 50 ppm can contribute to 5-10% of birds being runts, and can be associated with a loss of 0.5 pounds of meat per bird and/or a loss of 8 points of feed conversion.

There is a need, therefore, for methods of reducing the amount of a detrimental compound present in or released from animal waste, such as for reducing the ammonia and/or the phosphorous content of animal waste.

In accordance with some embodiments, there are provided methods for reducing the environmental impact of animal waste, comprising administering to an animal an effective amount of an enzyme that reduces the amount of a detrimental compound present in or released from animal waste. In accordance with some embodiments, there are provided methods for reducing the amount of ammonia in animal waste, comprising administering to an animal an effective amount of an enzyme that reduces the amount of ammonia present in or released from animal waste. In accordance with some embodiments, there are provided methods for reducing the amount of phosphorous in animal waste, comprising administering to an animal an effective amount of an enzyme that reduces the amount of phosphorous present in or released from animal waste. Also provided is a method for increasing phosphorus digestion in an animal, comprising administering an effective amount of alkaline phosphatase to the animal.

The present disclosure also provides methods of increasing animal health, wherein the method includes administering an effective amount of the composition to an animal. Methods are provided for reducing bacterial lipopolysaccharides (LPS) in the intestine, reducing inflammation particularly bacterial-mediated inflammation, reducing production of pro-inflammatory cytokines, particularly in the intestine, improving intestinal permeability markers, improving performance parameters, and improving or resulting in more efficient nutrient utilization in an animal, all and any of which include administration of an effective amount of at least one of a mutant heat tolerant alkaline phosphatase as described herein. Methods are provided for reducing intestinal permeability, alleviating alcohol induced or disease related leaky gut syndrome, alleviating the intestinal and systemic effects of chronic alcohol consumption, including intestinal dysbiosis, and reducing inflammation, including intestinal-derived or intestinal-associated inflammation, all and any of which include administration of an effective amount of at least one of a mutant heat tolerant alkaline phosphatase as described herein.

Methods for reducing or alleviating or preventing LPS-mediated inflammation, particularly bacterial LPS-mediated inflammation, and compositions for use in reducing or alleviating or preventing LPS-mediated inflammation, particularly bacterial LPS-mediated inflammation are provided herein.

In accordance with any of these methods, the alkaline phosphatase may be the mutant heat tolerant alkaline phosphatase enzyme provided herein. In accordance with any of these methods, the enzyme may be the mutant heat tolerant AP enzyme as set out in SEQ ID NO: 1, 2 or 3 or heat tolerant variants thereof. In accordance with any of these methods, the enzyme may be the mutant heat tolerant AP enzyme as set out in SEQ ID NO: 1, 2, 3, or 18-73. The mutant heat tolerant AP enzyme may further comprise or include an N terminal leader or additional N terminal amino acid sequences, particularly wherein the N terminal leader or additional N terminal amino acid sequences do not alter or significantly reduce its activity.

In accordance with any of these methods, the enzyme may be administered orally.

In accordance with any of these methods, the enzyme may be alkaline phosphatase.

In accordance with any of these methods, the animal may be human or a non-human animal or a bird. In accordance with any of these methods, the animal may be a livestock animal. In accordance with any of these methods, the animal may be a poultry or swine animal. In accordance with any of these methods, the animal may be a poultry or swine or bovine (cattle) animal.

In accordance with any of these methods, the enzyme may be administered during one or more of the starter phase, the grower phase, and/or the finisher phase. In accordance with any of these methods, the enzyme may be administered during post-weaning, or after weaning phase. In accordance with any of these methods, the enzyme may be administered to a very young animal shortly after birth or shortly after weaning.

In accordance with any of these methods, the enzyme may be formulated in animal feed or may be formulated in a digestible or food format. In accordance with any of these methods, the enzyme may be formulated in animal feed, such as a starter feed, a grower feed, or a finisher feed.

In accordance with any of these methods, the enzyme may be formulated in a feed additive or in a probiotic composition. In accordance with any of these methods, the enzyme may be formulated in an animal feed additive.

In accordance with some embodiments, there are provided compositions suitable for oral administration to an animal, comprising an effective amount of an enzyme that reduces the amount of a detrimental compound present in or released from animal waste. In accordance with some embodiments, there are provided compositions suitable for oral administration to an animal, comprising an effective amount of an enzyme that reduces the amount of ammonia present in or released from animal waste. In accordance with some embodiments, there are provided compositions suitable for oral administration to an animal, comprising an effective amount of an enzyme that reduces the amount of phosphorous present in or released from animal waste. In accordance with some embodiments, there are provided compositions suitable for oral administration to an animal, comprising an effective amount of an enzyme that reduces bacterial lipopolysaccharides (LPS) in the intestine, reduces inflammation particularly bacterial-mediated inflammation, reduces production of pro-inflammatory cytokines, particularly in the intestine, improves intestinal permeability markers, improves performance parameters, and/or improves or results in more efficient nutrient utilization in an animal. In accordance with some embodiments, there are provided compositions suitable for oral administration to an animal, comprising an effective amount of an enzyme that reduces intestinal permeability, alleviates alcohol induced or disease related leaky gut syndrome, alleviates the intestinal and systemic effects of chronic alcohol consumption, including intestinal dysbiosis, and/or reduces inflammation, including intestinal-derived or intestinal-associated inflammation. All and any of the compositions comprise an effective amount of at least one of a mutant heat tolerant alkaline phosphatase as described herein.

In accordance with any of these compositions, the composition may comprise an orally acceptable carrier for the enzyme.

In accordance with any of these compositions, the enzyme may be alkaline phosphatase. In accordance with any of these compositions, the enzyme may be the mutant heat tolerant alkaline phosphatase enzyme provided herein. In accordance with any of these compositions, the enzyme may be the mutant heat tolerant AP enzyme as set out in SEQ ID NO: 1, 2 or 3 or heat tolerant variants thereof. In accordance with these compositions, the enzyme may be the mutant heat tolerant AP enzyme as set out in SEQ ID NO: 1, 2, 3, 7, 8, 12, 15, or 18-73. In accordance with these compositions, the enzyme may be the mutant heat tolerant AP enzyme as set out in SEQ ID NO: 1, 2, 3, or 18-73, or heat tolerant variants thereof. In accordance with these compositions, the enzyme may be the mutant heat tolerant AP enzyme as set out in SEQ ID NO: 1, 2, 3, or 18-73. The mutant heat tolerant AP enzyme may further comprise or include an N terminal leader or additional N terminal amino acid sequences, particularly wherein the N terminal leader or additional N terminal amino acid sequences do not alter or significantly reduce its activity.

Any of these compositions may be suitable for administration to human or non-human animals, including poultry or swine. Any of these compositions may be suitable for administration to poultry or swine or cattle. Any of these compositions may be suitable for administration to an animal. Any of these compositions may be suitable for administration to a human.

Any of these compositions may be an animal feed, such as a starter diet, a grower diet, or a finisher diet, or may be an animal feed additive.

As used in the following discussion, the terms “a” or “an” should be understood to encompass one or more, unless otherwise specified.

As used herein, the term “animal” refers to any animal, including birds, humans and other non-human mammals or animals. Specific examples of birds include poultry such as chickens or turkey. The term animal includes companion animals such as dogs and cats, livestock, such as cows and other ruminants, buffalo, horses, swine (e.g., pigs or hogs), sheep, fowl or poultry (e.g., chicken, ducks, turkeys, and geese) and aquaculture animals (e.g., fish and shrimp and eels). A young animal is an animal which falls into the starter (or pre-starter) or grower category. Preferably, the young animal falls into the starter (or pre-starter) category. For swine, an animal less than 25 kilograms is also considered a young animal.

The composition disclosed herein and above increases animal health by providing positive health benefits when administered to an animal, as compared to an animal that has not been administered the composition.

Positive health benefits include decreasing feed conversion ratio, increasing weight, increasing lean body mass, decreasing pathogen-associated lesion formation in the gastrointestinal tract, decreasing colonization of pathogens, reducing inflammation, and decreasing mortality rate. Positive health benefits described, demonstrated and provided herein include reducing bacterial lipopolysaccharides (LPS) in the intestine, reducing inflammation particularly bacterial-mediated inflammation, reducing production of pro-inflammatory cytokines, particularly in the intestine, improving intestinal permeability markers, improving performance parameters, and improving or resulting in more efficient nutrient utilization in an animal. Positive health benefits described, demonstrated and provided herein include reducing intestinal permeability, addressing leaky gut syndrome or symptoms thereof, and for reducing or blocking inflammation, including inflammation and inflammatory responses or symptoms, including those associated with altered intestinal permeability, leaky gut, or leaky gut syndrome.

Inflammation, particularly intestinal inflammation, including bacterial-mediated intestinal inflammation, such as via or resulting from bacterial LPS, is important and relevant to various clinical conditions and symptoms and problems, including overall animal performance and growth. Altered or compromised intestinal permeability and intestinal dysbiosis are important and relevant to various clinical conditions and symptoms and problems. The most direct causes of altered or increased intestinal permeability include: chronic inflammatory states, such as IBD and celiac disease; other diseases that cause intestinal injury, such as HIV/AIDS; chemotherapy and radiation therapies that degrade the intestinal mucosa; chronic overuse of alcohol or NSAIDs, such as aspirin and ibuprofen; food allergies that cause an immune response to certain foods. Intestinal permeability is a recognized feature of several inflammatory and autoimmune diseases affecting the digestive system, including inflammatory bowel disease, Crohn's disease and celiac disease.

There are both pro-inflammatory and anti-inflammatory molecules or cytokines. The pro-inflamatory cytokines are secreted from Th1 cells. CD4⁺ cells, macrophages, and dendritic cells, They are characterized by production of several Interleukins (TL), IL-1, L-2, IL-12, IL-1 IL-18, IFN-γ, and TNF-α. The key pro-inflammatory cytokines are IL-1, IL-6, and TNF-α. Pro-inflammatory chemokines are produced by cells primarily to recruit leukocytes to the sites of infection or injury. They are crucial for coordinating cell mediated immune response and play a critical role in modulating the immune system. Pro-inflammatory cytokines generally regulate growth, cell activation, differentiation, and homing of the immune cells to the sites of infection with the aim to control and eradicate intracellular pathogens. TL-1 is subdivided in IL-1α and IL-1β IL-1β is potent pro-inflammatory cytokine, induced mainly by lymphocytes, macrophages, and monocytes in response to microbial molecules, The anti-inflammatory cytokines are a series of immunoregulatory molecules that control the proinflammatory cytokine response. Anti-inflammatory cytokines include IL-10, which inhibits cytokine production and mononuclear cell function, IL-12, which activates NK cells, IL-22, which stimulates cell survival and proliferation, and TGF-β, which Inhibits T and B cell proliferation. Anti-inflammatory interleukins include interleukin (IL)-1 receptor antagonist, IL-4, IL-6, IL-10, IL-13, IL-19 and IL-35.

The studies set out and provided herein demonstrate that administration of the compositions and mutant heat tolerant alkaline phosphatase provided herein results in reduced intestinal inflammation, bacterial LPS, and inflammatory cytokines. The studies set out and provided herein further demonstrate that administration of the compositions and heat tolerant mutant AP described results in reduced levels of pro-inflammatory cytokines, including IL-6 and TNF-α. The studies set out and provided herein further demonstrate that administration of the compositions and heat tolerant mutant AP described results in inactivation and dephosphorylation of LPS, including particularly bacterial LPS in an animal.

In some embodiments, the compositions disclosed herein reduce pro-inflammatory molecules or cytokines by at least 10%, at least 20%, at least 25%, at least 50%, at least 60%, at least 80%. In some embodiments, the compositions disclosed herein reduce pro-inflammatory molecules or cytokines by at least 1 fold, 2 fold, 3 fold, 4 fold. In some embodiments, the compositions disclosed herein reduce pro-inflammatory molecules or cytokines IL-6, TNF-α, IFN-γ, and/or IL-1β by at least 10%, at least 20%, at least 25%, at least 50%, at least 60%, at least 80%. In some embodiments, the compositions disclosed herein reduce pro-inflammatory molecules or cytokines IL-6, TNF-α, IFN-γ, and/or IL-1 by at least 1 fold, 2 fold, 3 fold, 4 fold, 6 fold, 8 fold, 10 fold.

The composition of one or more mutant heat tolerant alkaline phosphatase may be combined with one or more other or anti-inflammatory agent, molecule or cytokine or immune modulator. Immune modulators may include cytokines, hormones, antibodies which modulate, including to particularly reduce or alleviate the immune response or inflammatory response. The composition of one or more mutant heat tolerant alkaline phosphatase may be combined with one or more anti-inflammatory drug or immune suppressants/immune modulator, including the one or more drug or modifier described herein. The composition of one or more mutant heat tolerant alkaline phosphatase may be combined with one or more anti-inflammatory, nonsteroidal anti-inflammatory drug (NSAID), steroid, biologic, antibiotic, or anti-diarrheal agent, including as described above. The composition of one or more mutant heat tolerant alkaline phosphatase may be combined with an anti-inflammatory cytokine such as IL-10, IL-12 or IL-22. The composition of one or more mutant heat tolerant alkaline phosphatase may be combined with an IL-1 inhibitor, such as an IL-1 receptor antagonist.

Described herein are methods comprising administering to an animal an enzyme that is effective to reduce the amount of a detrimental compound present in or released from animal waste, such as ammonia (NH₃) or phosphorous (P), and compositions suitable for use in such methods. The methods offer a number of advantages in the context of animal production, including poultry and swine production. For example, the methods may offer advantages such as reduced phosphate input into an animal production system, decreased ammonia in animal manure, reduced ventilation air requirements to dilute indoor ammonia concentration in animal housing (and associated energy savings), and reduced need to further treat exhaust air.

In accordance with the invention are compositions and methods for improving animal health, including animal growth and/or animal performance. In accordance with the invention are compositions and methods for improving animal health, including reducing or controlling bacteria-mediated inflammation, particularly in the gut or intestine of an animal.

While not wanting to be bound by any theory, the results reported below indicate that the methods described herein may help animals (such as young broilers and piglets) utilize and digest the phosphorus that is present in their diets, which in turn may lead to better growth rate and less nutrient loss through excretion. Additionally or alternatively, the methods described herein may decrease NH₃ emission because the enzyme treatments may increase the metabolism and growth of favorable bacterial populations in the intestine, such that more of the excess nitrogen in the diet remains in the manure as bacterial protein instead of uric acid, which is typically degraded and emitted as NH₃. Moreover, both the lower pH and lower nitrogen content in manure of treated animals may deter and prevent the formation of gaseous NH₃ in the manure and reduce the NH₃ emission. The relationship between pH and degradation of uric acid (the major nitrogen source in poultry manure) has been reported such that a sharp increase in pH may be associated with a decrease in the uric acid content of poultry manure. Elliot & Collins, 1982, Transactions of ASAE 25: 413-24, indicated that high pH in the stored manure would result in the majority of nitrogen loss as NH₃. Additionally, reducing the phosphorus content of animal waste may impact other properties of the manure, such as the bacterial flora. Additionally or alternatively, the methods described herein reduce inflammation, particularly intestinal inflammation, including particularly bacterial-mediated intestinal inflammation. Additionally or alternatively, the methods described herein reduce or inactivate lipopolysaccharide (LPS), particularly bacterial LPS.

In specific embodiments, the enzyme is mutant alkaline phosphatase, particularly mutant heat tolerant alkaline phosphatase, as provided herein, which is mutated from bacterial native AP enzyme, corresponding in aspects to alkaline phosphatase (AP) (EC 3.1.3.1). In specific embodiments, the enzyme is mutant heat tolerant AP enzyme as set out in SEQ ID NO: 1, 2, 3, or 18-73, or heat tolerant variants thereof. Alkaline phosphatases occur in prokaryotic and eukaryotic organisms, including mammals (including humans). For example, alkaline phosphatase is naturally present in breast milk and intestines, and plays a key role in digestion and digestion regulation. Alkaline phosphatase has been studied for use in therapeutic contexts (e.g., the treatment of cancer, diabetes and weight loss). Comparison of the primary structures of various alkaline phosphatases showed a high degree of homology (25-30% homology between E. coli and mammalian). Millan, 1988 Anticancer Res. 8, 995-1004; Harris, 1989 Clin. Chim. Acta 186, 133-150. The alkaline phosphatase family includes the tissue-specific APs (placental AP (PLAP), germ cell AP (GCAP) and intestinal AP (IAP>> and the non-tissue specific APs (TnAP) which are primarily located in the liver, kidney and bones. U.S. Pat. No. 6,884,602 reports the expression of alkaline phosphatase in yeast. Hundreds of microbial alkaline phosphatases have been described. See, e.g., BRENDA: The Comprehensive Enzyme Information System, on the web at brenda-enzymes.orglphp/resultflat.php4?ecno=3.1.3.1. Moreover, organisms can be engineered to produce enzymes with desired properties, such as increased activity. See, e.g., Du et al. J. Mol. Biol. 316: 941-53 (2002); Dealwis et al., Biochem. 34: 13967-73 (1995); Koutsioulis et al., Protein Eng'g Design & Selection 21: 319-27 (2008).

As used herein, pathogen includes Salmonella, Clostridium, Campylobacter, Staphylococcus, Streptococcus, and E. coli bacterium. Further examples of pathogens include Salmonella typhimurium, Salmonella infantis, Salmonella Hadar, Salmonella enteritidis, Salmonella Newport, Salmonella Kentucky, Clostridium perfringens, Staphylococcus aureus, Streptoccus uberis, Streptococcus suis, Escherichia coli, Campylobacter jejuni, and Fusobacterium necrophorum.

The invention also provides a mutant and heat tolerant alkaline phosphatase of SEQ ID NO:1, SEQ ID NO:2 or SEQ ID NO:3, or an alkaline phosphatase having at least 98% sequence identity with SEQ ID NO:1, SEQ ID NO: 2 or SEQ ID NO:3. The invention provides a mutant and heat tolerant alkaline phosphatase of SEQ ID NO: 1, 2, 3, or 18-73, or an alkaline phosphatase having at least 98% sequence identity with SEQ ID NO: 1, 2, 3, or 18-73. The mutant heat tolerant AP enzyme may further comprise or include an N terminal leader or additional N terminal amino acid sequences, particularly wherein the N terminal leader or additional N terminal amino acid sequences do not alter or significantly reduce its activity.

The invention also provides alkaline phosphatases having at least 95, 96, 97, 98, or 99% sequence identity with SEQ ID NO:1, SEQ ID NO:2 or SEQ ID NO:3. The invention also provides alkaline phosphatases having at least 95, 96, 97, 98, or 99% sequence identity with SEQ ID NO: 1, 2, 3, or 18-73.

In an embodiment, a mutant heat tolerant alkaline phosphatase enzyme is provided having or comprising the amino acid sequence set out in SEQ ID NO: 1 or having at least 95%, 96%, 97%, 98% or 99% identity with SEQ ID NO:1. In an embodiment, the mutant heat tolerant alkaline phosphatase enzyme does not have or comprise SEQ ID NO:4.

In an embodiment, a mutant heat tolerant alkaline phosphatase enzyme is provided having or comprising the amino acid sequence set out in SEQ ID NO: 2 or having at least 95%, 96%, 97%, 98% or 99% identity with SEQ ID NO:2. In an embodiment, the mutant heat tolerant alkaline phosphatase enzyme does not have or comprise SEQ ID NO:4.

In an embodiment, a mutant heat tolerant alkaline phosphatase enzyme is provided having or comprising the amino acid sequence set out in SEQ ID NO: 3 or having at least 95%, 96%, 97%, 98% or 99% identity with SEQ ID NO:3. In an embodiment, the mutant heat tolerant alkaline phosphatase enzyme does not have or comprise SEQ ID NO:4.

In an embodiment, a mutant heat tolerant alkaline phosphatase enzyme is provided having or comprising the amino acid sequence set out in any of SEQ ID NOs: 18-73 or having at least 95%, 96%, 97%, 98% or 99% identity with any of SEQ ID NOs: 18-73. In an embodiment, the mutant heat tolerant alkaline phosphatase enzyme does not have or comprise SEQ ID NO:4.

The invention also provides compositions containing at least one of the above mutant heat tolerant alkaline phosphatases, as well as methods of using such an alkaline phosphatase for reducing the amount of one or more detrimental compounds present in or released from animal waste, increasing animal feed conversion rate, increasing animal feed efficiency, and/or increasing animal growth rate.

Sequence identity refers to a sequence that has a specified percentage of amino acid residues that are the same (i.e., share at least 90% identity, for example), when compared and aligned for maximum correspondence over a comparison window, or designated region as measured using a sequence comparison algorithms or by manual alignment and visual inspection.

In specific embodiments, the methods comprise administering to an animal an amount of an enzyme, such as alkaline phosphatase, effective to reduce the amount of ammonia (NH₃) or phosphorous present in or released from the animal's waste. The amount may vary depending on the animal, the animal's diet, and other factors, and can readily be determined by those skilled in the art using methods known in the art and illustrated in the examples. For example, the amount of ammonia (NH₃) and/or phosphorous present in or released from animal waste when given animals are grown under given conditions can be measured and compared to that present in or released from the animal waste of animals grown under comparable conditions, but also administered an amount of the enzyme, such as alkaline phosphatase. (In this regard, it may be advantageous to compare treated and control animals of the same age, as manure properties may change with age, as discussed in the examples below). A decrease in manure ammonia (NH₃) and/or phosphorous content or release associated with administration of the enzyme indicates that an effective amount of enzyme was administered.

The compositions and/or enzymes may be administered orally, parentally, nasally, or mucosally. Parental administration includes subcutaneous, intramuscular and intravenous administrator. The enzyme typically is administered orally. However, the invention also encompasses embodiments where the enzyme is administered by other routes to the intestines or digestive tract, in accordance with known practices, such as via suppositories. The enzyme may be administered in food or as a feed additive.

In some aspects, administration includes feeding the poultry, or spraying onto the poultry. In other aspects, administration includes on ovo administration or in ovo administration. In an embodiment, administered comprises in ovo administration. In an embodiment, administered comprises spray administration. In an embodiment, administered comprises immersion, intranasal, intramammary, topical, or inhalation.

In some aspects the animal is vaccinated in conjunction with administration. The animal may be vaccinated prior to administration of the compositions disclosed herein. The animal may be vaccinated with an coccidiosis vaccine. Coccidiosis vaccines are known in the art, for example, COCCIVAC.

In some embodiments, administration is by way of injection or infusion. In one embodiment, the composition is administered to a cow by way of intra-mammary infusion.

In an embodiment of the method(s), the method does not comprise administration of an antibiotic.

In some embodiments, the compositions or combinations may additionally include one or more prebiotic. In some embodiments, the compositions may be administered along with or may be coadministered with one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric intubation, or broncho-nasal spraying. The compositions disclosed herein may be administered by immersion, intranasal, intramammary, topical, mucosally, or inhalation. When the animal is a bird the treatment may be administered in ovo or by spray inhalation.

Compositions may include a carrier in which the enzyme or peptide is suspended or dissolved. Such carrier(s) may be any solvent or solid or encapsulated in a material that is non-toxic to the inoculated animal and compatible with the enzyme or peptide. Suitable pharmaceutical carriers include liquid carriers, such as normal saline and other non-toxic salts at or near physiological concentrations, and solid carriers, such as talc or sucrose and which can also be incorporated into feed for farm animals. When used for administering via the bronchial tubes, the composition is preferably presented in the form of an aerosol. A dye may be added to the compositions hereof, including to facilitate checking or confirming whether an animal has ingested or breathed in the composition.

When administering to animals, including humans or farm animals, administration may include orally or by injection. Oral administration can include by bolus, tablet or paste, or as a powder or solution in feed, food, or drinking water. Administration may be by ingestion. The method of administration will often depend on the species being feed or administered, the numbers of animals being fed or administered, and other factors such as the handling facilities available and the risk of stress for the animal.

The dosages required will vary and need be an amount sufficient to induce a response or to effect a biological or phenotypic change or response expected or desired. Routine experimentation will establish the required amount. Increasing amounts or multiple dosages may be implemented and used as needed.

The enzyme may be provided in any form suitable for oral administration, such as liquid, solid, powder, gel, etc. The enzyme may be administered alone, or may be formulated in any composition suitable for oral administration. In some embodiments, the composition that is suitable for oral administration is generally recognized as safe for oral administration to an animal. For example, a composition that is suitable for oral administration may contain only ingredients, and amounts of said ingredients, that are generally recognized as safe for oral administration to an animal, and does not contain any ingredients, or amounts of said ingredients, which are not generally recognized as safe for oral administration to an animal. Additionally or alternatively, a composition that is suitable for oral administration contains only ingredients, and amounts of said ingredients, that are allowed, or that are not prohibited, for oral administration to an animal, and does not contain any ingredients, or amounts of said ingredients, that are not allowed, or that are prohibited, for oral administration to an animal.

In some embodiments, the composition comprises an orally acceptable carrier for the enzyme. As used herein, “orally acceptable carrier” includes any physiologically acceptable carrier suitable for oral administration. Orally acceptable carriers include, without limitation, animal feed compositions, aqueous compositions, and liquid and solid compositions suitable for use in animal feed products and/or for oral administration to an animal, including liquid and solid animal feed additives. Suitable carriers are known in the art, and include those described in U.S. Pat. No. 6,780,628.

In some embodiments, the composition is an animal feed. As used herein, the term “animal feed” has its conventional meaning in the field of animal husbandry. For example, animal feed includes edible materials which are consumed by livestock for their nutritional value. Animal feed includes feed rations, e.g., compositions that meet an animal's nutritional requirements, and also include compositions that do not meet an animal's nutritional requirements. In some embodiments, the animal feed is a starter feed, formulated for use during the starter period. In other embodiments, the animal feed is a grower feed, formulated for use during the grower period. In other embodiments the animal feed is finisher feed used in the finishing period.

In specific examples of animal feed embodiment, the amount of enzyme (such as alkaline phosphatase) is at least about 10,000 international units (IU) per U.S. ton of feed, at least about 15,000 international units (IU) per U.S. ton of feed, at least about 20,000 international units (IU) per U.S. ton of feed, at least about 25,000 international units (IU) per U.S. ton of feed, at least about 30,000 international units (IU) per U.S. ton of feed, at least about 35,000 international units (IU) per U.S. ton of feed, at least about 40,000 international units (IU) per U.S. ton of feed, at least about 45,000 international units (IU) per U.S. ton of feed, at least about 50,000 international units (IU) per U.S. ton of feed, at least about 60,000 IU per ton of feed, at least about 70,000 IU per ton of feed, at least about 80,000 IU per ton of feed, at least about 90,000 IU per ton of feed, at least about 100,000 IU per ton of feed, at least about 200,000 IU per ton of feed, at least about 500,000 IU per ton of feed, or at least about 3,000,000 IU per ton of feed or higher.

In specific examples of animal feed embodiment, the amount of enzyme (such as alkaline phosphatase) is at least about 10,000 international units (IU) per kg of feed, at least about 15,000 international units (IU) per kg of feed, at least about 20,000 international units (IU) per kg of feed, at least about 25,000 international units (IU) per kg of feed, at least about 30,000 international units (IU) per kg of feed, at least about 35,000 international units (IU) per kg of feed, at least about 40,000 international units (IU) per kg of feed, at least about 45,000 international units (IU) per kg of feed, at least about 50,000 international units (IU) per kg of feed, at least about 60,000 IU per kg of feed, at least about 70,000 IU per kg of feed, at least about 80,000 IU per kg of feed, at least about 90,000 IU per kg of feed, at least about 100,000 IU per kg of feed, at least about 200,000 IU per kg of feed, at least about 500,000 IU per kg of feed, or at least about 3,000,000 IU per kg of feed or higher.

In some embodiments, the amount of enzyme is in the range of about 25 to about 75 MU/ton (MU=124,000 IU). In some embodiments, the amount of enzyme is at least about 2 MU/ton (240,000 IU/ton or 264 IU/kg).

In other specific examples of animal feed embodiments, the amount of enzyme (such as alkaline phosphatase) is at least about 10 IU/kg feed, at least about 15 IU/kg feed, at least about 20 IU/kg feed, such as at least 20 IU/kg feed, at least at 25 IU/kg feed, at least 30 IU/kg feed, at least 35 IU/kg feed, at least at 40 IU/kg feed, at least at 45 IU/kg feed, at least 50 IU/kg feed, at least 550 IU/kg, or more.

Thus, in some embodiments, the invention provides an animal feed comprising an amount of an enzyme, such as alkaline phosphatase, that is effective to reduce the amount of a detrimental compound, such as ammonia (NH₃) and/or phosphorous, present in or released from animal waste, and/or to increase digestion of phosphorus.

The feed composition may be prepared by methods known in the art. For example, the enzyme can be added to the other feed ingredients at any stage during the manufacturing process, as deemed to be appropriate by those skilled in the art. In one embodiment, the enzyme is provided as a solution, such as a liquid enzyme concentrate that is added to other feed ingredients during the manufacturing process. Alternatively, an enzyme-containing solution is sprayed on to a substantially final form of the animal feed. In another embodiment, the enzyme is provided as a solid composition (such as a powder), such as a solid composition that is added to other feed ingredients during the manufacturing process. Exemplary methods for manufacturing enzyme-containing feed are described in WO 97/41739.

In some embodiments, the composition is other than an animal feed. For example, the composition may be a liquid composition other than an animal feed or a solid composition other than an animal feed. Such compositions may be suitable for direct administration to an animal or may be used as a feed additive (e.g., added to feed prior to feeding) or a feed supplement (including supplements that are diluted with other feed components prior to feeding and supplements that are offered to an animal on a free choice, separate basis). Examples of a liquid composition other than an animal feed include liquid enzyme concentrates, including liquid enzyme concentrates that are typically diluted or combined with other ingredients prior to oral administration to an animal.

In embodiments where the composition is a liquid composition other than an animal feed, such as an enzyme solution, the liquid composition or solution may comprise enzyme (such as alkaline phosphatase) in an amount that is at least about 40,000 international units (IU) per liter of solution, such as at least 40,000 IU/L, at least 50,000 IU/L, at least 60,000 IU/L, at least 70,000 IU/L, at least 80,000 IU/L, at least 90,000 IU/L, at least 100,000 IU/L, at least about 500,000 IU/L, at least about 600,000 IU/L, at least about 700,000 IU/L, at least about 800,000 IU/L, at least about 900,000 IU/L, at least about 1,000,000 IU/L, at least about 2,000,000 IU/L, at least about 5,000,000 IU/L, or at least about 200,000,000 IU/L.

In some embodiments, an amount of liquid composition other than an animal feed, such as about 500 mL or 1000 mL solution, is applied to or combined with an amount of feed, such as to a ton of feed, to arrive at feed formulations with enzyme levels described above. In other embodiments, an amount of liquid composition other than an animal feed is applied to or combined with an amount of feed to prepare an animal feed with an amount of enzyme effective to reduce the amount of a detrimental compound, such as ammonia (NH₃) and/or phosphorous, present in or released from animal waste, and/or to increase digestion of phosphorus.

In embodiments where the composition is a solid composition other than an animal feed, the composition may comprise enzyme (such as alkaline phosphatase) in an amount that is at least about 40,000 IU/kg, such as at least 40,000 IU/kg, at least 50,000 IU/kg, at least 60,000 IU/kg, at least 70,000 IU/kg, at least 80,000 IU/kg, at least 90,000 IU/kg, at least 100,000 IU/kg, at least 120,000 IU/kg, at least 140,000 IU/kg, at least 160,000 IU/kg, at least 180,000 IU/kg, at least 200,000 IU/kg, or at least 60,000,000 IU/kg, or more.

In some embodiments, an amount of a solid composition other than an animal feed is applied to or combined with an amount of feed to arrive at feed formulations with enzyme levels described above. In other embodiments, an amount of solid composition other than an animal feed is combined with an amount of feed to prepare an animal feed with an amount of enzyme effective to reduce the amount of a detrimental compound, such as ammonia (NH₃) and/or phosphorous, present in or released from animal waste, and/or to increase digestion of phosphorus.

In other embodiments, the enzyme is provided in a capsule or tablet form for oral ingestion.

As conventional in the art, the term “IU” or “international unit” refers to an amount of enzyme that will catalyze the transformation of 1 micromole of the substrate per minute under conditions that are optimal for the enzyme. As used herein “MU” (Million Chemgen Units)=120,000 IU. (1 IU=8.33 ChemGen U) Weight equivalents for many enzymes are known in the art and can be determined using standard assays. As known in the art, the selection of buffers and/or substrates can impact the units measured. Standard assays for alkaline phosphatase activity are known in the art. See, e.g., Davidson, Enzyme Microb. Technol. 1: 9-14 (1979); Gonzalez-Gil et al., Marine Ecol. Prog. Ser. 164: 21-35 (1998); Sekiguchi et al., Enzyme Microb. Technol. 49: 171-76 (2011); Simpson et al., Promega Notes 74: 7-9.

In one embodiment of the invention, a dry composition of the invention is present in an amount of more than 100 g per metric ton of complete feed. In one embodiment of the invention, a dry composition of the invention is present in an amount of more than 500 g per metric ton of complete feed.

In one embodiment of the invention, a dry composition of the invention is present in an amount of between 10 g and 30 g per metric ton of concentrated premix. In one embodiment of the invention, a dry composition of the invention is present in an amount of about 20 g per metric ton of concentrated premix.

In one embodiment of the invention, a liquid composition of the invention is present in an amount of less than 100 ml per metric ton of complete feed (liquid). In one embodiment of the invention, a liquid composition of the invention is present in an amount of 50-100 ml per metric ton of complete feed (liquid).

For use in any embodiment of the methods and compositions described herein, the enzyme, such as alkaline phosphatase, can be obtained from a commercial source. Alternatively, the enzyme (including alkaline phosphatase) can be obtained from microorganisms that produce enzymes, such as bacteria, fungi and yeast.

Additionally, the enzyme can be obtained using recombinant technology methods known in the art, by, for example, genetically engineering a host cell to produce an enzyme, e.g., causing transcription and translations of a gene encoding the enzyme. Using known amino acid sequences or known nucleotide sequences encoding those sequences, those skilled in the art can design suitable genes for recombinant expression of the enzyme. Additionally or alternatively, a nucleotide sequence encoding a known enzyme, such as alkaline phosphatase, can be used to probe a DNA library to identify other nucleotide sequences encoding enzymes suitable for use in the methods described herein. As known in the art, such a DNA library can be derived from a defined organism or population of organisms, or can be obtained from natural sources and thus represents DNA from microorganisms that are difficult to culture.

In any embodiment of the methods and compositions described herein, the enzyme, such as alkaline phosphatase, may be expressed by a plant that is used in animal feed. For example, corn could be genetically engineered to express alkaline phosphatase and the resulting genetically modified com product could be used in feed. Production also can be effected with other genetically modified or classically modified systems such as bacteria, e.g., E. coli, Bacillus sp., Lactobacillus; yeast, e.g., Pichia, Yarrow, Saccharomyces, Schizosaccharomyces (e.g., Schizosaccharomyces pomb, Hansenula. Kluyveromyces, Candida), and other fungus, such as Aspergillus, Rhizopus, Tricoderma, Humicola, Penicillium, and Humicola. In specific embodiments, the enzyme, such as alkaline phosphatase, is obtained from Paenibacillus lentus.

In embodiments where a composition comprises a combination of enzymes, the enzymes may be produced individually, by separate organisms, or two or more of the enzymes may be produced by a single organism. For example, a single organism can be recombinantly engineered to produce two or more enzymes by methods known in the art.

As noted above, the invention includes methods for reducing the environmental impact of animal waste, comprising administering to an animal an effective amount of an enzyme that reduces the amount of a detrimental compound present in or released from animal waste. The invention also includes methods for reducing the amount of ammonia in animal waste, comprising administering to an animal an effective amount of an enzyme that reduces the amount of ammonia present in or released from animal waste. The invention also includes methods for reducing the amount of phosphorous present in or released from animal waste, comprising administering to an animal an effective amount of an enzyme that reduces the amount of phosphorous present in animal waste. The enzyme may be administered alone or in any composition described above, including an oral composition, such as animal feed, a liquid composition other than an animal feed, or a solid composition other than an animal feed. The animal may be any animal, including a human or a meat production animal, and may be a healthy animal or an animal suffering from infection or other disease or condition.

In any of these methods, the enzyme may be administered orally, and may be alkaline phosphatase. In any of these methods, the enzyme may be administered orally, and may be the heat tolerant mutant alkaline phosphatase provided herein.

In any of these methods, the animal may be a poultry animal, such as chickens, ducks, turkey, or geese, or a swine animal, such as pigs or hogs. In any of these methods, the enzyme may be administered during one or more of the starter phase, the grower phase, and/or the finisher phase, or at any or all stages.

In any of these methods, the enzyme may be formulated in animal feed, including in a starter feed, a grower feed, or a finisher feed. Alternatively, in any of these methods, the enzyme may be formulated in an animal feed additive.

As noted above, the invention also includes compositions suitable for oral administration to an animal, comprising an effective amount of an enzyme that reduces the amount of a detrimental compound present in or released from animal waste. The invention also includes compositions suitable for oral administration to an animal, comprising an effective amount of an enzyme that reduces the amount of ammonia present in or released from animal waste. The invention also includes compositions suitable for oral administration to an animal, comprising an effective amount of an enzyme that reduces the amount of phosphorous present in or released from animal waste. In any of these compositions, the composition may comprise an orally acceptable carrier for the enzyme. As noted above, the effective amount of enzyme may vary from animal to animal, and from enzyme to enzyme, but readily can be determined by those skilled in the art, as described above and illustrated in the examples.

In any of these compositions, the enzyme may be alkaline phosphatase. In any of these compositions, the enzyme may be heat tolerant mutant alkaline phosphatase provided herein, including as set out in SEQ ID NO: 1, 2 or 3. In any of these compositions, the enzyme may be heat tolerant mutant alkaline phosphatase provided herein, including as set out in SEQ ID NO: 1, 2, 3 or 18-73.

In any of these compositions, the composition may be suitable for administration to birds, humans or non-human mammals. In any of these compositions, the composition may be suitable for administration to poultry, such as chickens, ducks, turkey, or geese, or to swine, such as pigs or hogs.

In any of these compositions, the composition may be an animal feed, such as a starter feed diet or a grower feed diet. Alternatively, in any of these compositions, the composition may be an animal feed additive.

In any embodiments of the invention, one or more additional active ingredients may be employed. In any compositions provided herein, one or more additional active ingredients may be employed. An example of an additional active ingredient is another enzyme, which may have the same or different properties of the enzymes of the invention.

The agents or treatment protocols to be combined or included may include one or more anti-inflammatory drugs or immune suppressants/immune modulators described above. The agents or treatment protocols to be combined or included may be selected from one or more anti-inflammatory, Nonsteroidal anti-inflammatory drug (NSAID), steroid, biologic, antibiotic, or anti-diarrheal agent, including as described above.

Cytokine profiling is typically done using ELISA using lung/tracheal/intestinal homogenates or serum samples. In the absence of reagents needed for ELISA (antibodies for detection of various cytokines) cytokine profiling is done using qRT-PCR on mRNA isolated from RNAlater preserved samples. For example, tissue samples can be collected in RNAlater for cytokine mRNA isolation and/or examination either qualitatively and/or quantitatively by qRT-PCR. Relevant and proinflammatory cytokines include, but are not limited to IL-1, IL-2, IL-12, IL-17, IL-18, IFN-γ, and TNF-α. The key pro-inflammatory cytokines are IL-1, IL-6, and TNF-α. Relevant and anti-inflammatory cytokines include IL-10, IL-22 and IL-12.

The following examples further illustrate the invention, but the invention is not limited to the specifically exemplified embodiments.

As used herein, “subject” includes humans and other mammals, including a human, or a non-human animal, and also birds and fish. A subject includes a bird, poultry, human or non-human animal. Specific examples include bird, poultry, chickens, turkey, dogs, cats, cattle, horse, fish and swine. The chicken may be a broiler chicken, egg-laying or egg-producing chicken. As used herein, the term “poultry” includes domestic fowl, such as chickens, turkeys, ducks, quail, and geese.

The term “treating” or “treatment” of any disease or disorder refers, in one embodiment, to ameliorating the disease or disorder (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter), or both. In a further embodiment, ‘treating’ or ‘treatment’ relates to slowing the progression of the disease. In an aspect, the term “alleviate” or “alleviation” refers to and includes the reduction in the manifestation, extent or severity of a disease or symptom(s) thereof, recognizing that such reduction can serve to reduce pain, suffering, physical or physiological deficit(s), and improve clinical parameters associated with a disease, while not curing or fully eliminating said disease.

The phrase “pharmaceutically acceptable” refers to molecular entities and compositions that are physiologically tolerable and do not typically produce an allergic or similar untoward reaction, such as gastric upset, dizziness and the like, when administered to a human.

The term “therapeutically effective amount” means that amount of a drug, compound, peptide, or pharmaceutical agent that will elicit the biological, physiological, clinical, or medical response of a subject that is being sought by a medical doctor or other clinician. The phrase “therapeutically effective amount” is used herein to include an amount sufficient to prevent, and preferably reduce by at least about 30 percent, more preferably by at least 50 percent, most preferably by at least 90 percent, a clinically significant change in the S phase activity of a target cellular mass, in the enlargement of an organ, in the accumulation of a substrate or protein, in a neurological deficit or impairment, or other feature of pathology such as for example, elevated blood pressure, fever or white cell count, enlargement of the spleen or liver as may attend its presence and activity.

As used herein, the terms “treating”, “to treat”, or “treatment”, include restraining, slowing, stopping, reducing, ameliorating, or reversing the progression or severity of an existing symptom, disorder, condition, or disease. A treatment may be applied prophylactically or therapeutically.

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term “prophylaxis” is related to and encompassed in the term “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

As used herein, “carrier”, “acceptable carrier”, or “pharmaceutical carrier” refers to a diluent, adjuvant, excipient, or vehicle with which the compound is administered. Such carriers can be sterile liquids, such as water and oils, including those of petroleum, animal, vegetable, or synthetic origin; such as peanut oil, soybean oil, mineral oil, sesame oil, and the like. Water or aqueous solution saline solutions and aqueous dextrose and glycerol solutions are preferably employed as carriers, in some embodiments as injectable solutions. Alternatively, the carrier can be a solid dosage form carrier, including but not limited to one or more of a binder (for compressed pills), a glidant, an encapsulating agent, a flavorant, and a colorant. The choice of carrier can be selected with regard to the intended route of administration and standard pharmaceutical practice. See Hardee and Baggo (1998. Development and Formulation of Veterinary Dosage Forms. 2nd Ed. CRC Press. 504 pg.); and E.W. Martin (1970. Remington's Pharmaceutical Sciences. 17th Ed. Mack Pub. Co.).

As used herein, “delivery” or “administration” means the act of providing a beneficial activity to a host. The delivery may be direct or indirect. An administration could be by an oral, nasal, or mucosal route. For example without limitation, an oral route may be an administration through drinking water, a nasal route of administration may be through a spray or vapor, and a mucosal route of administration may be through direct contact with mucosal tissue. Mucosal tissue is a membrane rich in mucous glands such as those that line the inside surface of the nose, mouth, esophagus, trachea, lungs, stomach, gut, intestines, and anus. In the case of birds, administration may be in ovo, i.e. administration to a fertilized egg. In ovo administration can be via a liquid which is sprayed onto the egg shell surface, or an injected through the shell.

The term “solvate” means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease SI), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

A “heterologous” region of a nucleic acid, RNA or DNA, construct is an identifiable segment of RNA or DNA within a larger RNA or DNA molecule that is not found in association with the larger molecule in nature. Thus, when the heterologous region encodes a gene, the gene will usually be flanked by RNA or DNA that does not flank the genomic RNA or DNA in the genome of the source organism.

A “chimeric protein” or “fusion protein” comprises all or (preferably a biologically active) part of a first polypeptide operably linked to a heterologous polypeptide. Chimeric proteins or peptides are produced, for example, by combining two or more proteins having two or more active sites. In a chimeric or fusion protein, a first polypeptide may be covalently attached to an entity which may provide additional function or enhance the use or application of the first polypeptide(s), including for instance a tag, label, targeting moiety or ligand, a cell binding or cell recognizing motif or agent, an antibacterial agent, an antibody, an antibiotic. Exemplary labels include a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. The label may be an enzyme, and detection of the labeled lysin polypeptide may be accomplished by any of the presently utilized or accepted colorimetric, spectrophotometric, fluorospectrophotometric, amperometric or gasometric techniques known in the art. Chimeric protein and peptides can act independently on the same or different molecules or targets, and hence have a potential to provide multiple activities, such as to treat or stimulate immune response against two or more different bacterial infections or infective agents at the same time.

A chimeric protein or fusion protein includes wherein a first heterologous protein of interest is combined with another distinct protein or peptide of interest. A chimeric protein or fusion protein includes wherein a first heterologous protein of interest is combined with a targeting protein or targeting sequence which may direct the first heterologous protein to a particular cell type, a particular cell receptor, or a tissue or region of the body of an animal for instance. A chimeric protein or fusion protein includes wherein a first heterologous protein of interest is combined with a targeting protein or targeting sequence which may direct the first heterologous protein outside of the cell of expression, such as to be expressed or located systemically in an animal, or to the blood local tissues in the animal. A chimeric protein includes wherein a first heterologous protein is combined with a label, tag or enzyme. A tag or label or enzyme may be a functional molecule. A tag or label may be an epitope. A tag or label may be a detectable molecule, protein or other entity. A tag or label may be a fluorescent molecule, a radioactive molecule, etc. Suitable fluorescent molecules are known and available in the art. A fluorescent molecule may be a green fluorescent protein (GFP) for example.

Peptides of and of use in the present invention may include synthetic, recombinant or peptidomimetic entities. The peptides may be monomers, polymers, multimers, dendrimers, concatamers of various forms known or contemplated in the art, and may be so modified or multimerized so as to improve activity, specificity or stability. For instance, and not by way of limitation, several strategies have been pursued in efforts to increase the effectiveness of antimicrobial peptides including dendrimers and altered amino acids (Tam, J. P. et al (2002) Eur J Biochem 269 (3): 923-932; Janiszewska, J. et al (2003) Bioorg Med Chem Lett 13 (21):3711-3713; Ghadiri et al. (2004) Nature 369(6478):301-304; DeGrado et al (2003) Protein Science 12(4):647-665; Tew et al. (2002) PNAS 99(8):5110-5114; Janiszewska, J et al (2003) Bioorg Med Chem Lett 13 (21): 3711-3713). U.S. Pat. No. 5,229,490 to Tam discloses a particular polymeric construction formed by the binding of multiple antigens to a dendritic core or backbone.

In an aspect of the invention, the mutant AP peptides of the invention may be attached to another molecule or may be labeled, including labeled with a detectable label. The label may include or may be selected from radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. The radioactive label can be detected by any of the currently available counting procedures. The isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme may be conjugated to the AP by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase.

In an aspect of the invention, the mutant AP peptides of the invention may be covalently attached to another molecule or may be a fusion protein. Thus, conjugates or fusion proteins of the present invention, wherein the peptide the present invention, or one or more peptide(s) of the invention are conjugated or attached to other molecules or agents further include, but are not limited to peptides conjugated to a cell or pathogen targeting agent or sequence, toxin, immunomodulator, cytokine, cytotoxic agent, or one or more anti-bacterial, anti-parasitic or anti-viral agent or drug.

When administering to animals, including farm animals, administration may include orally or by injection. Oral administration can include by bolus, tablet or paste, or as a powder or solution in feed or drinking water. The method of administration will often depend on the species being treated, the numbers needing treatment, and other factors such as the handling facilities available and the risk of stress for the animal.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. For intravenous, injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The present invention naturally contemplates several means for preparation of mutant AP peptides of the invention, including synthetic methods and/or using known recombinant techniques, and the invention is accordingly intended to cover such recombinant or synthetic preparations within its scope. The determination of the amino acid sequences disclosed herein facilitates the reproduction of the peptides by any of various synthetic methods or any known recombinant techniques. Accordingly, the invention extends to expression vectors comprising nucleic acid encoding the peptides of the present invention for expression in host systems by recombinant DNA techniques, and to the resulting transformed hosts.

In an embodiment, nucleic acid encoding one or more of the peptides of the invention are provided. The invention also relates to a recombinant DNA molecule, recombinant nucleic acid, or cloned gene, or a degenerate variant thereof, preferably a nucleic acid molecule, in particular a recombinant DNA molecule or cloned gene, encoding the amino acid of peptide(s) of the invention. In a particular embodiment, the recombinant DNA molecule, recombinant nucleic acid, or a degenerate variant thereof, preferably a nucleic acid molecule, encodes a peptide(s) of the invention. DNA sequences may be expressed by operatively linking them to an expression control sequence in an appropriate expression vector and employing that expression vector to transform an appropriate unicellular host. Such operative linking of a DNA sequence of this invention to an expression control sequence, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors may depend on the animal or cell type selected for expression and will be available and known to one skilled in the art. Any of a wide variety of expression control sequences—sequences that control the expression of a DNA sequence operatively linked to it—may be used in these vectors to express the DNA sequences of this invention.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, human cells and plant cells in tissue culture.

It will be appreciated that other embodiments and uses will be apparent to those skilled in the art and that the invention is not limited to these specific illustrative examples or preferred embodiments.

### Example 1

### Design and Characterization of Alkaline Phosphatase with Thermostability Improvement

Enzymes, including lysozyme and alkaline phosphatase, are recognized as disruptors of bacterial debris and inflammation. These enzymes reduce inflammation, including by lysing the peptidoglycans and LPS of bacteria, thus deactivating them and thereby bypassing the inflammatory response. An alkaline phosphatase was initially identified and modified from Paenibacillus lentus bacteria and expressed for application and use as a farm animal feed additive. This modified alkaline phosphatase is characterized by having a serine (S) replacement for an aspartic acid (D) in the native bacterial alkaline phosphatase protein sequence and is designated herein as AP1. Although AP1 has only one amino acid change compared to native Paenibacillus lentus alkaline phosphatase (wt AP), it is approximately two times more active than the wt AP. The AP1 alkaline phosphate is described including in U.S. Pat. No. 9,326,535 issued May 3, 2016. The sequence of AP1 is as follows (the serine is in bold and underlined) (SEQ ID NO:4):

ESSNGQSKNLIVLIGDGMGPAQVSAARYFQQHKNNINSLNLDPYYVGQATTYADRGEDGGHIVS
GIVTSSASAGTAFATGNKTYNAAISVSNEDVSRPFASVLEAAELSGKSTGLVTTARITHATPAVY
ASHVRSRDNENAIAFQYLDSGIDVLLGGGESFFVTKEEKGKRNDKNLLPEFEAKGYKVVKTGQS
LKSLSAKDAKVLGLFGGSHIAYVPDRSDETPSLAEMTSKALEILSTNENGFAIMIEGGRIDHAGHA
NDFPTMVQEALDFDEAFKVAIDFAKKDGNTSVVVTADHETGGLSLSRDNIYELNVDLWNKQKN
SSESLVSALNEAKTIADVKKIVSDNTWITDLTNEEAQYILDGDGSSYKREGGYNAVISKRLLVGW
SGHGHSAVDVGVWAYGPIADKVKGQIDNTRIATASAEVLGVDLKKATADLQSKYLYPKFKINR
NKEVLFPAKPLAEALGGKYQAANGTATISGMSGTITVDLNAKKAKLSGNSSSITIDVDNDVLYLP
LTAFSQITGQTLKWDALSERIMLK

Nucleic acid encoding the AP1 (single serine mutation) of SEQ ID NO:4 is as follows:

(SEQ ID NO: 9)
gtgaataaattgttgaagggtttggctatcggggggatcgtgcttgcagttgtgtctgctggaacat
tggctgtggcgaaggagaatgcatctagagctgaaagctcgaacgggcaatcaaaaaatctgatcgt
tctgatcggggacggtatggggcccgcgcaagtttcggctgcgcgttattttcagcaacacaaaaat
gtcatatcgtctccggaatcgtgacgtcctcagcttccgcgggcaccgcatttgcaacgggcaataa
aaatattaacagtctgaacctggatccgtactatgttggacaagcgacgacttatgcagaccgcggc
gaagacggagacatacaatgcggcaataagcgtttccaacgaggatgtatccaggccgttcgcctcg
gacgtattgcttggcggaggggaatccttctttgttacgaaagaggaaaagggcaagcgtaacgata
agtgcttgaagcagccgagctaagcggcaagtcgacaggactggtaacgacagcccgcattacccat
gctactccggggtatacgcttcacatgtgcgaagccgtgataatgagaacgcaattgctttccagta
cttagacagcgggattaaatctgctgccggaattcgaagccaagggatacaaagttgtaaaaacagg
tcaaagtctgaagagcctctcggccaaagacgccaaagtgctgggactgtttggaggctctcatatc
gcttatgtgccagatcgttcggatgaaactccgagtctcgctgaaatgacttcgaaagctttggaaa
tcttatccacaaacgagaatggatttgccatcatgattgagggtggacgcatagatcatgctggaca
cgcgaatgatttccccacgatggtgcaagaagcgctagattttgatgaagcctttaaagtagcgatc
gactttgccaagaaggatggaaatacgtcagtcgtcgtcacagctgatcatgagaccggcggccttt
ccctatcacgggacaacatttacgagttgaacgttgatttgtggaacaagcagaaaaactcctccga
gagcctggtatcggctttgaatgaagccaagacgattgcggatgtaaagaaaatcgtgtcggacaat
acctggatcactgatcttacgaatgaagaggcacagtatattctagacggtgatggctcttcttaca
aacgtgaaggcggttataatgccgtaatctctaagcgtctgctcgtcggctggtctggtcacggaca
ttccgcagtggatgttggagtgtgggcttacggcccaatcgcggataaagtgaaaggacaaatcgac
aatacgaggatcgcgacggcaagtgcggaggtgctcggtgttgatctgaaaaaggctacggctgatc
tccaatcgaagtatctgtatccaaaattcaaaattaaccgcaataaagaagtgctatttccggctaa
gcctttggctgaggcgctaggcggtaagtaccaggcagcaaacggaacggctacaatcagcggcatg
agcgggacaattacagtagatctgaatgcaaagaaggctaaattaagcgggaattcttcctctatta
ccatagatgttgacaatgatgtgctttatcttccgttaacagcatttagccaaattacaggacaaac
cttgaaatgggacgcattgtctgaacggattatgttgaaa

Paenibacillus lentus (formerly designated as B. lentus) has been listed in the AAFCO (Association of American Feed Control Officials) since 1997 as an organism acceptable for use as a direct-fed (live) microorganism in animal feed, and as a source organism for specific feed enzymes (β-mannanase, xylanase, β-glucanase, and others). In 2019, alkaline phosphatase from P. lentus was classified as a GRAS (Generally Recognized As Safe) feed enzyme.

All organisms contain AP enzyme. The AP family of enzymes hydrolyze organic phosphate esters. Isoforms of mammalian AP include intestinal AP (IAL), tissue-nonspecific AP (TNAP), placental AP (PLAP) and germ cells AP (GCAP). Most intestinal AP (IAP) is secreted into the gut lumen inside lipid rafts. The optimum ex vivo or in vitro pH for AP activity is in a non-physiologic alkaline range. There are well-established assays and methods for determining AP enzyme activity, including determinations with non-metabolically relevant substrates. Metabolically relevant substrates include phosphate monoesters; β-glycerol phosphate; phosphorylated nucleotides adenosine triphosphate (ATP) and uridine diphosphate (UDP); microbe-associated molecular patterns (MAMP) using lipopolysaccharide (LPS), flagellin and bacterial unmethylated cytosine-guanosine dinucleotides (CpG); phospholipids; and phosphorylated hydroxyl-AA (Ser, Thr, Tyr) in signaling proteins. While the active site and the protein ‘fold’ is conserved, there is significant diversity in the sequences with greater differences in sequence observed with evolutionary distance. Human PIAP (3MK2) shows 22.7% amino acid sequence identity with E. coli AP (1ALK), 40.6% amino acid sequence identity with shrimp AP (1SHQ), and 50.2% amino acid sequence identity with chicken ALPI. The modified P. lentus AP (AP1) demonstrates limited amino acid sequence identity to AP enzymes from other species and animals, having 23.1% amino acid sequence identity with E. coli AP (1ALK), 17.9% amino acid sequence identity with shrimp AP (1SHQ), 17.5% amino acid sequence identity with chicken APLI, and 19.5% amino acid sequence identity with human PIAP (3MK2). AP1 retains the biochemical properties for AP, the expected subunit structure and conserved chain folding and the conserved active site with ZN++ metal catalysis. It's x-ray crystal structure is conserved with the expected homo dimer structure in comparison to AP from E. coli (1ALK) as well as from shrimp and also with human placental AP 3MK2.

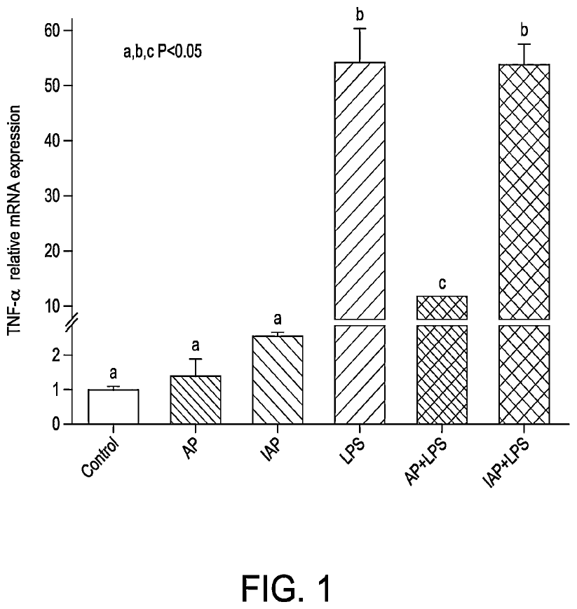

The novel AP1 alkaline phosphatase is very effective at reducing LPS-induced production of pro-inflammatory cytokines in vitro. FIG. 1 depicts a comparison of the AP1 alkaline phosphatase versus calf intestinal alkaline phosphatase (IAP). AP1 is significantly more effective than IAP at reducing inflammation as evaluated by expression of the proinflammatory cytokine TNFα (TNFα mRNA expression).

In order to provide a feed enzyme for ready and successful application and use, value and importance was recognized for developing feed enzymes that survive the feed pelleting process. The criteria for success include: (a) the enzymes should have inherent heat resistance, without requiring coatings or special processing; and (b) the heat resistance should not result in sacrifice of specific activity (turnover rate) at 37-40° C. as encountered in animal's digestive tract. For alkaline phosphatase to become a useful, effective and successful farm animal feed enzyme, having these properties provides for economical production and an active and effective feed enzyme.

In an effort to derive and provide an alternative bacterial alkaline phosphatase with improved heat tolerance, the AP1 alkaline phosphatase was evolved to more heat tolerant protein versions through random mutagenesis and defined mutagenesis. Initial mutants improved heat tolerance. The final heat tolerant alkaline phosphatase molecule, designated APHT, incorporates 14 amino acid changes from the original AP1 alkaline phosphatase and has significantly improved heat tolerance, demonstrating a half-life of 15 minutes at 93-C.

Directed evolution of the alkaline phosphatase gene to heat tolerance (HT)

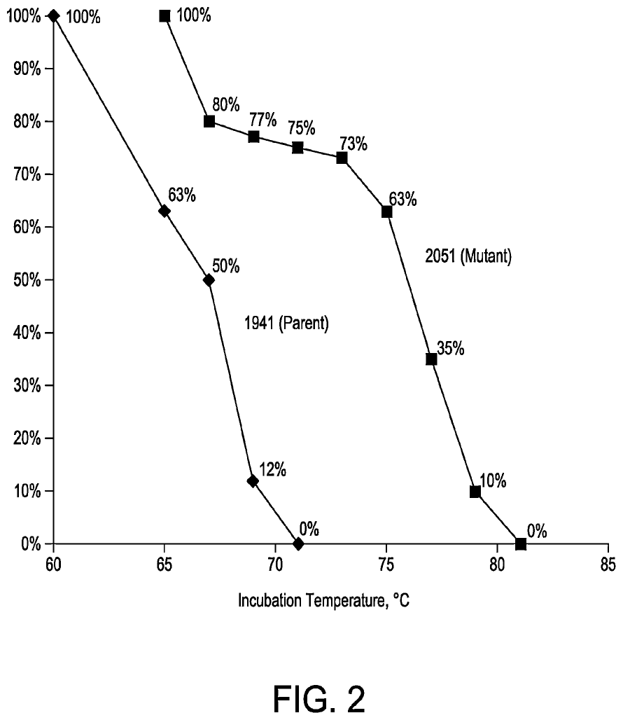

The AP1 alkaline phosphatase was mutated using random mutagenesis to result in an initial altered alkaline phosphatase, designated 2051 mutant, having 10° C. temperature improvement in thermostability, as shown in FIG. 2. This 2051 mutant has four (4) amino acid changes in sequence compared to AP1 and its enzyme sequence is as follows (amino acid changes versus the AP1 sequence are underlined and in bold) (SEQ ID NO:3):

ESSNGQSKNLIVLIGDGMGPAQVSAARYFQQHINNINSLNLDPYYVGQATTYADRGEDGGHIVS
GIVTSSASAGTALATGNKTYNAAISVSNEDVSRPFASVLEAAELSGKSTGLVTTARITHATPAVY
ASHVRNRDNENAIAFQYLDSGIDVLLGGGESFFVTNEEKGKRNDKNLLPEFEAKGYKVVKTGQS
LKSLSAKDAKVLGLFGGSHIAYVPDRSDETPSLAEMTSKALEILSTNENGFAIMIEGGRIDHAGHA
NDFPTMVQEALDFDEAFKVAIDFAKKDGNTSVVVTADHETGGLSLSRDNIYELNVDLWNKQKN
SSESLVSALNEAKTIADVKKIVSDNTWITDLTNEEAQYILDGDGSSYKREGGYNAVISKRLLVGW
SGHGHSAVDVGVWAYGPIADKVKGQIDNTRIATASAEVLGVDLKKATADLQSKYLYPKFKINR
NKEVLFPAKPLAEALGGKYQAANGTATISGMSGTITVDLNAKKAKLSGNSSSITIDVDNDVLYLP
LTAFSQITGQTLKWDALSERIMLK

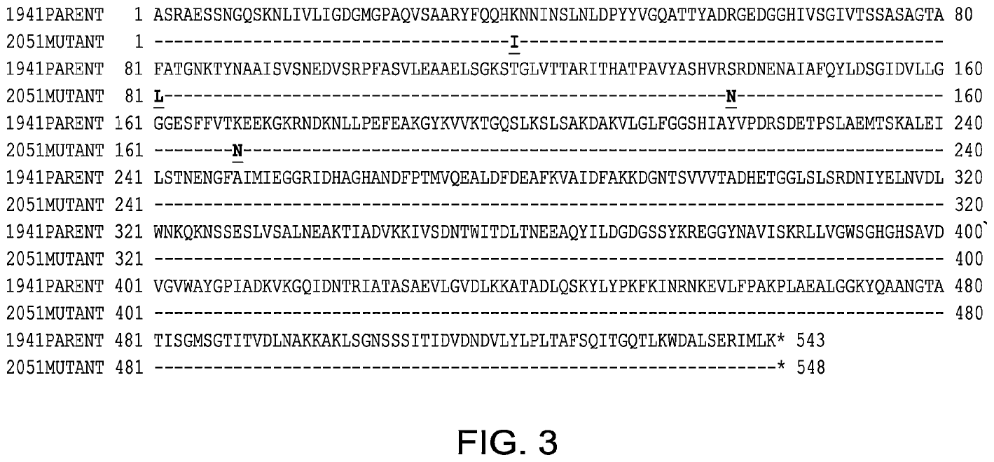

An amino acid comparison of the AP1 versus the 2051 mutant enzyme amino acid sequence is shown in FIG. 3. The amino acid sequences includes a short native leader sequence of ASRA (SEQ ID NO:10) at the N-terminus of the enzyme sequence. The AP1 enzyme with ASRA leader is provided as SEQ ID NO:11. The 2051 mutant enzyme with ASRA leader sequence is provided as SEQ ID NO:12.

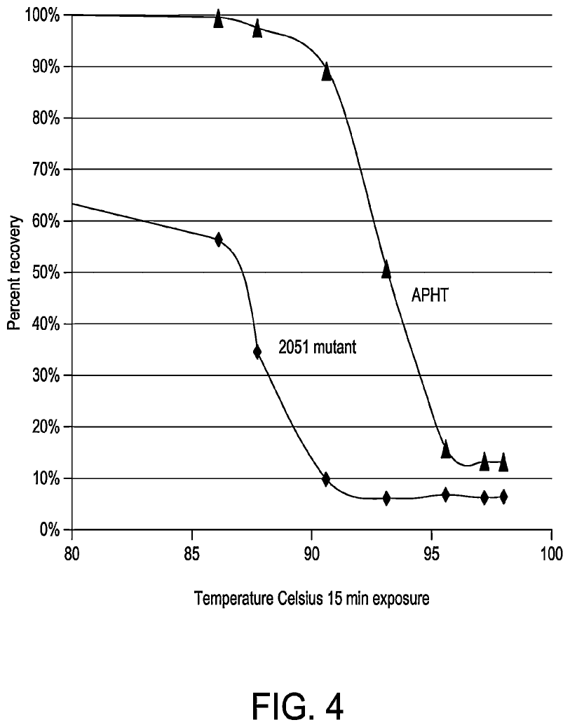

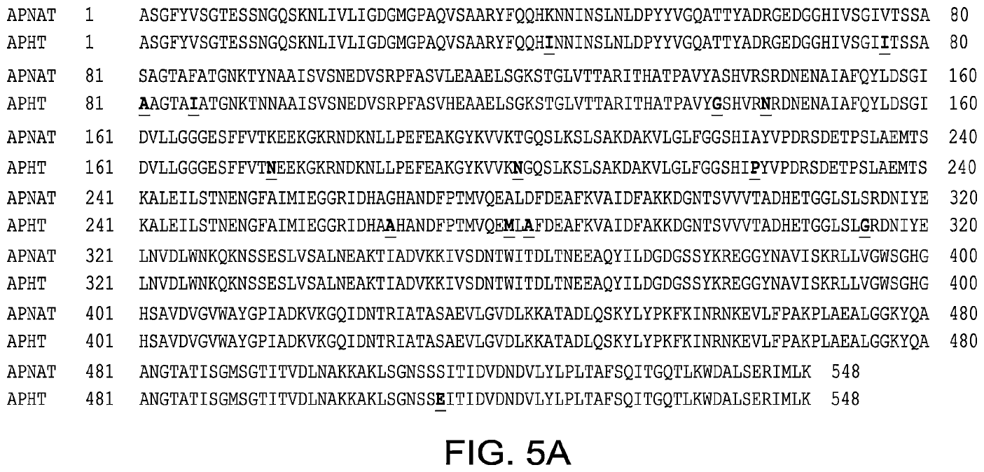

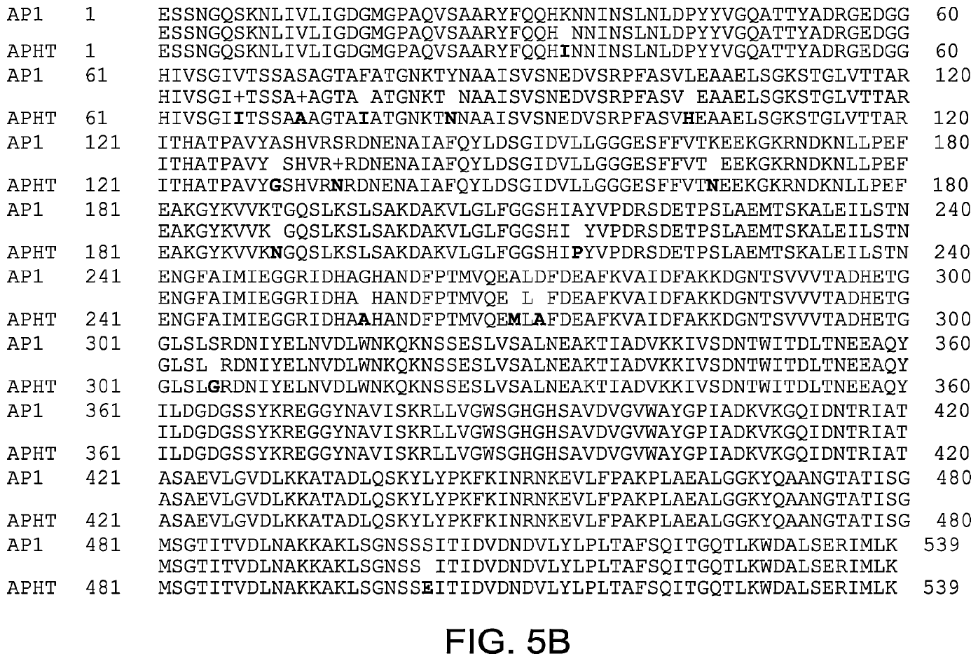

To further improve the thermal stability, a set of synthetic alkaline phosphatase genes where each amino acid was mutated to other possibilities one at a time was constructed and evaluated. Numerous single amino acids changes improving heat tolerance were combined and incorporated into a mutated alkaline phosphatase version yielding the alkaline phosphatase heat tolerant molecule designated APHT. APHT exhibits a 15 minute half-life at 93° C. (FIG. 4), and has a total of 16 amino acid changes from the original AP1 sequence (see FIG. 5A and B). In FIG. 5A, the AP1 with a serine mutation is designated as “AP Native” and is depicted in sequence with a leader sequence ASGFYVSGT (SEQ ID NO:13). The AP1 with leader sequence ASGFYVSGT is provided in SEQ ID NO:14). The APHT polypeptide with leader sequence ASGFYVSGT is provided in SEQ ID NO:15). In FIG. 5B, the AP1 with a serine mutation and without any leader sequence or leader peptide amino acids (SEQ ID NO:4) is aligned and compared with the APHT without any leader sequence or leader peptide amino acids (SEQ ID NO:1). The 16 amino acid changes in APHT are evident. The APHT amino acid sequence (excluding and without any leader sequence and starting with amino acids ESSN) is 97% identical to the sequence of AP1 (excluding and without any leader sequence and starting with amino acids ESSN).

The APHT sequence (SEQ ID NO: 1) is provided below with the amino acid mutations versus the AP1 sequence underlined and in bold.

ESSNGQSKNLIVLIGDGMGPAQVSAARYFQQHINNINSLNLDPYYVGQATTYADRGEDGGHIVSGIITSS
AAAGTAIATGNKTNNAAISVSNEDVSRPFASVHEAAELSGKSTGLVTTARITHATPAVYGSHVRNRDNEN
AIAFQYLDSGIDVLLGGGESFFVTNEEKGKRNDKNLLPEFEAKGYKVVKNGQSLKSLSAKDAKVLGLEGG
SHIPYVPDRSDETPSLAEMTSKALEILSTNENGFAIMIEGGRIDHAAHANDFPTMVQEMLAFDEAFKVAI
DFAKKDGNTSVVVTADHETGGLSLGRDNIYELNVDLWNKQKNSSESLVSALNEAKTIADVKKIVSDNTWI
TDLINEEAQYILDGDGSSYKREGGYNAVISKRLLVGWSGHGHSAVDVGVWAYGPIADKVKGQIDNTRIAT
ASAEVLGVDLKKATADLQSKYLYPKFKINRNKEVLFPAKPLAEALGGKYQAANGTATISGMSGTITVDLN
AKKAKLSGNSSEITIDVDNDVLYLPLTAFSQITGQTLKWDALSERIMLK

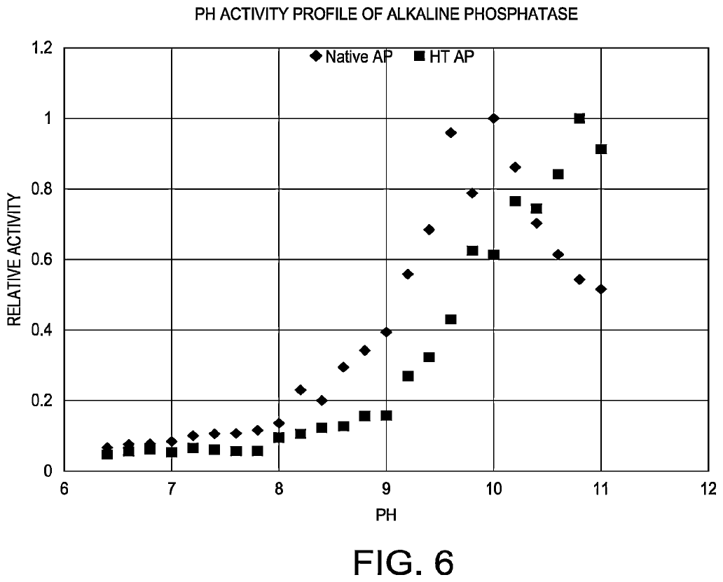

The pH profiles of APHT and AP1 Native are compared in FIG. 6 using fluorescein diphosphate substrate.

### REFERENCES

1. Francia, M. V. and Clewell, D. B. (2002) J. Bacteriol., 184 (18), 187-193.
2. Gryczan, T., Shivakumar, A. G. and Dubnau D. (1980) J. Bacteriol., 141(1), 246-253.
3. Kim, E. C., and Wyckoff (1991) Reaction mechanism of alkaline phosphatase based on crystal structures: Two metal ion catalysis. J. Mol. Boil. 218(2), 449-464.
4. Lalles, J. P (2014) Intestinal Alkaline Phosphatase: novel functions and protective effects. Nutrition Reviews 72 (2): 82-94, 2014
5. Murphy, J. E., Xu, X, and Kantrowitz. E. R. (1993) Conversion of a magnesium binding site into a zinc binding site by a single amino acid substitution in Escherichia coli alkaline phosphatase. J. Biol. Chem. 268 (29) 21497-21500.
6. Davidson D. F. (1979) Measurement of serum alkaline phosphatase. Enzyme Microb. Technol. 1: 9-14.
7. Rose, R. E. (1988) Nucl. Acids Res., 16, 356.
8. Villafane, R., Bechhofer, D. H., Narayanan, C. S. and Dubnau D. (1987) J Bacteriol 169(10):4822-4829.

### Example 2

### Activity Assay of Alkaline Phosphatase: Native (AP1) and APHT

Assays for determining and comparing alkaline phosphatase activity are provided and were utilized to assess the heat tolerant APHT versus the serine mutant AP1 (also designated as Native AP). These evaluations show the ability of APHT to dephosphorylate different substrates and also compare the in vitro activity of the AP1 native mutant versus APHT enzyme for some specific substrates. The APHT enzyme effectively detoxifies LPS, including LPS from various bacterial sources.

### Materials and Methods

### 1. Reagents

p-NPP purchased from VWR catalog number; 97061-486.
ZnCl₂ 0.1 M solution purchased from sigma-aldrich catalog number; 39059-100ML.
Malachite Green assay kit purchased from sigma-aldrich catalog number; MAK-307.
AP Native and HT (Chunghee Lee Notebook AH00950P.1-15)

### 2. Stocks Solutions

1 M DEA Buffer pH 10. Dissolve 10.5 g of diethanolamine in to 100 ml of water. The pH adjust with 1 M hydrochloric acid or 1 M NaOH. Filter sterilize and store at 4 C.
0.5 M Tris Base Buffer pH 10 and 8. Dissolve 6.06 g of Tris in to 100 ml of water. The pH adjust with 1 M hydrochloric acid or 1 M NaOH. Filter sterilize and store at 4 C.
0.1 M Borate Buffer pH 11 and 12. Dissolve 0.62 g Boric acid, 0.44 g NaCl and Sodium tetraborate 0.95 g in 100 ml H₂O. The pH adjust with 1 M hydrochloric acid or 1 M NaOH. Filter sterilize and store at 4 C.
1 M KCl/NaOH Buffer pH 13. Dissolve 7.4 g KCl in 100 ml water. The pH adjust with 1 M NaOH. Filter sterilize and store at 4 C.
1 M MgCl₂, 23.8 g MgCl₂ anhydrous in 2500 mL water. Filter sterilize and store at 4 C
   3. Activity Assay: p-NPP Substrate

Reactions were carried out at 40° C. using 50 mM Tris buffer pH 7 with 50 mM MgCl₂ and at 50° C. using 50 mM DEA buffer pH 10 with 50 mM MgCl₂. Enzyme was thawed, aliquoted in 500 μL vials and freeze at −80° C. For 100 μL volume of reaction, components were mixed in 96-well microplate (Fisher 12565501) following the order in TABLE 1. Every reaction condition was done by triplicated at least.

TABLE 1
Standard reaction components and order of addition

| | | | Volume | | |
|---|---|---|---|---|---|
| Order of | | Stock | added | Final | |
| addition | | concentration | (μL) | concentration | |
| 1 | Water | | Adjusted | | |
| | | | to 100 μL | | |
| 2 | Buffer | 1M | 5 | 50 | mM |
| 3 | MgCl2 | 1M | 5 | 50 | mM |
| 4 | Substrate | 10 X | 10 | | |
| 5 | Enzyme* | | Varies | 2.5 | μg/mL |

After the adding enzyme, the absorption of product was followed at 405 nm over 5 min in a M5 plate reader with intervals of 20 s. Initial rate was determined for each curve and product formed calculated using the extinction coefficient of 18.5 mM⁻¹·cm⁻¹ and a calculated pathlength of 0.5 cm. Initial rates where fitted to Michalis Menten model built in the Kaleidagraph software.

4. pH Profile (Native and HT): p-NPP Substrate

pH effect on enzyme activity was tested at 40 and 50° C. in the corresponding buffer with 50 mM MgCl₂ using p-NPP as substrate in the concentration range 0-5.0 mM. The reaction started with the addition of enzyme (2.5 μg/mL for pH 8 and 0.4 μg/mL for pH 9-13) and the absorption at 405 nm was followed for 5 min. Every reaction condition was done by triplicated at least. The initial rates where fitted to Michalis Menten model built in the software Kaleidagraph.

TABLE 2
Buffers employed for pH profile determination

| Buffer | pH |
|---|---|
| Tris | 8.0 and 9.0 |
| Borate | 11 and 10 |
| DEA | 10.0 |
| KCl/NaOH | 13.0 |

5. Phosphate Assay with Biological Substrates
UDP, ATP, and dAMP (Native and HT)

Phosphatase activity with UDP, ATP and dAMP was assessed by measuring the release of pyrophosphate with the Malachite green reagent (Sigma) at 40° C. and 50° C. Assay consisted of nucleotide phosphate in the concentration range 0-5 mM, 2 μg/mL AP in a final volume of 100 μL. The reaction was initiated in a 96-well microplate with the addition of enzyme and incubated for 5 min. After this time, 10 μL of reaction were mixed with 90 μL of water 25 μL of Malachite green solution. The microplate was shaken for 20 min at 25° C. and absorption was recorded at 620 nm. As standard pyrophosphate solution in water was used in the concentration range of 0-40 μM. Each experiment was done by triplicated at least.

LPS and biological Pi-substrates (Native and HT)

Phosphatase activity with substrates in Table 3 was assessed by measuring the release of pyrophosphate with the Malachite green reagent (Sigma) at 40° C. and 50° C. Assay consisted of substrate at the indicated concentration and 0.4 mg/mL AP in a final volume of 100 μL. The reaction was initiated in a 96-well microplate with the addition of enzyme and incubated for 5 min. After this time, 10 μL of reaction were mixed with 90 μL of water 25 μL of Malachite green solution. The microplate was shaken for 20 min at 25° C. and absorption was recorded at 620 nm. As standard pyrophosphate solution in water was used in the concentration range of 0-40 μM. Each experiment was done by triplicated at least.

TABLE 3
Substrates studied. All substrates were dissolved in water to a final
concentration of 10 or 1 mg/mL (10× of assay concentration).

| | Sigma | Assay |
|---|---|---|
| | Catalog | concentration |
| Compound | number | (mg/mL) |
| Lipopolysaccharides from Escherichia coli | L3137 | 1 |
| O127:B8 | | |
| Lipopolysaccharides from Salmonella enterica | L2262 | 1 |
| serotype typhimurium | | |
| Lipopolysaccharides from Escherichia coli | L2143 | 1 |
| K-235 | | |
| Lipopolysaccharides from Salmonella enterica | L6011 | 1 |
| serotype enteritidis | | |
| Lipopolysaccharides from Salmonella enterica | L6511 | 1 |
| serotype typhimurium | | |
| Lipopolysaccharides from Pseudomonas | L9143 | 1 |
| aeruginosa 10 | | |
| Phosphocholine chloride calcium salt | P0378 | 0.1 |
| tetrahydrate | | |
| Phosphocreatine disodium salt hydrate | P7936 | 0.1 |
| Phytic acid sodium salt hydrate | P8810 | 0.1 |

6. Protein Stability and Tolerance of Malachite-Green Assay to Different Additives (Native and HT) Malachite green assay and AP tolerance to different additives (Table 4) was assessed in 50 mM tris-HCl buffer pH 7.0 and 40 C. Reaction consisted of the indicated concentration of additive with 280 μM UDP and 2 μg/mL AP. The reaction was incubated for 5 min at 40° C. After this time, 10 μL of reaction were mixed with 90 μL of water 25 μL of Malachite green solution. Interference of additives with color development of the Malachite green assay was determined by incubating the additives with Pi (20 μM) in reaction buffer. Every experiment was performed by duplicated at least.

TABLE 4
Additives studied.

| | Assay |
|---|---|
| Additive | concentrations (%) |
| Sucrose, 0-35% | 0, 5, 10, 20, 35 |
| Propylene glycol, 0-10% | 0, 2, 5, 7, 10 |
| Sorbitol, 0-35% | 0, 5, 10, 20, 35 |
| Polyethylene glycol, 0-10% | 0, 2, 5, 7, 10 |
| Hydroxypropyl methylcellulose, 0-2%* | 0, 0.4, 1, 1.4, 2 |
| Potassium sorbate 0 vs 0.1% | 0, 0.1 |
| Sodium benzoate 0 vs 0.1% | 0, 0.1 |

*Viscocity was very high

### Results

The effect of pH on activity of AP1 And APHT at 40 and 50° C. was assessed and determined various pH buffers were utilized and activity was determined.

Activity was assessed at 40° C. in accordance with Table 5:

TABLE 5
40° C.

| | | Vmax IU/mg | | | | KM, μM | | | |
|---|---|---|---|---|---|---|---|---|---|
| Buffer | | Native | stdev | HT | stdev | Native | stdev | HT | stdev |
| Tris | 8 | 161 | 9 | 127 | 8 | 65 | 20 | 46 | 18 |
| Tris | 9 | 2439 | 53 | 2441 | 130 | 315 | 26 | 992 | 137 |
| DEA | 10 | 5752 | 332 | 3870 | 149 | 776 | 125 | 2541 | 189 |
| Borate | 11 | 5210 | 284 | 4309 | 276 | 3341 | 148 | 12204 | 1011 |
| Borate | 12 | 2970 | 79 | 105 | 1819 | 290 | 43 | 498 | 76 |

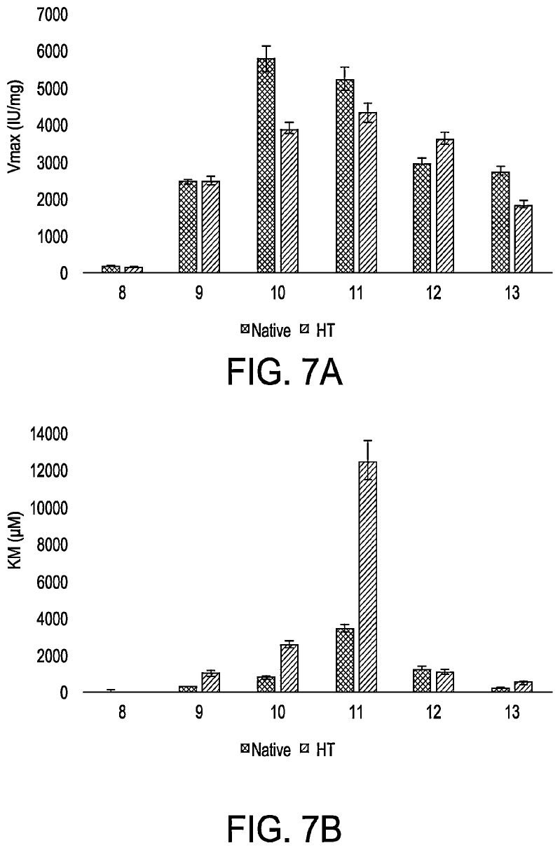

The results in terms of Vmax IU/mg are depicted in FIG. 7A and on KM, M are shown in FIG. 7B.

Activity was assessed at 50° C. in accordance with Table 6:

TABLE 6
50° C.

| | | Vmax IU/mg | | | | KM, μM | | | |
|---|---|---|---|---|---|---|---|---|---|
| Buffer | PH | Native | stdev | HT | stdev | Native | stdev | HT | stdev |
| Tris | 8 | 3752 | 150 | 2848 | 116 | 383 | 55 | 45 | 18 |
| Tris | 9 | 2656 | 78 | 3153 | 137 | 1436 | 97 | 992 | 137 |
| DEA | 10 | 485 | 485 | 6906 | 453 | 2902 | 683 | 2541 | 189 |
| Borate | 11 | 3330 | 95 | 3527 | 59 | 573 | 50 | 12204 | 1011 |
| Borate | 12 | 3042 | 113 | 3179 | 50 | 293 | 42 | 1083 | 129 |
| KCI/NaOH | 204 | 204 | 20 | 109 | 5 | 64 | 35 | 498 | 76 |

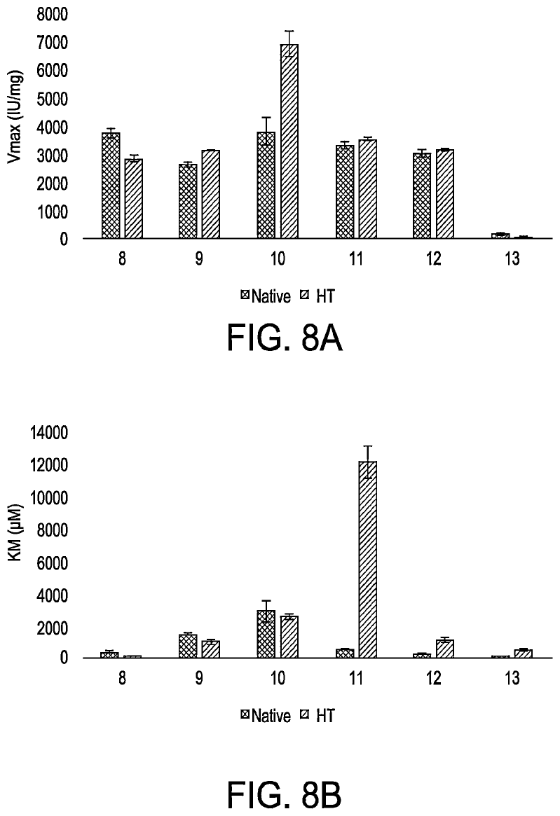

The results in terms of Vmax IU/mg are depicted in FIG. 8A and on KM, M are shown in FIG. 8B.

Enzyme activity with UDP, ATP and dAMP was determined at 40° C. and 50° C.

Activity was assessed at 40° C. in accordance with Table 7:

TABLE 7
40° C.

| | Vmax IU/mg | | | | KM, μM | | | |
|---|---|---|---|---|---|---|---|---|
| Substrate | Native | stdev | HT | stdev | Native | stdev | HT | stdev |
| ATP | 336 | 11 | 358 | 6 | 110 | 17 | 97 | 10 |
| UDP | 447 | 8 | 631 | 8 | 74 | 7 | 93 | 10 |
| dAMP | 356 | 15 | 262 | 12 | 113 | 21 | 165 | 33 |

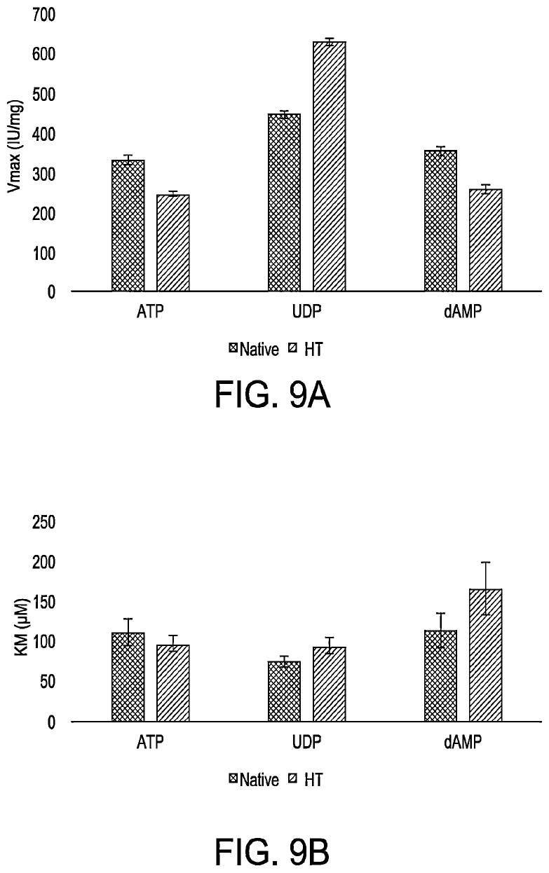

The results in terms of Vmax IU/mg are depicted in FIG. 9A and on KM, M are shown in FIG. 9B.

Activity was assessed at 50° C. in accordance with Table 8:

TABLE 8
50° C.

| | Vmax IU/mg | | | | KM, μM | | | |
|---|---|---|---|---|---|---|---|---|
| Substrate | Native | stdev | HT | stdev | Native | stdev | HT | stdev |
| ATP | 578 | 31 | 380 | 15 | 210 | 43 | 283 | 40 |
| UDP | 608 | 16 | 521 | 16 | 83 | 10 | 121 | 17 |
| dAMP | 730 | 28 | 472 | 58 | 199 | 31 | 623 | 179 |

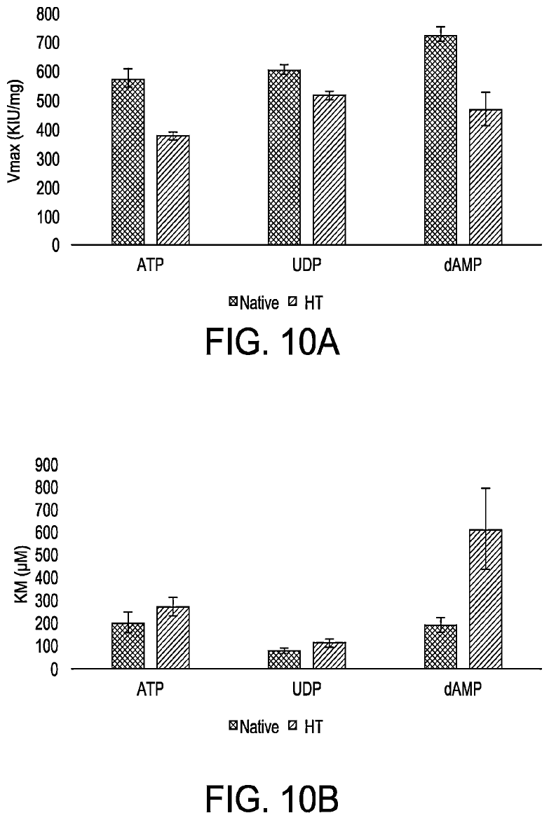

The results in terms of Vmax IU/mg are depicted in FIG. 10A and on KM, M are shown in FIG. 10B.

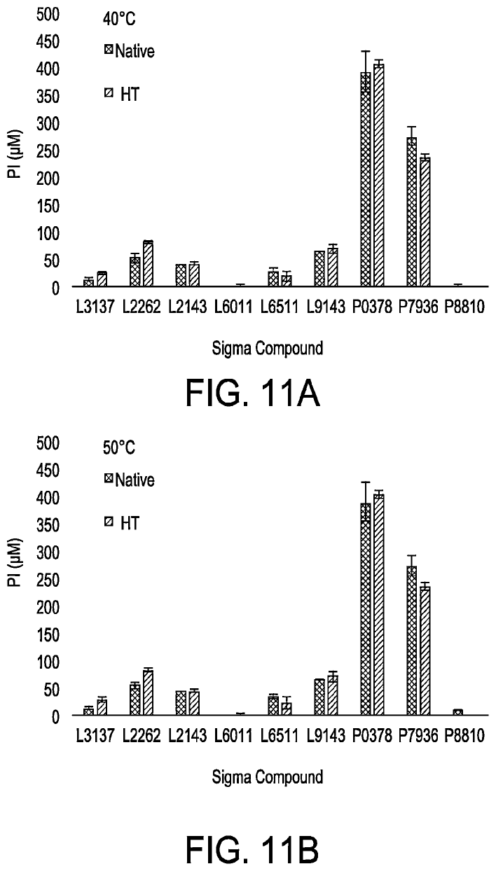

Activity of AP1 (“Native”) and APHT with different LPS and biological substrates was then assessed. Assays were performed in Tris buffer pH 7 (40° C.) and DEA buffer pH 10 (50° C.) for 5 min. The results and assay are detailed in Table 9. Protein concentration in assay 0.4 mg/mL. ND abbreviates not detected. Results are depicted graphically in FIG. 11A (40° C.) and FIG. 11B (50° C.).

TABLE 9

| | Sigma | Activity, 40° C. (IU/mg) | | | | Activity, 50° C. (IU/mg) | | | |
|---|---|---|---|---|---|---|---|---|---|
| Compound | Catalog No | Native | stdev | HT | stdev | Native | stdev | HT | stdev |
| Lipopolysaccharides from | L3137 | 68 | 17 | 135 | 20 | 107 | 14 | 65 | 29 |
| Escherichia coli O127:B8 | | | | | | | | | |
| Lipopolysaccharides from | L2262 | 266 | 38 | 412 | 15 | 167 | 45 | 139 | 35 |
| Salmonella enterica | | | | | | | | | |
| serotype typhimurium | | | | | | | | | |
| Lipopolysaccharides from | L2143 | 211 | 1 | 220 | 12 | 131 | 12 | 105 | 15 |
| Escherichia coli K-235 | | | | | | | | | |
| Lipopolysaccharides from | L6011 | 10 | 3 | 19 | 16 | 37 | 9 | 8 | 10 |
| Salmonella enterica | | | | | | | | | |
| serotype enteritidis | | | | | | | | | |
| Lipopolysaccharides from | L6511 | 161 | 22 | 107 | 49 | 140 | 34 | 98 | 24 |
| Salmonella enterica | | | | | | | | | |
| serotype typhimurium | | | | | | | | | |
| Lipopolysaccharides from | L9143 | 331 | 14 | 355 | 44 | 356 | 9 | 354 | 9 |
| Pseudomonas aeruginosa 10 | | | | | | | | | |
| Phosphocholine chloride | P0378 | 1967 | 184 | 2037 | 33 | 1368 | 122 | 1446 | 142 |
| calcium salt tetrahydrate | | | | | | | | | |
| Phosphocreatine disodium | P7936 | 1370 | 86 | 1184 | 32 | 1225 | 414 | 1187 | 414 |
| salt hydrate | | | | | | | | | |
| Phytic acid sodium salt | P8810 | 30 | 5 | ND | ND | 231 | 93 | 210 | 97 |
| hydrate | | | | | | | | | |

Activity interference and inhibition was evaluated. Interference of various additives with the malachite green assay was first determined. The following additives were evaluated: Sucrose, Propylene glycol, Sorbitol, Polyethylene glycol, Hydroxypropyl methylcellulose, Potassium sorbate and Sodium benzoate. No assay interference was observed with Sucrose, Propylene glycol, Polyethylene glycol, Hydroxypropyl methylcellulose, Potassium sorbate or Sodium benzoate. Sorbitol at an additive concentration of 5% resulted in >50% interference in the assay.

Inhibitory effect of various additives on AP1 and APHT was measured with the malachite green assay after 5 min incubation at 40° C. (280 μM UDP, tris buffer) (data not shown). The following additives were evaluated: Sucrose, Propylene glycol, Sorbitol, Polyethylene glycol, Hydroxypropyl methylcellulose, Potassium sorbate and Sodium benzoate. No assay interference was observed with Hydroxypropyl methylcellulose, Potassium sorbate or Sodium benzoate. Sucrose at 35% showed 10-25% enzyme inhibition for both assays. Propylene glycol at 10% showed 25-50% enzyme inhibition for both assays., Sorbitol at 20-35% showed 10-25% enzyme inhibition for both assays. Polyethylene glycol at an additive concentration of 10% resulted in >50% enzyme inhibition for both assays.

### REFERENCES

Sha Zhu, et al (2009) “The measurement of cyclic nucleotide phosphodiesterase 4 activities via the quantification of inorganic phosphate with malachite green” Analytica Chimica Acta 636(1): 105-110.

### Example 3

### Alkaline Phosphatase HT (Heat Tolerant) Production and Leader Sequences

Microbial alkaline phosphatase (AP), particularly the natural AP from Paenibacillus lentus which is the starting point for the heat tolerant mutants generated herein, is ordinarily produced and expressed in vivo with a leader sequence. All or part of the leader sequence may be cleaved in vivo to provide active enzyme. After such cleavage, a shorter leader peptide sequence may be retained, particularly of 3-10 amino acids. The natural AP from Paenibacillus lentus with the full leader sequence is shown below (the leader sequence MNKLLKGLAIGGIVLAVVSAGTLAVAKENASRA (SEQ ID NO:16) is underlined and in bold):

(SEQ ID NO: 5)
MNKLLKGLAIGGIVLAVVSAGTLAVAKENASRAESSNGQSKNLIVLIGDGMGPAQVSAARYF
QQHKNNINSLNLDPYYVGQATTYADRGEDGGHIVSGIVTDSASAGTAFATGNKTYNAAISVSNE
DVSRPFASVLEAAELSGKSTGLVTTARITHATPAVYASHVRSRDNENAIAFQYLDSGIDVLLGGG
ESFFVTKEEKGKRNDKNLLPEFEAKGYKVVKTGQSLKSLSAKDAKVLGLFGGSHIAYVPDRSDE
TPSLAEMTSKALEILSTNENGFAIMIEGGRIDHAGHANDFPTMVQEALDFDEAFKVAIDFAKKDG
NTSVVVTADHETGGLSLSRDNIYELNVDLWNKQKNSSESLVSALNEAKTIADVKKIVSDNTWIT
DLTNEEAQYILDGDGSSYKREGGYNAVISKRLLVGWSGHGHSAVDVGVWAYGPIADKVKGQID
NTRIATASAEVLGVDLKKATADLQSKYLYPKFKINRNKEVLFPAKPLAEALGGKYQAANGTATI
SGMSGTITVDLNAKKAKLSGNSSSITIDVDNDVLYLPLTAFSQITGQTLKWDALSERIMLK

The AP1 serine mutation alkaline phosphatase with natural full length leader sequence MNKLLKGLAIGGIVLAVVSAGTLAVAKENASRA (SEQ ID NO:16) is shown below (the leader sequence is underlined and in bold; and the serine (s) mutation is underlined and in bold):

AP1 (denoted AP native herein) with D-S mutation with leader sequence

(SEQ ID NO: 6)
MNKLLKGLAIGGIVLAVVSAGTLAVAKENASRAESSNGQSKNLIVLIGDGMGPAQVSAARYF
QQHKNNINSLNLDPYYVGQATTYADRGEDGGHIVSGIVTSSASAGTAFATGNKTYNAAISVSNE
DVSRPFASVLEAAELSGKSTGLVTTARITHATPAVYASHVRSRDNENAIAFQYLDSGIDVLLGGG
ESFFVTKEEKGKRNDKNLLPEFEAKGYKVVKTGQSLKSLSAKDAKVLGLFGGSHIAYVPDRSDE
TPSLAEMTSKALEILSTNENGFAIMIEGGRIDHAGHANDFPTMVQEALDFDEAFKVAIDFAKKDG
NTSVVVTADHETGGLSLSRDNIYELNVDLWNKQKNSSESLVSALNEAKTIADVKKIVSDNTWIT
DLTNEEAQYILDGDGSSYKREGGYNAVISKRLLVGWSGHGHSAVDVGVWAYGPIADKVKGQID
NTRIATASAEVLGVDLKKATADLQSKYLYPKFKINRNKEVLFPAKPLAEALGGKYQAANGTATI
SGMSGTITVDLNAKKAKLSGNSSSITIDVDNDVLYLPLTAFSQITGQTLKWDALSERIMLK

The APHT enzyme sequence has been expressed using alternative leader sequences and at non-native genome locations in bacterial species. Alternative suitable leader sequences—either via constructs or using integrated genome locations may include for example, the Hemicell (endo-1-4-α-mannanases) enzyme leader. The APHT with a full length Hemicell leader sequence MKNLRKKSLSICMAMAMMFSLVTLLGGQDIRAASGFYVSGT (SEQ ID NO:17) is shown below (Leader is in bold and underlined).

APHT Amino acid sequences with Hemicell leader (upcase, cutting at AA):

(SEQ ID NO: 7)
MKNLRKKSLSICMAMAMMFSLVTLLGGQDIRAASGFYVSGTESSNGQSKNLIVLIGDGMGP
AQVSAARYFQQHINNINSLNLDPYYVGQATTYADRGEDGGHIVSGIITSSAAAGTAIATGNKTNN
AAISVSNEDVSRPFASVHEAAELSGKSTGLVTTARITHATPAVYGSHVRNRDNENAIAFQYLDSG
IDVLLGGGESFFVTNEEKGKRNDKNLLPEFEAKGYKVVKNGQSLKSLSAKDAKVLGLFGGSHIP
YVPDRSDETPSLAEMTSKALEILSTNENGFAIMIEGGRIDHAAHANDFPTMVQEMLAFDEAFKVA
IDFAKKDGNTSVVVTADHETGGLSLGRDNIYELNVDLWNKQKNSSESLVSALNEAKTIADVKKI
VSDNTWITDLTNEEAQYILDGDGSSYKREGGYNAVISKRLLVGWSGHGHSAVDVGVWAYGPIA
DKVKGQIDNTRIATASAEVLGVDLKKATADLQSKYLYPKFKINRNKEVLFPAKPLAEALGGKYQ
AANGTATISGMSGTITVDLNAKKAKLSGNSSEITIDVDNDVLYLPLTAFSQITGQTLKWDALSERI
MLK

The Hemicell leader can be cleaved in vivo between the two alanines (AA), which will result in 9 additional amino acids from Hemicell leader being retained in the expressed AP enzyme, or a final leader sequence of ASGFYVSGT (SEQ ID NO:13). These additional leader sequence derived amino acids do not impact APHT activity.

APHT Amino acid sequences without full leader but with retained leader bold and underlined ASGFYVSGT (SEQ ID NO:13) from Hemicell after cleavage at AA:

(SEQ ID NO: 8)
ASGFYVSGTESSNGQSKNLIVLIGDGMGPAQVSAARYFQQHINNINSLNLDPYYVGQATTYADR
GEDGGHIVSGIITSSAAAGTAIATGNKTNNAAISVSNEDVSRPFASVHEAAELSGKSTGLVTTARI
THATPAVYGSHVRNRDNENAIAFQYLDSGIDVLLGGGESFFVTNEEKGKRNDKNLLPEFEAKGY
KVVKNGQSLKSLSAKDAKVLGLFGGSHIPYVPDRSDETPSLAEMTSKALEILSTNENGFAIMIEG
GRIDHAAHANDFPTMVQEMLAFDEAFKVAIDFAKKDGNTSVVVTADHETGGLSLGRDNIYELN
VDLWNKQKNSSESLVSALNEAKTIADVKKIVSDNTWITDLTNEEAQYILDGDGSSYKREGGYNA
VISKRLLVGWSGHGHSAVDVGVWAYGPIADKVKGQIDNTRIATASAEVLGVDLKKATADLQSK
YLYPKFKINRNKEVLFPAKPLAEALGGKYQAANGTATISGMSGTITVDLNAKKAKLSGNSSEITI
DVDNDVLYLPLTAFSQITGQTLKWDALSERIMLK

There are alternative leader sequences from bacteria, including P. lentus which may be utilized and suitable. In each instance the leader sequence does not significantly alter the AP enzyme activity or its heat tolerance attributed by the amino acid mutations in its sequence. The leader from xyloglucanase, hemicell 70 (HC70), xylanase, and s-layer protein are all alternative leader sequences that can be utilized for protein expression and/or secretion.

Alternative amino acid mutations for heat tolerant Alkaline Phosphatase.

Alternative amino acid changes at the amino acid substitution sites in the APHT sequence were identified in the mutant screening for single amino acid changes generating increased het tolerance.

The APHT sequence is provided below with the amino acid mutations versus the AP1 sequence underlined and in bold and alternative amino acids also providing heat tolerance shown below each altered amino acid. The alternative amino acids providing heat tolerance were identified in additional mutational studies, including random mutagenesis to identify amino acid substitutions providing improvements in heat tolerance. These provide additional variant mutant heat tolerant alkaline phosphatase sequences, such as set out below and in the AP mutant grouped or consensus variant sequence (SEQ ID NO:2):

```
ESSNGQSKNLIVLIGDGMGPAQVSAARYFQQHINNINSLNLDPYYVGQATTYADRGEDGGHIVS
                                                                L
GIITSSAAAGTAIATGNKTNNAAISVSNEDVSRPFASVHEAAELSGKSTGLVTTARITHATPAV
                                                                            F
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLGRDNIYELNVDLWNKQ
                        V
                        T
                        E
                          E
                                                                                                A
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
```

Particular alternative variant AP sequences can be derived based on the above variation options in amino acid sequences to derive alternative AP which are heat tolerant can thus be selected from:

#1 (SEQ ID NO: 18):
ESSNGQSKNLIVLIGDGMGPAQVSAARYFQQHLNNINSLNLDPYYVGQATTYADRGEDGGHIVS
GIITSSAAAGTAIATGNKINNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#2 (SEQ ID NO: 19):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#3 (SEQ ID NO: 20):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLAFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#4 (SEQ ID NO: 21):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#5 (SEQ ID NO: 22):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#6 (SEQ ID NO: 23):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#7 (SEQ ID NO: 24):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLARDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#8 (SEQ ID NO: 25):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#9 (SEQ ID NO: 26):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLAFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#10 (SEQ ID NO: 27):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#11 (SEQ ID NO: 28):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLEGGSHIPYVPDRSDETPSLAEMTSKALEILSTNENGFAIMIEGGRIDHA
AHANDFPTMVQEELAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQI TGQTLKWDALSERIMLK
#12 (SEQ ID NO: 29):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#13 (SEQ ID NO: 30):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#14 (SEQ ID NO: 31):
ESSNGQSKNLIVLIGDGMGPAQVSAARYFQQHLNNINSLNLDPYYVGQATTYADRGEDGGHIVS
GIITSSAAAGTAIATGNKINNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#15 (SEQ ID NO: 32):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELEFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#16 (SEQ ID NO: 33):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#17 (SEQ ID NO: 34):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLAFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#18 (SEQ ID NO: 35):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#19 (SEQ ID NO: 36):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#20 (SEQ ID NO: 37):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLEFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#21 (SEQ ID NO: 38):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLEFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#22 (SEQ ID NO: 39):
ESSNGQSKNLIVLIGDGMGPAQVSAARYFQQHINNINSLNLDPYYVGQATTYADRGEDGGHIVS
GIITSSAAAGTAIATGNKINNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#23 (SEQ ID NO: 40):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#24 (SEQ ID NO: 41):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#25 (SEQ ID NO: 42):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLAFDEAFKVAIDFAKKDGNTSVVVTADHETGGLSLARDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#26 (SEQ ID NO: 43):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#27 (SEQ ID NO: 44):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELAFDEAFKVAIDFAKKDGNTSVVVTADHETGGLSLARDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#28 (SEQ ID NO: 45):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#29 (SEQ ID NO: 46):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#30 (SEQ ID NO: 47):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELEFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#31 (SEQ ID NO: 48):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#32 (SEQ ID NO: 49):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLAFDEAFKVAIDFAKKDGNTSVVVTADHETGGLSLARDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#33 (SEQ ID NO: 50):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#34 (SEQ ID NO: 51):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#35 (SEQ ID NO: 52):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#36 (SEQ ID NO: 53):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVHEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#37 (SEQ ID NO: 54):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLEFDEAFKVAIDFAKKDGNTSVVVTADHETGGLSLARDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#38 (SEQ ID NO: 55):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLEFDEAFKVAIDFAKKDGNTSVVVTADHETGGLSLARDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#39 (SEQ ID NO: 56):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLEFDEAFKVAIDFAKKDGNTSVVVTADHETGGLSLARDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#40 (SEQ ID NO: 57):
ESSNGQSKNLIVLIGDGMGPAQVSAARYFQQHINNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#41 (SEQ ID NO: 58):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#42 (SEQ ID NO: 59):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#43 (SEQ ID NO: 60):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVEEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLAFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#44 (SEQ ID NO: 61):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSINENGFAIMIEGGRIDHA
AHANDFPTMVQEELAFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#45 (SEQ ID NO: 62):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#46 (SEQ ID NO: 63):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#47 (SEQ ID NO: 64):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLEFDEAFKVAIDFAKKDGNTSVVVTADHETGGLSLGRDNIYELNVDLWNKQ
KNSSESLVSALNEAKTIADVKKIVSDNTWITDLTNEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#48 (SEQ ID NO: 65):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELEFDEAFKVAIDFAKKDGNTSVVVTADHETGGLSLGRDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#49 (SEQ ID NO: 66):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVEEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#50 (SEQ ID NO: 67):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#51 (SEQ ID NO: 68):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQETLEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#52 (SEQ ID NO: 69):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELEFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#53 (SEQ ID NO: 70):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEMLAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#54 (SEQ ID NO: 71):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEVLAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#55 (SEQ ID NO: 72):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMISKALEILSTNENGFAIMIEGGRIDHA
AHANDFPTMVQETLAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK
#56 (SEQ ID NO: 73):
ESSNGQSKNLIVLIGDGMGPAQVSAARYFQQHLNNINSLNLDPYYVGQATTYADRGEDGGHIVS
GIITSSAAAGTAIATGNKTNNAAISVSNEDVSRPFASVFEAAELSGKSTGLVTTARITHATPAV
YGSHVRNRDNENAIAFQYLDSGIDVLLGGGESFFVTNEEKGKRNDKNLLPEFEAKGYKVVKNGQ
SLKSLSAKDAKVLGLFGGSHIPYVPDRSDETPSLAEMTSKALEILSTNENGFAIMIEGGRIDHA
AHANDFPTMVQEELAFDEAFKVAIDFAKKDGNTSVVVTADHETGGLSLARDNIYELNVDLWNKQ
KNSSESLVSALNEAKTIADVKKIVSDNTWITDLINEEAQYILDGDGSSYKREGGYNAVISKRLL
VGWSGHGHSAVDVGVWAYGPIADKVKGQIDNTRIATASAEVLGVDLKKATADLQSKYLYPKFKI
NRNKEVLFPAKPLAEALGGKYQAANGTATISGMSGTITVDLNAKKAKLSGNSSEITIDVDNDVL
YLPLTAFSQITGQTLKWDALSERIMLK

### Example 4

### In Vitro and In Vivo Evaluations of Alkaline Phosphatase HT as a Feed Additive Candidate

Intestinal inflammation result from many factors including dietary antinutritional components or abrupt changes of the intestinal environment resulting in dysbiosis. The purpose of our studies to develop a heat tolerant alkaline phosphatase HT AP is to generate an alternative feed additive candidate able to mitigate the effect of bacterial infections and bacteria-mediated inflammation, including Gram-negative related enteric inflammation. Multiple in vitro and in vivo assessment methodologies have shown that APHT can reduce the negative inflammatory impact related to the presence of bacterial lipopolysaccharides (LPS) in the intestinal content. APHT reduced the production of pro-inflammatory cytokines and improved intestinal permeability markers. Lower inflammation resulted in improved performance parameters and more efficient nutrient utilization in poultry and swine.

### Materials and Methods and Results

In vitro dephosphorylation assay: Inorganic phosphorus release from different commercial lipopolysaccharide sources. Dephosphorylation of LPS depends on the type and structure of LPS and differences can be seen among gram negative bacterial species and strains, including depending on allosteric effects of the 0 antigen and on the accessibility of the P on lipid A, as well as the presence of diester bonds not hydrolyzed by AP.

Various commercially available LPS sources were assessed as follows in Table 10:

TABLE 10

| | | Specific |
|---|---|---|
| | | activity, |
| Lipopolysaccharide source | Sigma # | mIU/mg |
| Escherichia coli O127:B8 | L3137 | 13.5 ± 2.0 |
| Escherichia coli K-235 | L2143 | 22.0 ± 1.2 |
| Salmonella enterica serotype Typhimurium | L2262 | 41.2 ± 1.5 |
| Salmonella enterica serotype Typhimurium | L6511 | 10.7 ± 4.9 |
| Salmonella enterica serotype Enteritidis | L6011 | 1.9 ± 1.6 |
| Pseudomonas aeurginosa | L9143 | 35.5 ± 4.4 |

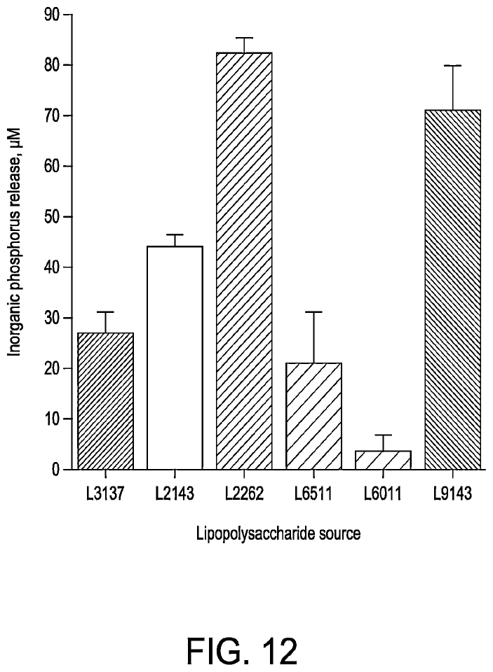

The inorganic phosphorous release (μM) from each of these sources by the heat tolerant HTAP was determined and is shown in FIG. 12. A comparison of phosphate (Pi) release from these sources with native AP showed that the HTAP performed similarly in Pi release, and in some instances demonstrated greater Pi release, compared to native AP (data not shown). The inorganic phosphorous release (μM) was also assessed from the metabolically relevant compounds phosphocholine, phosphocreatine and phytic acid and compared with native AP. The heat tolerant HTAP was comparably active vs native AP in Pi release from each of compounds phosphocholine, phosphocreatine and phytic acid (data not shown).

Dephosphorylation of substrates UDP, ATP and dAMP was also assessed and determined. The results and kinetic constants for phosphatase activity are depicted below in TABLE 11:

| Substrate | Vₘₐₓ (min⁻¹) | K_{M} (μM) |
|---|---|---|
| UDP | 7.5 ± 0.1 | 166 ± 14 |
| ATP | 6.0 ± 0.2 | 399 ± 68 |
| dAMP | 7.9 ± 0. | 242 ± 27 |

In vitro assessment of immunological properties of APHT: Stimulation of primary porcine alveolar macrophages with a selected Gram-negative bacteria LPS.

Salmonella Typhimurium LPS activity was evaluated in porcine alveolar macrophages in the presence of APHT and in the presence of Sigma commercially available intestinal alkaline phosphatase (Sigma-IAP).

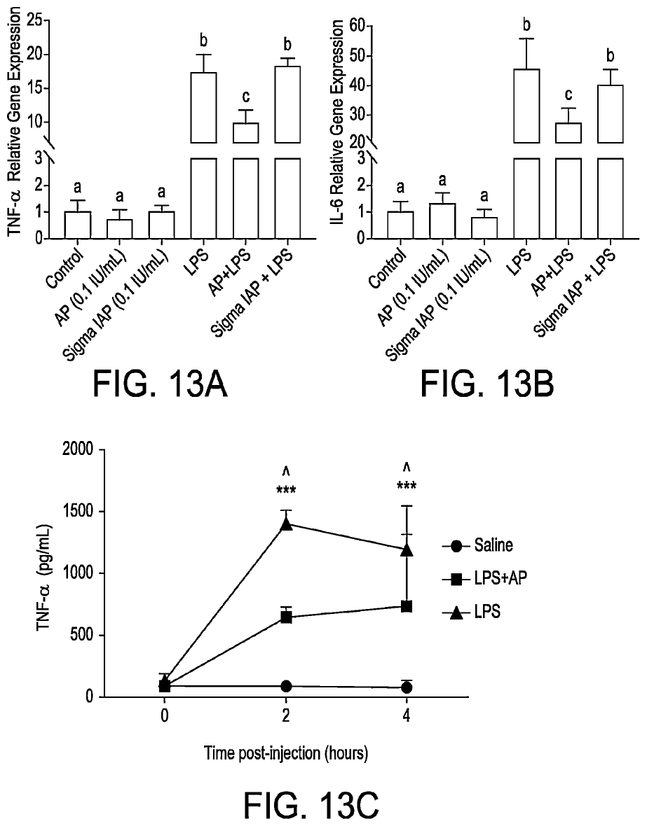

Determination of APHT effect on pro-inflammatory cytokine production and tight junction protein gene expression in swine. Relative gene expression of TNF-α and of IL-6 was determined to evaluate LPS effects in RAW 264.7 murine macrophages in vitro (FIG. 13A and B). Plasma concentration of TNF was also determined (FIG. 13C). The study was conducted using Salmonella bacteria derived LPS. A similar study was conducted with E. coli derived LPS with comparable results (data not shown). In each instance the mutant AP reduced inflammatory cytokine expression, and with significantly improved reduction of cytokine expression in comparison to Sigma purchased IAP. Further experiments evaluated inflammatory marker cytokines IL-6 and IL-10 (data not shown). The mutant AP reduced LPS-mediated cytokine expression of IL-6 and also of IL-10, again with either E. coli LPS or Salmonella LPS, and at greater reduction compared to Sigma IAP control. The APHT enzyme is comparable or more effective at reducing the inflammatory effects of LPS versus calf IAP.

Dose titration study in weaned pigs: Evaluation of the impact of different concentrations of APHT on performance variables. Pig growth performance is shown below in Table 12:

TABLE 12

| Overall 42-d | Control | Alpine HT | S.E.M. | P-value |
|---|---|---|---|---|
| ADFI, kg | 0.705 | 0.710 | 0.012 | 0.94 |
| ADG, kg | 0.413 | 0.452 | 0.010 | 0.31 |
| FCR kg/kg | 1.633 | 1.581 | 0.015 | 0.01 |
| G:F kg/kg | 0.614 | 0.635 | 0.006 | 0.01 |
| Final BW, kg | 24.08 | 24.83 | 0.48 | 0.49 |

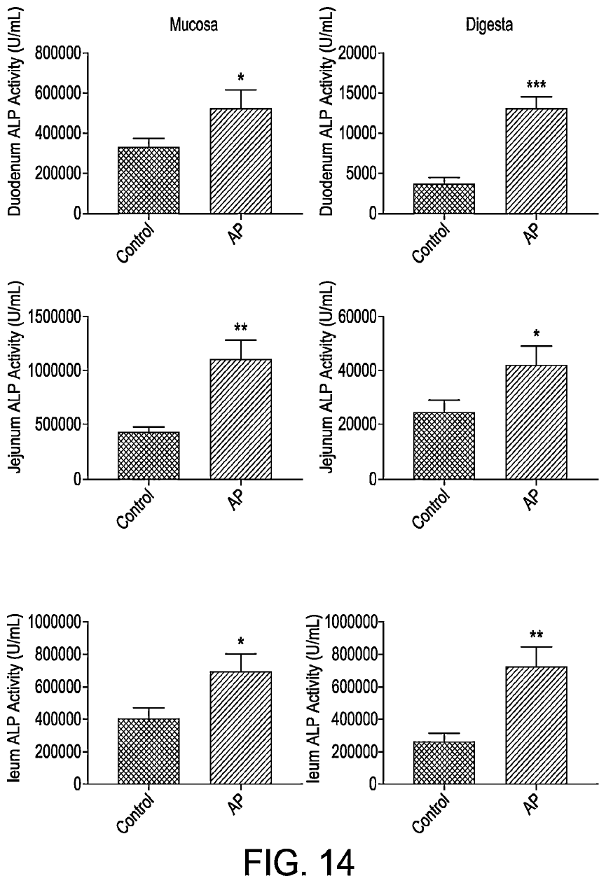

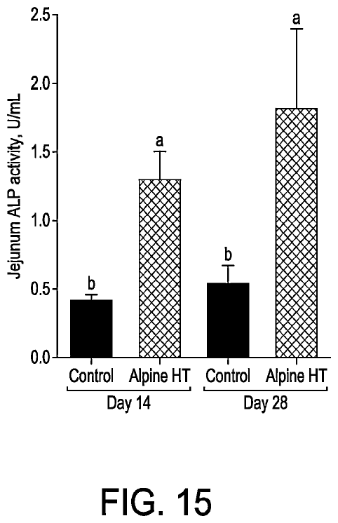

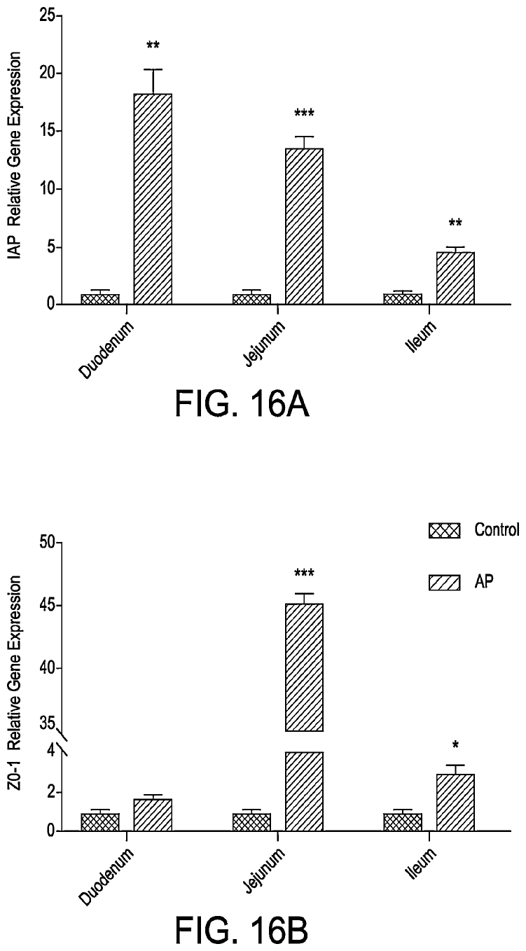

Evaluation of intestinal enzyme activity in broilers and pigs. Alkaline phosphatase activity was evaluated in the small intestine of animals fed APHT. Swine and Broiler chickens were evaluated. These animals ordinarily have natural wild type alkaline phosphatase. APHT was provided additionally as an externally administered AP peptide enzyme in the animal's feed. The alkaline phosphatase present in swine fed a control diet or supplemented with AP in the muosa and the digesta are depicted in FIG. 14. AP activity in the jejunum of broilers fed a control diet or supplemented with AP is depicted in FIG. 15. Intestinal alkaline phosphatase gene expression and ZO-1 relative gene expression was evaluated and assessed in the duodenum, jejunum and ileum of swine fed a control diet or fed diet supplemented with APHT (FIGS. 16A and 16B respectively).

Effect of Alpine HT supplementation on carcass and cuts yield in broilers was determined Broiler carcass meat yield in control versus animals fed APHT (also denoted Alpine HT) is shown in Table 13:

TABLE 13

| | Wings, | Breast, | Tender, | Leg quarters, | Rack, | Carcass, | Carcass | |
|---|---|---|---|---|---|---|---|---|
| Treatment | g | g | g | g | g | g | FCR | Diff |
| Control | 202.2 | 560.4 | 121.8 | 573.7 | 558.7 | 2017 | 2.189 | |
| Alpine HT | 211.6 | 592.9 | 129.1 | 606.3 | 589.2 | 2129 | 2.132 | −5.7 |

Preliminary values of net energy using indirect calorimetry and body composition measurements in broilers. Indirect calorimetry of broilers was assessed and the extra ME with APHT (denoted Alpine HT) is shown in Table 14:

TABLE 14

| | Starter | Grower | Finisher | Overall |
|---|---|---|---|---|
| Extra ME with | 412 | 132 | 226 | 257 |
| Alpine HT, kcal/kg | | | | |

The DEXA body composition of broilers at day 28 on control versu APHT (Alpine HT) fed animals was assessed and is shown below in Table 15:

TABLE 15

| Treatment | Lean mass, g | Protein Mass, g | Fat Mass, g | Energy, kcal |
|---|---|---|---|---|
| Control | 1,268 | 245 | 166 | 2,770 |
| Alpine HT | 1,331 | 258 | 165 | 2,859 |

### Example 5

### Immunoprotective Effects of Lipopolysaccharide Detoxification by a Novel Microbial-Derived Alkaline Phosphatase

Lipopolysaccharide (LPS) is a major outer cell wall component of Gram-negative bacteria, including Escherichia coli and Salmonella enterica, which have important health and economic consequences in animal agriculture. Lipopolysaccharides are a large group of molecules comprised of lipid and polysaccharides, with great variety between bacterial species and strains. LPS is structurally organized into an outer polysaccharide region and an inner region containing lipid A. Lipid A is the most conserved region of LPS molecules and invokes a strong immune response in vertebrates that is dependent on its phosphorylation site. Alkaline phosphatase removes phosphate (the 1-phosphate group) from lipid A in LPS to generate monophosphoryl lipid A (MLPA), which is less active and inflammatory. MLPA has a lower toxicity but the same immunogenicity as LPS. PLPA also increases the ability of antigen-presenting cells to uptake antigens and activate the MHC in effector cells, as well as increasing the ability of M cells to uptake antigen. AP-mediated phosphate release from LPS can be measured in vitro in evaluation of AP. LPS is a recognized endotoxin and can lyse bacterial cell walls and alter membrane vesicle trafficking. LPS stimulates toll-like receptors, which are present on cells that contribute to inflammatory responses and, via downstream signaling pathways, results in the release of pro- and anti-inflammatory mediators to assist with pathogen clearance and tissue repair. Lipid A binds to toll like receptors including TLR-4 thereby inducing inflammatory response. The inflammatory response is dependent on the phosphorylation status of stimulators like LPS therefore dephosphorylation with AP can reduce local and systemic inflammation. AP is implicated in reducing the toxic effects from gram-negative bacteria and eliciting or improving potential immunological benefits of MLPA.

During weaning, piglets are subjected to numerous psychological and environmental stressors including mixing, lower ambient temperature and change in diet. Due to the piglets' immature digestive and immune system, weaning is associated with growth plateaus, increased incidence of diarrhea, increased bacterial translocation and endotoxin load, and increased mortality. Increased concentrations of LPS in circulation perpetuate post-wean syndrome and drive further gastrointestinal tract (GIT) morphological and digestive perturbations. Weaning is also characterized by reductions in intestinal alkaline phosphatase (IAP), which has biological roles including detoxifying LPS in the intestinal lumen and maintaining an alkalotic pH to modulate gut microbiota. IAP is a homodimeric enzyme that catalyzes the release of an inorganic phosphate from the lipid A moiety of LPS, resulting in the considerably less immunostimulatory product, monophosphoryl lipid A (MPLA).

As predicted by the structural similarity, the biochemical properties of the B. lentus AP are consistent with the published characteristics of alkaline phosphatases that have been studied extensively. Key properties are a high pH activity profile, and a very broad substrate specificity for phosphate monoesters. What is very convenient about AP is that the substrate specificity extends to may artificial substrates that makes it easy to assay and track the presence of the enzyme. This is why AP is frequently used in various biochemical diagnostic kits and enzyme coupled immunological assays.

Based on IAP's ability to reduce the inflammatory effects of LPS in the GIT and the difficulty in creating a viable commercial product from a mammalian-derived enzyme; a microbe was engineered to produce heat tolerant alkaline phosphatase (APHT, also designated as MAP). To test the ability of APHT/MAP to detoxify LPS and assess whether it will detoxify LPS similarly to IAP, RAW264 macrophages and primary porcine alveolar macrophages were treated with vehicle, IAP, MAP, E. coli or S. enterica LPS, LPS that had been pre-incubated with MAP (MPLA), or LPS that had been pre-incubated with IAP (iMPLA). In support of the hypothesis, cells treated with MPLA had significantly lower TNFα, IL-6, IL-1β, and IL-10 gene expression as compared to cells treated with LPS alone. Furthermore, novel findings indicate an increased efficacy for MAP to detoxify LPS as compared to IAP. MPLA treated cells had significantly lower levels of pro- and anti-inflammatory cytokine production as compared to iMPLA-treated cells. We then determined the serum cytokine profiles of piglets 2 days post-wean after being challenged with saline, MPLA, or LPS for four hours. Piglets i.p. injected with MPLA were more active and ate more over the time period than LPS injected piglets. Furthermore, MPLA piglets had altered serum cytokine profiles indicating an ameliorated immune response to the challenge.

We investigated the effects of exogenous MAP supplementation on post-wean syndrome in piglets on a standard phase 2 diet. Post-weaning diarrhea (PWD) is part of the post-weaning syndrome and represents one of the most significant economic wastes for the pig industry. PWD is characterized by frequent discharge of watery feces during the first two weeks post-weaning. One of the essential functions of the small intestine is nutrient digestion and absorption. This includes secretion of fluids and electrolytes from crypt cells and nutrient absorption via enterocytes from the intestinal brush-border_{(13, 14)}. Weaning reduces the small intestines' capacity for net absorption of fluid and electrolytes and leads to malabsorption of nutrients_{15,16}. Following weaning, a net secretory condition can occur when the fluid and electrolyte influx into the GIT lumen exceeds the efflux into the blood, and this contributes to the pathology of PWD_{13,14}. PWD is also associated with increased fecal shedding of a significant number of enterotoxigenic E. coli serotypes that proliferate in the small intestine_{17,18}. We found that adding 4,000 IU/kg body weight to a phase 2 diet for two weeks post-weaning resulted in a higher average daily gain and body weight. MAP-fed piglets had increased villus height and decreased crypt depth, and an increased villus height to crypt depth ratio in the duodenum, jejunum, and ileum. Furthermore, MAP-fed piglets were protected against weaning-induced downregulation of tight junction protein ZO-1 and inflammation-induced increases in claudin-1. IAP is known to be downregulated post-weaning due to increased inflammatory mediators which inhibit gene expression. Therefore, we sought to determine the effects of exogenous MAP supplementation on endogenous IAP gene expression and found that dietary MAP-supplementation significantly increased IAP gene expression in the duodenum, jejunum, and ileum. Furthermore, alkaline phosphatase activity was significantly higher in the digesta and mucosa of the duodenum, jejunum, and ileum of weaned pigs eating a MAP-supplemented diet.

Another significant role of the small intestine is to act as a barrier against antigens and pathogens. The gastrointestinal tract is lined with a single layer of epithelial cells that form a selective barrier and act as the first line of defense against potentially harmful compounds and microorganisms in the intestinal lumen. Intestinal barrier dysfunction is characterized by increased intestinal permeability, or “leaky gut,” which allows harmful immunogenic agents to cross the epithelium and gain access to protected tissues and systemic circulation. This breach of the epithelial lining and subsequent translocation of luminal contents leads to increased inflammation, malabsorption, diarrhea, and potential enteric disease_{4,19,20}. The various environmental and psychological stressors on the weanling piglet contribute to the deterioration of the small intestinal barrier function, most likely through the release of stress mediators, including the aforementioned corticotrophin-releasing factor and adrenal glucocorticoids₂₁₋₂₃. Morphological changes may impair intestinal barrier function and lead to increased gut permeability, which results in increased bacterial translocation and inflammatory response. Enterocytes are joined together by tight junctions that consist of proteins which function to connect the cytoskeletons of adjacent enterocytes₂₄. Tight junctions are mainly constructed from the transmembrane protein complexes occludins and claudins, and the cytosolic protein zonula occludens. Alterations in tight junction proteins are biomarkers of increased intestinal permeability. Increased translocation of enteric pathogens, including endotoxins from Gram-negative bacteria, disrupt these proteins and increase intestinal permeability₂₅. The immature nature of the weanling piglets immune and digestive systems contributes to the duration and severity of GIT dysfunction.

Alkaline phosphatases (AP) are homodimeric enzymes that catalyze the hydrolysis of monoesters of phosphoric acid and transphosphorylation reactions. AP are naturally occurring in the mammalian body and are divided into four types: tissue non-specific AP (TNAP), placental AP (PLAP), germ cell AP (GCAP) and intestinal AP (IAP)₆₇. Intestinal alkaline phosphatase is a glycoprotein anchored in the apical membrane of the small intestine and is found in the highest levels in the duodenum, followed by the jejunum and ileum. IAP has several biological roles, including being a negative regulator of intestinal fat absorption, maintaining bicarbonate secretion and pH balance, and exerting immune-protective effects_{68,69}. Expression of IAP is dependent upon enterocyte differentiation; therefore, the enzyme is often used as a biomarker for abnormal digestive and absorptive functions in the small intestine.

Specific proteins in tight junction adhesion complexes reside in lipid rafts on the apical membrane of the intestinal epithelial cells, and during lipid absorption, IAP is located in the same area. Lipid rafts are a unique subdomain of the plasma membrane and are enriched in glycosphingolipids, cholesterol, and sphingomyelin. When Caco-2 cells, an intestinal cell line, were treated with the pro-inflammatory cytokine IFN-γ, the disruption of lipid rafts preceded intestinal permeability failure₇₀. The study also confirmed this in dextran sulfate sodium (DSS) induced colitis in mice and human ulcerative colitis patients. Treatment of another intestinal epithelial cell line, T84, with TNFα and IFN-γ resulted in altered lipid composition of lipid rafts and loss of barrier function by displacing occludins₆₅. Another study determined that these lipid rafts were released from the tips of the microvilli of intestinal epithelial cells and concluded that the shedding is one mechanism for distributing IAP activity throughout the mucosal layer of the gut₇₁. Finally, the same study also reported that both IAP expression and luminal vesicle production and shedding upregulated in the presence of Gram-negative bacteria.

The toxicity of LPS, specifically the lipid A moiety, is dependent upon its phosphorylation state₇₂. Since IAP is found in the small intestine and catalyzes the hydrolysis of phosphoric groups, and the toxicity of LPS is dependent upon the phosphorylation state, it stands to reason that IAP could dephosphorylate LPS.

The ability of IAP to dephosphorylate LPS, at physiological pH, and to reduce the toxic effects in vivo was first shown in 199773,74. A significant role of alkaline phosphatase in vivo is to detoxify bacterial LPS that is present in the intestinal lumen. High levels of IAP can control LPS-induced inflammation in two ways: by dephosphorylating LPS and decreasing TLR4 stimulation, and by preventing NF-κB translocation to the nucleus by inhibiting the phosphorylation of two critical proteins in the pathway, IκBα and RelA/p65₇₅. IAP activity has been shown to increase in the presence of Gram-negative bacteria or with LPS alone, and Gram-positive bacteria that lack LPS did not affect IAP gene expression₇₆. The efficacy of IAP to dephosphorylate LPS and reduce its toxic effects have been shown many times in experiments with IAP administered intra-peritoneal and attenuating LPS toxicity_{75,76}. Furthermore, oral administration of IAP prevented GIT tissue damage and pro-inflammatory cytokine expression that was induced by dextran sulfate sodium (DSS) in an inflammatory bowel disease (IBD) model (colitis) in mice₇₇.

During weaning, piglets go through a fasting period as their immature digestive and immune systems are maturing, and it is well known that fasting dramatically decreases IAP activity₇₈. Expression of IAP was also significantly reduced in weaned piglets compared to suckling, and this contributes to the increased occurrence of GIT pathogens₇₉. Fasting also decreases Lactobacillus populations which allow overpopulation of Gram-negative bacteria, and treatment with the probiotic Lactobacillus casei stimulated IAP activity₈₀. IAP's ability to reduce bacterial translocation and attune commensal microbiota may be an ancillary effect of pH regulation, as an alkaline microenvironment is unfavorable for the growth of pathogens₆₈.

Porcine epidermal growth factor (pEGF) is found in sow milk and contributes to postnatal gut mucosal growth and development, and a recent study showed that supplementing weaned pigs with exogenous pEGF caused an increase in gene expression and protein activity of digestive enzymes in the GIT, including IAP_{81,82}. Therefore, weaning contributes to a decrease in expression and activity of immuno-protective enzyme intestinal alkaline phosphatase, but exogenous supplementation exerts protective effects against LPS-mediated disease.

### Monophosphoryl Lipid A

LPS contains two phosphate groups in the lipid A moiety, and interactions with alkaline phosphatases cause the release of inorganic phosphate and the formation of monophosphoryl lipid A (MPLA)_{74,83}. The absence of the 1-phosphate on MPLA is believed to weaken the dimerization of TLR4/MD2, which presumably induces a structural change in the TLR4 receptor complex that alters the recruitment of adaptor proteins₈₄. MPLA has distinct signaling properties as it predominantly activates the TLR4/TRAM-TRIF pathway over the more inflammatory MyD₈₈-dependent pathway₈₅. This difference in observed effects after exposure to LPS or MPLA could be explained as an active suppression, rather than a passive loss, of pro-inflammatory activity. MPLA is classified as a TLR4 agonist, meaning it signals through TLR4/MD2 and maintains the immuno-stimulatory properties of LPS but with reduced toxicity and elicited inflammatory response. Specifically, MPLA increases antigen presentation of antigen presenting cells, like dendritic cells or macrophages, and subsequent activation of the MHC in effector T and B cells₈₆.

The gut epithelium has specialized epithelial cells called microfold cells (M cells) that transport luminal antigens and bacteria to underlying antigen presenting cells and reside in the follicle-associated epithelium surrounding Peyer's patches. Peyer's patches are aggregated lymphoid follicles and are part of the gut-associated lymphoid tissue (GALT), which consists of both aggregated and isolated lymphoid follicles₈₇. The GALT accounts for about 70% of the body's immune system and is the largest lymphoid organ. Exposing M cells to MPLA increases their ability to uptake antigens and present them to lymphocytes in the GALT, which is vital to mounting humeral and cell mediates responses₈₈. Furthermore, increasing the antigen uptake ability of one component of the GALT may increase the antigen presentation ability of the entire tissue. Due to its ability to increase antigen uptake and presentation of APC, MPLA dramatically enhances the efficacy of mucosal delivered vaccines and is currently being used as a vaccine adjuvant_{89,90}.

Current literature on the benefits of alkaline phosphatase and monophosphoryl lipid A utilizes the mammalian IAP isoform which is often derived from swine or bovine. Obtaining IAP from a mammalian host requires euthanizing the animal and scraping the mucosa of the small intestine, followed by various digestion and isolation techniques. This process requires land and money to house the animals and feed for them, produces negative environmental output, and has enzymatic variability between animals due to diet, disease state, and genetics. Furthermore, it requires large numbers of animals for a small amount of enzyme. Overall, it results in a high economic and environmental cost for a highly variable product.

Microbial enzymes are used in a variety of fields and have a large number of biotechnological applications. Bacterial hosts can be engineered to rapidly and efficiently overexpress recombinant enzymes and then cultured in large quantities to produce a substantial amount of the desired enzymes₉₁. The fast growth rate and simple requirements of microbes make them a more sustainable, economically, and environmentally friendly option. Microbial derived enzymes are rapidly becoming more popular than conventional enzymatic production methods due to their consistency, ease of optimization, regular supply, and greater catalytic activity₉₂. Recently, a microbe-derived alkaline phosphatase has been produced that purportedly can detoxify Gram-negative bacteria LPS and provide the same GIT benefits and immune-stimulatory effects as mammalian-derived IAP.

Therefore, exogenous supplementation of a microbe-derived AP could interact with the increased populations of Gram-negative bacteria associated with weaning and detoxify their LPS products and strengthen gastrointestinal barrier function. This interaction would lead to a decreased inflammatory response, decreased morphological changes, microbiome symbiosis, increased nutrient absorption and ion channel usage, and an overall decreased mortality and increased growth rate during weaning. A goal of this study was to determine the ability of microbe-derived AP (MAP) to reduce the toxic effects of LPS in vitro and in vivo, and if exogenous supplementation of MAP during the post-weaning phase would result in increased intestinal IAP expression and activity, and protect against post-weaning syndrome-induced inflammation and GIT perturbations.

The data indicates that MAP (specifically the heat tolerant AP mutant APHT, or variants thereof) may serve as a potential dietary additive to mitigate the effects of LPS- and weaning-induced inflammation on innate immune cell cytokine production, GIT permeability and morphology, tight junction protein perturbations, and downregulation of IAP gene expression and activity.

### Microbial-Derived Alkaline Phosphatase Detoxifies Lipopolysaccharide and Alters Sickness Behavior and Inflammatory Response in Weaning Pigs

Mammalian-derived intestinal alkaline phosphatase (IAP) detoxifies lipopolysaccharide (LPS) but it is not readily available for commercial application. Therefore, we sought to determine the ability of a microbial-derived alkaline phosphatase (MAP) to detoxify LPS and reduce the inflammatory response in vitro and in vivo. Compared to Salmonella enterica LPS or Escherichia coli LPS, MAP-detoxified LPS-stimulated primary alveolar macrophages had reduced transcription of pro-inflammatory cytokines TNFα, IL-6, and IL-1β, and anti-inflammatory cytokine IL-10 (P<0.001). Weaned piglets injected i.p. with MPLA or saline had no differences in serum TNFα, IL-6, IL-10, or IL-4 concentrations. LPS-injected piglets had higher serum TNFα (P=0.005) and IL-6 (P=0.006) levels than control piglets at peak-challenge, and LPS-induced TNFα remained elevated 4 hours post-injection (P=0.003). LPS injected piglets had serum IL-10 and IL-4 as compared to control (P=0.025; P=0.045) and MPLA treated pigs (P=0.006; P=0.05). LPS treatment upregulated TNFα transcript in the spleen, as compared to control (P=0.006) and (P=0.029) MPLA treated piglets. LPS treatment caused sickness behavior as measured by decreased feed intake (P=0.031) and lethargy; however, MPLA did not induce behavioral changes. Furthermore, novel findings comparing IAP and MAP, show that MAP has an increased efficacy of detoxifying LPS and ameliorating macrophage inflammatory responses in vitro. IAP-detoxified LPS-treated primary alveolar macrophages had increased gene expression of TNFα, IL-1β, and IL-10 compared to cells stimulated with MAP-detoxified LPS (P<0.001). Together these results support the efficacy of MAP to detoxify both S. enterica and E. coli LPS and reduce the toxic effects of LPS in primary cultured alveolar macrophages and in weaning piglets.

In this study, we have shown that microbial-derived alkaline phosphatase (specifically APHT) detoxifies both Salmonella and E. coli LPS in murine macrophages and primary porcine alveolar macrophages, and i.p. administration of the product MPLA (APHT) in weaning piglets resulted in significantly lower circulating pro- and anti-inflammatory cytokines, lower mRNA expression of cytokines in primary immune organs and is not potent enough to elicit sickness behavior. Novel findings indicate a bolstered ability of MAP to detoxify Gram-negative bacteria, as compared to IAP. Taken together, these present findings suggest microbial-derived AP, and MPLA could represent a safe and effective therapeutic approach to LPS-mediated disease as both prevention and treatment.

Dietary Intervention with Microbial-Derived Alkaline Phosphatase Exerts Protective Effects Against Post-Wean Syndrome in Piglets

Piglet weaning is associated with growth plateaus, inflammation, and increased endotoxin load in the gastrointestinal tract (GIT). Furthermore, weaning is characterized by perturbations in GIT enzymatic activity and morphological changes, which drive further inflammation and decrease the digestive and absorptive capacity of the small intestine, as well as overwhelm the innate immune response. Increased inflammation and bacterial load lead to displaced tight junction proteins (TJP) and inhibits the immunoprotective effects of intestinal alkaline phosphatase (IAP). Recent in vivo data has shown that microbial-derived alkaline phosphatase (MAP) is capable of detoxifying lipopolysaccharide (LPS), an endotoxin from Gram-negative bacteria such as E. coli. In the present study we focused on elucidating the effects of exogenous MAP supplementation on piglets post-wean. We found that MAP-fed piglets had a higher average daily gain (P=0.004) and increased villus height to crypt depth ratio in the duodenum, jejunum, and ileum (P<0.001). Compared to control piglets, MAP-fed piglets had increased gene expression of TJP ZO-1 in the jejunum (P<0.001) and ileum (P=0.05), and decreased gene expression of duodenal and jejunal claudin-1 (P=0.04; P=0.02). Duodenal (P=0.009), jejunal (P=0.006) and ileal (P=0.003) IAP gene expression was substantially upregulated in MAP-supplemented piglets. Finally, exogenous MAP increased alkaline phosphatase enzymatic activity in the digesta of the duodenum (P<0.001), jejunum (P=0.03) and ileum (P=0.002); and the mucosa of the duodenum (P=0.04), jejunum (P=0.002), and ileum (P=0.02). Taken together, these results indicate and provide a therapeutic intervention approach utilizing APHT to protect piglets against weaning-induced growth plateaus, disruptions against GIT tight junction proteins, and inhibition of IAP and AP activity due to increased bacterial load and inflammation during the post-weaning period.

### Materials and Methods

### Animals and Diets

Sixteen male PIC piglets from University of Illinois swine herd were naturally farrowed and weaned at 21 days of age. Piglets were kept on the premises of University of Illinois Imported Swine Research Farm and randomly divided into two treatment groups (n=8) controlling for litter of origin and body weight. Piglets were housed in four pens of four piglets each. All four pens were kept on standard phase 1 diet for one-week post-weaning to acclimate to the novel environment and piglets (Table 16). On postnatal day 28, two pens were switched to a standard phase 2 diet, and two pens were placed on a standard phase 2 diet supplemented with 4,000 IU/kg of MAP (Table 15). The experimental and control diets and fresh, clean drinking water were offered ad libitum throughout the experimental period. All piglets were maintained on control or experimental diet until postnatal day 42. Postnatal day 42, piglets were euthanized according to University of Illinois swine farm standard operating procedure as described in the AVMA Guidelines on Euthanasia, and tissues were collected for analysis. Representative segments from the duodenum, jejunum, and ileum were collected for histology. Digesta contents and mucosa were collected from duodenum, jejunum, and ileum and flash frozen for gene expression and AP activity. Weight of piglets was determined at post-natal day 21, 28, 35, and 42. Group housing and ad libitum feed prevented individual feed intake data collection.

TABLE 16
Composition of experimental diets

| Ingredient (%) | Control | MAP |
|---|---|---|
| Corn | 42.02 | 42.01 |
| Soybean meal | 31.50 | 31.49 |
| Whey | 20.00 | 19.99 |
| Fat | 3.00 | 3.00 |
| Dicalcium phosphate | 0.70 | 0.70 |
| Limestone | 1.20 | 1.20 |
| L-lysine-HCl | 0.35 | 0.35 |
| DL-methionine | 0.15 | 0.15 |
| L-threonine | 0.08 | 0.08 |
| Vitamin premix | 0.20 | 0.20 |
| Swine trace mineral premix | 0.35 | 0.35 |
| Salt-white | 0.20 | 0.20 |
| Zinc oxide | 0.25 | 0.25 |
| Microbial alkaline phosphatase (MAP) | 0.00 | 0.03 |
| Total | 100.00 | 100.00 |

### Alkaline Phosphatase Activity

AP activity was determined using a colorimetric assay kit (Abcam, USA, ab83369) according to the manufacturer's protocol. Briefly, digesta was collected from the duodenum, jejunum, and ileum and suspended in an equal volume of 0.9% saline plus protease inhibitor cocktail (11873580001; Sigma-Aldrich) at pH 8.5, centrifuged and supernatant collected for assay. Mucosal scrapings were collected with a glass slide from the duodenum, jejunum, and ileum and homogenized with equal volume 0.9% saline plus protease inhibitor cocktail at pH 8.5. The homogenized mucosa was centrifuged and pelleted and the supernatant collected for assay. AP activity was determined with respect to the release of p-nitrophenol from the p-nitrophenylphosphate (pNP) substrate. Each reaction was initiated by the addition of pNP to small intestine mucosa and digesta, and the reaction was stopped sixty minutes later with the addition of stop solution. Optical density was measured at 405 nm to quantify the amount of p-nitrophenol produced. AP activity was determined using the below equation where B equals the amount of pNP in the sample well calculated from the standard curve, ΔT is the reaction time in minutes, V is the original sample volume in the reaction well, and D is the sample dilution factor:

$$ALP Activity = ( \frac{B}{Δ T * V} ) * D \vert$$

In the present study, we found that supplementing post-weaning pigs with MAP protects against weaning-induced growth depression, villous atrophy and crypt hyperplasia, decreased IAP activity and gene expression, and disruption of tight junction proteins.

At weaning, piglets are moved from a highly digestible liquid diet to a less palatable dry diet, and as a consequence, feed intake is reduced, and the piglet has reduced nutrient absorption and utilization leading to reduced growth performance. It is essential to get pigs growing as soon as possible after weaning, and Tokach et al. (1992) reported that weight gain in the first few weeks after weaning impacts the total days to market₁₁₂. We found that weaned piglets fed a standard phase 2 diet supplemented with 4,000 IU/kg body weight MAP gained significantly more body weight during the second week of study compared to piglets fed a standard control phase 2 diet. Furthermore, during the second week on the experimental diet, piglets supplemented with MAP had a significantly higher average daily gain. Taken together, these results indicate a therapeutic approach to decreasing growth depression in weaning piglets.

MAP reported an increase in villus height in the duodenum and jejunum and a decrease in crypt depth in the duodenum, jejunum, and ileum. Furthermore, there was a significant increase in the villus height to crypt depth ratio for all segments of the small intestine. To our knowledge, this is the first study of its kind to evaluate the effects of exogenous AP supplementation on gut morphology of post-weaning piglets. Reduced intestinal inflammation from MAP detoxifying bacterial components in the lumen and increased weight gain from enhanced utilization of enteral nutrients may be responsible for the optimal villus height and crypt depth that is seen in MAP-supplemented piglets.

Compared to control phase 2 diet fed piglets, MAP-supplemented piglets had significantly higher AP activity in the digesta and mucosa of the duodenum, jejunum, and ileum. Furthermore, MAP-supplemented piglets had significantly higher IAP gene expression in the duodenum, jejunum, and ileum. Weaning-induced inflammation is marked by upregulation of pro-inflammatory mediators such as TNFα and IL-6, which have been shown to inhibit IAP expression_{116,117}. Since weaning is known to increase endotoxin load and LPS is known to upregulate TNFα and IL-6, and one of IAP's biological roles is to mitigate the inflammatory effects of LPS and protect against bacterial translocation; it is possible the increase in IAP activity and expression is due to exogenous MAP detoxifying LPS in the lumen and protecting against inflammatory mediators. AP also create an alkaline microenvironment that is unfavorable for pathogen attachment, and exogenous MAP could potentiate this alkaline environment for optimal commensal bacterial growth which would reduce the concentration of unfavorable pathogens_{118,119}. A commensal bacterial environment would protect against inflammation and downregulation of IAP activity and expression.

IAP is a marker for enterocyte differentiation, and weaning is associated with decreased feed intake, which decreases enterocyte differentiation. Since MAP-supplemented piglets had significantly higher average daily gain, the increase in IAP gene expression could be in part due to increased feed intake. Increased feed intake would increase rapamycin-dependent signaling pathways, which would stimulate the global synthesis of intracellular proteins, including IAP. Therefore, MAP-supplementation could increase IAP expression and/or activity in the digesta and mucosa through multiple mechanisms and exerts a protective role against weaning-induced decreases in IAP activity.

In the weaned piglet, downregulation of the IAP gene is associated with poor intestinal integrity₁₂₀. Intestinal permeability has long been considered to be a measure of intestinal barrier function. The intestinal barrier is regulated by a complex system of transmembrane and cytosolic proteins called tight junction proteins. The most critical components of a tight junction are transmembrane proteins occludin and the claudin family, specifically claudin-1, and the linker protein ZO-1. Occludin is an integral membrane protein having functional roles in maintaining the integrity of the tight junction₁₂₁. Claudin-1 has diverse functions depending on cell type and host but is localized to ZO-1 expression. ZO-1 is a vital intracellular tight junction protein that is essential for tight junction assembly and link the cell cytoskeleton to transmembrane tight junction proteins occludin and claudins₁₂₂. Inflammatory mediators, such as pro-inflammatory cytokine TNFα, are released following LPS-stimulation and increase intestinal permeability by disrupting expression of claudins and occludins and by altering the lipid environment of phospholipid membranes to displace tight junction proteins_{65,66} Furthermore, TNFα is thought to mediate effects on tight junctions by downregulating ZO-1 stability and concentration at the junctional surface₁₂₃. In human and mice, exogenous IAP has been shown to prevent the development of colitis, a disease marked by poor intestinal permeability_{111,124,125}. Furthermore, IAP treatment has been shown to prevent increased intestinal permeability in a mouse starvation model₁₂₆.

In the present study, we found that MAP supplementation significantly upregulated gene expression of tight junction protein ZO-1 in the jejunum and ileum, decreased claudin-1 in the duodenum and jejunum, and exerted no effect on occludin gene expression. Our results agree with Liu et al., (2016) which showed that IAP treatment preserved localization of ZO-1 and occludin proteins during inflammation. The same study also showed that IAP gene deletion in mouse embryonic fibroblasts resulted in lower levels of ZO-1, ZO-2, and occludin compared to wild-type controls₁₂₇. Zinc is an important cofactor for optimal AP activity and Zhang et al., (2019) showed that zinc supplementation alone was sufficient to enhance ZO-1 expression in weaning piglets, and zinc-supplemented piglets had a significantly higher ADG128. Taken together, these results argue that AP exerts a protective effect on ZO-1 disruption during weaning-induced anorexia and inflammation. Studies have shown a strong correlation between a decrease in ZO-1 and occludin and an increase in intestinal permeability and a decrease in trans-epithelial resistance₁₂₉. Furthermore, recent literature has shown that tight junction protein claudin-1 is increased during intestinal inflammation₁₃₀. In the present study, we found a decrease in claudin-1 in MAP fed piglets, and this may be due to decreased intestinal inflammation as compared to control fed weaned piglets. We saw no change in occludin gene expression, which is in contrast to Liu et al., (2016) which showed IAP preserved occludin expression in mouse models where colitis decreased the expression. These differences could be due to differences in species and the pathology of weaning versus colitis.

Environmental and physiological stressors during weaning lead to a decrease in feed intake and increased intestinal inflammation, which causes changes to intestinal permeability and enzymatic activity. Together, these consequences potentiate each other and drive inflammation and bacterial translocation, which can lead to growth depression and a high mortality rate during piglet weaning. Exogenous MAP supplemented during the post-weaning phase protected piglets by detoxifying LPS in the gut lumen and creating an alkaline environment unfavorable for pathogen attachment and growth. Decreased inflammation during weaning leads to increased nutrient absorption, which protects against further GIT morphological changes and anorexia-induced decreases in tight junction proteins and endogenous IAP. Furthermore, lower concentrations of endotoxins, and subsequently inflammatory mediators, due to exogenous MAP detoxifying LPS prevent further decreases in endogenous IAP and ZO-1, and increases in claudin-1. Maintaining healthy levels of TJP and IAP allows for an increased growth rate of the weaned piglet, a more robust immune system, and a protected GIT against opportunistic pathogens. Taken together, the results of these studies indicate a novel therapeutic approach to overcoming post-wean growth plateaus, enhancing small intestinal VH:CD ratios, protecting TJP expression in the small intestine during inflammation, and maintaining high levels of IAP to exert immuno-protective effects in the gastrointestinal tract.

Feeding APHT (4,000 IU/kg) of feed for 14 days (days 7 to 21 post-weaning) improved intestinal morphology in weaned pigs. Feeding APHT increased alkaline phosphatase activity in the digesta and the mucosa of weaned pigs. It also upregulated IAP gene expression in weaned pigs. Further, feeding APHT increased body weight (BW) in weaned pigs. Average body weight in kg and average daily gain in g were both increased significantly.

### REFERENCES

1. M Vente-Spreeuwenberg M A, A J Verdonk J M, Beynen A C, A Verstegen M W. Interrelationships between Gut Morphology and Faeces Consistency in Newly Weaned Piglets. Vol 77.; 2003. http://edepot.wur.nl/164825. Accessed Jan. 2, 2019.
2. Bruininx EMAM, Binnendijk G P, Van Der Peet-Schwering C M C, et al. Effect of Creep Feed Consumption on Individual Feed Intake Characteristics and Performance of Group-Housed Weanling Pigs 1,2. Vol 80.; 2002. https://pdfs.semanticscholar.org/6c59/6fb88bc84d6bbd378ec91ecd3fdae72e7df.pdf. Accessed Jan. 2, 2019.
3. Hampson D J. Alterations in piglet small intestinal structure at weaning. Res Vet Sci. 1986; 40(1):32-40. http://www.ncbi.nlm.nih.gov/pubmed/3704321. Accessed Jan. 2, 2019.
4. Boudry G, Péron V, Le Huërou-Luron I, Lallès J P, Sève B. Weaning Induces Both Transient and Long-Lasting Modifications of Absorptive, Secretory, and Barrier Properties of Piglet Intestine. J Nutr. 2004; 134(9):2256-2262. doi:10.1093/jn/134.9.2256
5. Heo J M, Opapeju F O, Pluske J R, Kim J C, Hampson D J, Nyachoti C M. Gastrointestinal health and function in weaned pigs: a review of feeding strategies to control post-weaning diarrhoea without using in-feed antimicrobial compounds. J Anim Physiol Anim Nutr (Berl). 2013; 97(2):207-237. doi:10.1111/j.1439-0396.2012.01284.x
6. Lloyd D A J, Gabe S M. Intestinal Morphology, Intestinal Regeneration and the Promise of Tissue Engineering. In: Intestinal Failure. Oxford, U K: Blackwell Publishing Ltd.; :11-19. doi:10.1002/9781405195805.ch2
7. Pluske J R, Hampson D J, Williams I H. Factors influencing the structure and function of the small intestine in the weaned pig: a review. Livest Prod Sci. 1997; 51(1-3):215-236. doi:10.1016/SO301-6226(97)00057-2
8. Hampson D J, Kidder D E. Influence of creep feeding and weaning on brush border enzyme activities in the piglet small intestine. Res Vet Sci. 1986; 40(1):24-31. http://www.ncbi.nlm.nih.gov/pubmed/3085180. Accessed Jan. 2, 2019.
9. Castillo M, Martin-Orde S M, Nofrarfas M, Manzanilla E G, Gasa J. Changes in caecal microbiota and mucosal morphology of weaned pigs. Vet Microbiol. 2007; 124(3-4):239-247. doi:10.1016/J.VETMIC.2007.04.026
10. van Beers-Schreurs H M G, Nabuurs M J A, Vellenga, L, Valk H J K der, Wensing T, Breukink H J. Weaning and the Weanling Diet Influence the Villous Height and Crypt Depth in the Small Intestine of Pigs and Alter the Concentrations of Short-Chain Fatty Acids in the Large Intestine and Blood. J Nutr. 1998; 128(6):947-953. doi:10.1093/jn/128.6.947
11. Moeser A J, Klok C Vander, Ryan K A, et al. Stress signaling pathways activated by weaning mediate intestinal dysfunction in the pig. Am J Physiol Liver Physiol. 2007; 292(1):G173-G181. doi:10.1152/ajpgi.00197.2006
12. van der Meulen J, Koopmans S J, Dekker R A, Hoogendoorn A. Increasing weaning age of piglets from 4 to 7 weeks reduces stress, increases post-weaning feed intake but does not improve intestinal functionality. animal. 2010; 4(10):1653-1661. doi:10.1017/S1751731110001011
13. Pácha J. Development of Intestinal Transport Function in Mammals. Physiol Rev. 2000; 80(4):1633-1667. doi:10.1152/physrev.2000.80.4.1633
14. Wapnir R A, Teichberg S. Regulation mechanisms of intestinal secretion: implications in nutrient absorption. J Nutr Biochem. 2002; 13(4):190-199. doi:10.1016/S0955-2863(02)00181-X
15. Nabuurs M J, Hoogendoorn A, van Zijderveld F G. Effects of weaning and enterotoxigenic Escherichia coli on net absorption in the small intestine of pigs. Res Vet Sci. 1994; 56(3):379-385. http://www.ncbi.nlm.nih.gov/pubmed/8073191. Accessed Jan. 14, 2019.
16. Miller B G, Skadhauge E. Effect of Weaning in the Pig on Ileal Ion Transport Measured in vitro. J Vet Med Ser A. 1997; 44(1-10):289-299. doi:10.1111/j.1439-0442.1997.tb01113.x
17. Osek J. Prevalence of virulence factors of Escherichia coli strains isolated from diarrheic and healthy piglets after weaning. Vet Microbiol. 1999; 68(3-4):209-217. doi:10.1016/S0378-1135(99)00109-1
18. Schierack P, Steinruck H, Kleta S, Vahjen W. Virulence factor gene profiles of Escherichia coli isolates from clinically healthy pigs. Appl Environ Microbiol. 2006; 72(10):6680-6686. doi:10.1128/AEM.02952-05
19. Livingston D H, Mosenthal A C, Deitch E A. Sepsis and multiple organ dysfunction syndrome: a clinical-mechanistic overview. New Horiz. 1995; 3(2):257-266. http://www.ncbi.nlm.nih.gov/pubmed/7583167. Accessed Jan. 14, 2019.
20. Egberts H J, Vellenga L, van Dijk J E, Mouwen J M. Intestinal permeability in piglets during transmissible gastroenteritis. Zentralbl Veterinarmed A. 1991; 38(3):157-164. http://www.ncbi.nlm.nih.gov/pubmed/1830439. Accessed Jan. 14, 2019.
21. Lambert G P. Stress-induced gastrointestinal barrier dysfunction and its inflammatory effects1. J Anim Sci. 2009; 87(suppl_14):E101-E108. doi:10.2527/jas.2008-1339
22. Taché Y, Martinez V, Million M, Rivier J. Corticotropin-Releasing Factor and the Brain-Gut Motor Response to Stress. Can J Gastroenterol. 1999; 13(suppl a):18A-25A. doi:10.1155/1999/375916
23. Meddings J B, Swain M G. Environmental stress-induced gastrointestinal permeability is mediated by endogenous glucocorticoids in the rat. Gastroenterology. 2000; 119(4):1019-1028. doi:10.1053/GAST.2000.18152
24. Mitic L L, Van Itallie C M, Anderson J M. Molecular Physiology and Pathophysiology of Tight Junctions I. Tight junction structure and function: lessons from mutant animals and proteins. Am J Physiol Liver Physiol. 2000; 279(2):G250-G254. doi:10.1152/ajpgi.2000.279.2.G250
25. Berkes J, Viswanathan V K, Savkovic S D. Intestinal epithelial responses to enteric pathogens: effects on the tight junction barrier, ion transport, and inflammation. doi:10.1136/gut.52.3.439
26. Khafipour E, Munyaka P M, Nyachoti C M, Krause D O, Rodriguez-Lecompte J C. Effect of crowding stress and Escherichia coli K88+ challenge in nursery pigs supplemented with anti-Escherichia coli K88+ probiotics1. J Anim Sci. 2014; 92(5):2017-2029. doi:10.2527/jas.2013-7043
27. Stokes C, Bailey M, Haverson K, et al. Postnatal development of intestinal immune system in piglets: implications for the process of weaning. Anim Res. 2004; 53(4):325-334. doi:10.1051/animres:2004020
28. Bailey M, Clarke C J, Wilson A D, Williams N A, Stokes C R. Depressed Potential for Interleukin-2 Production Following Early Weaning of Piglets. Vol 34.; 1992. https://ac.els-cdn.com/016524279290164L/1-s2.0-016524279290164L-main.pdf?_tid=195c56e8-3df0-468a-a12e-4894df3565da&acdnat=1547481720_f2d0ae79594db7b292dfe8e8d4554alc. Accessed Jan. 14, 2019.
29. Bailey M, Haverson K, Inman C, et al. The development of the mucosal immune system pre- and post-weaning: balancing regulatory and effector function. Proc Nutr Soc. 2005; 64(04):451-457. doi:10.1079/PNS2005452
30. Li D F, Nelssen J L, Reddy P G, et al. Transient hypersensitivity to soybean meal in the early-weaned pig. JAnim Sci. 1990; 68(6):1790-1799. http://www.ncbi.nlm.nih.gov/pubmed/2384373. Accessed Jan. 14, 2019.
31. Medzhitov R, Janeway C A. Innate immunity: impact on the adaptive immune response. Curr Opin Immunol. 1997; 9(1):4-9. doi:10.1016/SO952-7915(97)80152-5
32. Janeway C. Immunogenecity signals 1,2,3 . . . and 0. Immunol Today. 1989; 10(9):283-286. doi:10.1016/0167-5699(89)90081-9
33. Akira S, Hemmi H. Recognition of pathogen-associated molecular patterns by TLR family. Immunol Lett. 2003; 85(2):85-95. doi:10.1016/S0165-2478(02)00228-6
34. Caramalho I, Lopes-Carvalho T, Ostler D, Zelenay S, Haury M, Demengeot J. Regulatory T cells selectively express toll-like receptors and are activated by lipopolysaccharide. J Exp Med. 2003; 197(4):403-411. http://www.ncbi.nlm.nih.gov/pubmed/12591899. Accessed Feb. 25, 2019.
35. Hellman J, Loiselle P M, Tehan M M, et al. Outer membrane protein A, peptidoglycan-associated lipoprotein, and murein lipoprotein are released by Escherichia coli bacteria into serum. Infect Immun. 2000; 68(5):2566-2572. http://www.ncbi.nlm.nih.gov/pubmed/10768945. Accessed Jan. 15, 2019.
36. Tang D, Kang R, Coyne C B, Zeh H J, Lotze M T. PAMPs and DAMPs: signal Os that spur autophagy and immunity. Immunol Rev. 2012; 249(1):158-175. doi:10.1111/j.1600-065X.2012.01146.x
37. Poltorak A, He X, Smirnova I, et al. Defective LPS signaling in C3H/HeJ and C57B L/lOScCr mice: mutations in Tlr4 gene. Science. 1998; 282(5396):2085-2088. http://www.ncbi.nlm.nih.gov/pubmed/9851930. Accessed Jan. 15, 2019.
38. Schumann R R, Zweigner J. A Novel Acute-Phase Marker: Lipopolysaccharide Binding Protein (LBP). Clin Chem Lab Med. 1999; 37(3):271-274. doi:10.1515/CCLM.1999.047
39. Lamping N, Hoess A, Yu B, et al. Effects of site-directed mutagenesis of basic residues (Arg 94, Lys 95, Lys 99) of lipopolysaccharide (LPS)-binding protein on binding and transfer of LPS and subsequent immune cell activation. J Immunol. 1996; 157(10):4648-4656. http://www.ncbi.nlm.nih.gov/pubmed/8906845. Accessed Jan. 15, 2019.
40. Taylor A H, Heavner G, Nedelman M, et al. Lipopolysaccharide (LPS) neutralizing peptides reveal a lipid A binding site of LPS binding protein. J Biol Chem. 1995; 270(30):17934-17938. http://www.ncbi.nlm.nih.gov/pubmed/7543094. Accessed Jan. 15, 2019.
41. Hailman E, Lichenstein H S, Wurfel M M, et al. Lipopolysaccharide (LPS)-binding protein accelerates the binding of LPS to CD14. J Exp Med. 1994; 179(1):269-277. http://www.ncbi.nlm.nih.gov/pubmed/7505800. Accessed Jan. 15, 2019.
42. Pugin J, Schurer-Maly C C, Leturcq D, Moriarty A, Ulevitch R J, Tobias P S. Lipopolysaccharide activation of human endothelial and epithelial cells is mediated by lipopolysaccharide-binding protein and soluble CD14. Proc Natl Acad Sci USA. 1993; 90(7):2744-2748. http://www.ncbi.nlm.nih.gov/pubmed/7681988.
43. Shimazu R, Akashi S, Ogata H, et al. MD-2, a molecule that confers lipopolysaccharide responsiveness on Toll-like receptor 4. J Exp Med. 1999; 189(11):1777-1782. http://www.ncbi.nlm.nih.gov/pubmed/10359581. Accessed Jan. 15, 2019.
44. Nagai Y, Akashi S, Nagafuku M, et al. Essential role of MD-2 in LPS responsiveness and TLR4 distribution. Nat Immunol. 2002; 3(7):667-672. doi:10.1038/ni809
45. Dunne A, O'Neill L A J. The interleukin-1 receptor/Toll-like receptor superfamily: signal transduction during inflammation and host defense. Sci STKE. 2003; 2003(171):re3. doi:10.1126/stke.2003.171.re3
46. Van der Poll T, Romijn J A, Endert E, Borm J J, BUller H R, Sauerwein H P. Tumor necrosis factor mimics the metabolic response to acute infection in healthy humans. Am J Physiol. 1991; 261(4 Pt 1):E457-65. doi:10.1152/ajpendo.1991.261.4.E457
47. Dinarello C A. Interleukin-1 and interleukin-1 antagonism. Blood. 1991; 77(8):1627-1652. http://www.ncbi.nlm.nih.gov/pubmed/1826616. Accessed Jan. 15, 2019.
48. Okusawa S, Gelfand J A, Ikejima T, Connolly R J, Dinarello C A. Interleukin 1 induces a shock-like state in rabbits. Synergism with tumor necrosis factor and the effect of cyclooxygenase inhibition. J Clin Invest. 1988; 81(4):1162-1172. doi:10.1172/JCI113431
49. Tilg H, Trehu E, Atkins M B, Dinarello C A, Mier J W. Interleukin-6 (IL-6) as an anti-inflammatory cytokine: induction of circulating IL-1 receptor antagonist and soluble tumor necrosis factor receptor p55. Blood. 1994; 83(1):113-118. http://www.ncbi.nlm.nih.gov/pubmed/8274730. Accessed Jan. 29, 2019.
50. O'Garra A. The immunobiology of interleukin 4. Res Immunol. 1993; 144(8):567-568. doi:10.1016/S0923-2494(05)80003-3
51. De Waal R, Figdor C G, De Vries J E. THE IMMUNOBIOLOGY OF INTERLEUKIN 4 Effects of Interleukin 4 on Monocyte Functions: Comparison to Interleukin 13. http://hdl.handle.net/2066/27153. Accessed Jan. 29, 2019.
52. Grütz G. New insights into the molecular mechanism of interleukin-10-mediated immunosuppression. J Leukoc Biol. 2005; 77(1):3-15. doi:10.1189/jlb.0904484
53. Collins P D, Williams T J. IL-8/Neutrophil-Activating Protein 1. the Characteristics of Relationship between C5a and Proteins with Inflammation in the Rabbit in Vivo. Chemoattractant Proteins in Acute The Sequential Generation of Neutrophil.; 1991. http://www.jimmunol.org/content/146/. Accessed Feb. 19, 2019.
54. Huber A R, Kunkel S L, Todd R F, Weiss S J. Regulation of transendothelial neutrophil migration by endogenous interleukin-8. Science. 1991; 254(5028):99-102. http://www.ncbi.nlm.nih.gov/pubmed/1718038. Accessed Feb. 19, 2019.
55. Nakayama D K, Geller D A, Lowenstein C J, et al. Cytokines and Lipopolysaccharide Induce Nitric Oxide Synthase in Cultured Rat Pulmonary Artery Smooth Muscle. Am J Respir Cell Mol Biol. 1992; 7(5):471-476. doi:10.1165/ajrcmb/7.5.471
56. Bogdan C. Nitric oxide and the immune response. Nat Immunol. 2001; 2(10):907-916. doi:10.1038/ni1001-907
57. Nathan C, Shiloh M U. Reactive oxygen and nitrogen intermediates in the relationship between mammalian hosts and microbial pathogens. Proc Natl Acad Sci USA. 2000; 97(16):8841-8848. http://www.ncbi.nlm.nih.gov/pubmed/10922044. Accessed Feb. 19, 2019.
58. Bru B, Von Knethen A, Sandau K B, Ène B B. Nitric Oxide (NO): An Effector of Apoptosis. http://www.stockton-press.co.uk/cdd.
59. Universidad de Antioquia. Facultad de Ciencias Agrarias. J, Colegio de Médicos Veterinarios y de Zootecnistas de Antioquia. J, Ortiz L, Ramirez M C, Rodriguez B, López Herrera A. Revista Colombiana de Ciencias Pecuarias. Vol 24. Colvesa; 1978. http://www.scielo.org.co/scielo.php?pid=S0120-06902011000400004&script=sci_arttext&tlng=pt. Accessed Feb. 19, 2019.
60. Bauer E, Williams B A, Smidt H, Verstegen M W A, Mosenthin R. Influence of the gastrointestinal microbiota on development of the immune system in young animals. Curr Issues Intest Microbiol. 2006; 7(2):35-51. http://www.ncbi.nlm.nih.gov/pubmed/16875418. Accessed Feb. 19, 2019.
61. Garc a-Herrera J, Abad B, Rodr guez-Yoldi M J. Effect of lipopolysaccharide on D-fructose transport across rabbit jejunum. Inflamm Res. 2003; 52(4):177-184. doi:10.1007/s000110300069
62. Y U LCH, PERDUE M H. Immunologically Mediated Transport of Ions and Macromolecules. Ann N Y Acad Sci. 2006; 915(1):247-259. doi:10.1111/j.1749-6632.2000.tb05248.x
63. García-Herrera J, Marca M C, Brot-Laroche E, et al. Protein kinases, TNF-α, and proteasome contribute in the inhibition of fructose intestinal transport by sepsis in vivo. Am J Physiol Liver Physiol. 2008; 294(1):G155-G164. doi:10.1152/ajpgi.00139.2007
64. Tang Z, Liu J, Sun Z, et al. Protective effects of taurine on growth performance and intestinal epithelial barrier function in weaned piglets challenged without or with lipopolysaccharide. Anim Prod Sci. 2018; 58(11):2011. doi:10.1071/AN16249
65. Li Q, Zhang Q, Wang M, et al. Interferon-γ and tumor necrosis factor-α disrupt epithelial barrier function by altering lipid composition in membrane microdomains of tight junction. Clin Immunol. 2008; 126(1):67-80. doi:10.1016/j.clim.2007.08.017
66. Mankertz J, Tavalali S, Schmitz H, et al. Expression from the human occludin promoter is affected by tumor necrosis factor alpha and interferon gamma. J Cell Sci. 2000; 113 (Pt 11):2085-2090. http://www.ncbi.nlm.nih.gov/pubmed/10806119. Accessed Feb. 26, 2019.
67. Millán J L. Alkaline Phosphatases. Purinergic Signal. 2006; 2(2):335-341. doi:10.1007/s11302-005-5435-6
68. Mizumori M, Ham M, Guth P H, Engel E, Kaunitz J D, Akiba Y. Intestinal alkaline phosphatase regulates protective surface microclimate pH in rat duodenum. J Physiol. 2009; 587(14):3651-3663. doi:10.1113/jphysiol.2009.172270
69. Nakano T, Inoue I, Koyama I, et al. Disruption of the murine intestinal alkaline phosphatase gene Akp3 impairs lipid transcytosis and induces visceral fat accumulation and hepatic steatosis. Am J Physiol Liver Physiol. 2007; 292(5):G1439-G1449. doi:10.1152/ajpgi.00331.2006
70. Bowie R V., Donatello S, Lyes C, et al. Lipid rafts are disrupted in mildly inflamed intestinal microenvironments without overt disruption of the epithelial barrier. Am J Physiol Liver Physiol. 2012; 302(8):G781-G793. doi:10.1152/ajpgi.00002.2011
71. Shifrin D A, McConnell R E, Nambiar R, et al. Enterocyte microvillus-derived vesicles detoxify bacterial products and regulate epithelial-microbial interactions. Curr Biol. 2012; 22(7):627-631. doi:10.1016/j.cub.2012.02.022
72. JERALA R. Structural biology of the LPS recognition. Int J Med Microbiol. 2007; 297(5):353-363. doi:10.1016/j.ijmm.2007.04.001
73. Poelstra K, Bakker W W, Klok P A, Kamps J A, Hardonk M J, Meijer D K. Dephosphorylation of endotoxin by alkaline phosphatase in vivo. Am J Pathol. 1997; 151(4):1163-1169. http://www.ncbi.nlm.nih.gov/pubmed/9327750. Accessed Feb. 19, 2019.
74. Poelstra K, Bakker W W, Klok P A, Hardonk M J, Meijer D K. A physiologic function for alkaline phosphatase: endotoxin detoxification. Lab Invest. 1997; 76(3):319-327. http://www.ncbi.nlm.nih.gov/pubmed/9121115. Accessed Feb. 19, 2019.
75. Goldberg R F, Austen W G, Zhang X, et al. Intestinal alkaline phosphatase is a gut mucosal defense factor maintained by enteral nutrition. Proc Natl Acad Sci. 2008; 105(9):3551-3556. doi:10.1073/pnas.0712140105
76. Bates J M, Akerlund J, Mittge E, Guillemin K. Intestinal Alkaline Phosphatase Detoxifies Lipopolysaccharide and Prevents Inflammation in Zebrafish in Response to the Gut Microbiota. Cell HostMicrobe. 2007. doi:10.1016/j.chom.2007.10.010
77. Tuin A, Poelstra K, de Jager-Krikken A, et al. Role of alkaline phosphatase in colitis in man and rats. Gut. 2009; 58(3):379-387. doi:10.1136/gut.2007.128868
78. Bamba T, Vaja S, Murphy G M, Dowling H. Effect of Fasting and Feeding on Polyamines and Related Enzymes along the Villus:Crypt Axis. Digestion. 1990; 46(2):424-429. doi:10.1159/000200417
79. Lackeyram D, Yang C, Archbold T, Swanson K C, Fan M Z. Early Weaning Reduces Small Intestinal Alkaline Phosphatase Expression in Pigs. J Nutr. 2010; 140(3):461-468. doi:10.3945/jn.109.117267
80. Thoreux K, Balas D, Bouley C, Senegas-Balas F. Diet Supplemented with Yoghurt or Milk Fermented by &lt;i&gt;Lactobacillus casei&lt;/i&gt; DN-114 001 Stimulates Growth and Brush-Border Enzyme Activities in Mouse Small Intestine. Digestion. 1998; 59(4):349-359. doi:10.1159/000007514
81. Lee D N, Chuang Y S, Chiou H Y, Wu F Y, Yen H T, Weng C F. Oral administration recombinant porcine epidermal growth factor enhances the jejunal digestive enzyme genes expression and activity of early-weaned piglets. J Anim Physiol Anim Nutr (Berl). 2008; 92(4):463-470. doi:10.1111/j.1439-0396.2007.00735.x
82. Jaeger L A, Lamar C H, Cline T R, Cardona C J. Effect of orally administered epidermal growth factor on the jejunal mucosa of weaned pigs. Am J Vet Res. 1990; 51(3):471-474. http://www.ncbi.nlm.nih.gov/pubmed/2107778. Accessed Feb. 19, 2019.
83. Koyama I, Matsunaga T, Harada T, Hokari S, Komoda T. Alkaline phosphatases reduce toxicity of lipopolysaccharides in vivo and in vitro through dephosphorylation. Clin Biochem. 2002; 35(6):455-461. http://www.ncbi.nlm.nih.gov/pubmed/12413606. Accessed Feb. 19, 2019.
84. Park B S, Song D H, Kim H M, Choi B-S, Lee H, Lee J-O. The structural basis of lipopolysaccharide recognition by the TLR4-MD-2 complex. Nature. 2009; 458(7242):1191-1195. doi:10.1038/nature07830
85. Mata-Haro V, Cekic C, Martin M, Chilton P M, Casella C R, Mitchell T C. The Vaccine Adjuvant Monophosphoryl Lipid A as a TRIF-Biased Agonist of TLR4. Science (80-). 2007; 316(5831):1628-1632. doi:10.1126/science.1138963
86. De Becker G, Moulin V, Pajak B, et al. The adjuvant monophosphoryl lipid A increases the function of antigen-presenting cells. Int Immunol. 2000; 12(6):807-815. doi:10.1093/intimm/12.6.807
87. Neutra M R, Mantis N J, Kraehenbuhl J-P. Collaboration of epithelial cells with organized mucosal lymphoid tissues. Nat Immunol. 2001; 2(11):1004-1009. doi:10.1038/ni1101-1004
88. Gebert A, Rothk6tter H J, Pabst R. M cells in Peyer's patches of the intestine. Int Rev Cytol. 1996; 167:91-159. http://www.ncbi.nlm.nih.gov/pubmed/8768493. Accessed Feb. 25, 2019.
89. Sarti F, Perera G, Hintzen F, et al. In vivo evidence of oral vaccination with PLGA nanoparticles containing the immunostimulant monophosphoryl lipid A. Biomaterials. 2011; 32(16):4052-4057. doi:10.1016/j.biomaterials.2011.02.011
90. Childers N K, Miller K L, Tong G, et al. Adjuvant activity of monophosphoryl lipid A for nasal and oral immunization with soluble or liposome-associated antigen. Infect Immun. 2000; 68(10):5509-5516. http://www.ncbi.nlm.nih.gov/pubmed/10992447. Accessed Feb. 25, 2019.
91. Liu L, Yang H, Shin H, et al. How to achieve high-level expression of microbial enzymes. Bioengineered. 2013; 4(4):212-223. doi:10.4161/bioe.24761
92. Gurung N, Ray S, Bose S, Rai V. A Broader View: Microbial Enzymes and Their Relevance in Industries, Medicine, and Beyond. Biomed Res Int. 2013; 2013:1-18. doi:10.1155/2013/329121
93. Silverman M H, Ostro M J. Bacterial Endotoxin in Human Disease s How Advances in Understanding the Role of Gram-Negative Bacteria and Endotoxin in Infectious Diseases and Complications May Improve the Development of Diagnostic and Treatment Options.; 1998. http://citeseerx.ist.psu.edu/viewdoc/download?doi=10.1.1.360.4510&rep=rep1&type=pdf. Accessed Apr. 16, 2019.
94. Bates J M, Akerlund J, Mittge E, Guillemin K. Intestinal Alkaline Phosphatase Detoxifies Lipopolysaccharide and Prevents Inflammation in Zebrafish in Response to the Gut Microbiota. Cell Host Microbe. 2007; 2(6):371-382. doi:10.1016/j.chom.2007.10.010
95. Webel D M, Finck B N, Baker D H, Johnson R W. Time course of increased plasma cytokines, cortisol, and urea nitrogen in pigs following intraperitoneal injection of lipopolysaccharide. J Anim Sci. 1997; 75(6):1514. doi:10.2527/1997.7561514x
96. Livak K J, Schmittgen T D. Analysis of Relative Gene Expression Data Using Real-Time Quantitative PCR and the 2-ΔΔCT Method. Methods. 2001; 25(4):402-408. doi:10.1006/METH.2001.1262
97. Koch M H J, Kusumoto S, Seydel U, et al. IL-6-Inducing Capacity Influences Their Conformation and The Charge of Endotoxin Molecules.; 1998. http://www.jimmunol.org/content/161/10/5464. Accessed Apr. 5, 2019.
98. Leist M, Gantner F, Jilg S, Wendel A. Activation of the 55 kDa TNF receptor is necessary and sufficient for TNF-induced liver failure, hepatocyte apoptosis, and nitrite release. J Immunol. 1995; 154(3):1307-1316. http://www.ncbi.nlm.nih.gov/pubmed/7822799. Accessed Apr. 5, 2019.
99. Bozza F A, Salluh J I, Japiassu A M, et al. Cytokine profiles as markers of disease severity in sepsis: a multiplex analysis. Crit Care. 2007; 11(2):R49. doi:10.1186/cc5783
100. Fiorentino D F, Zlotnik A, Mosmann T R, Howard M, O'Garra A. IL-10 inhibits cytokine production by activated macrophages. J Immunol. 1991; 147(11):3815-3822. http://www.ncbi.nlm.nih.gov/pubmed/1940369. Accessed Apr. 11, 2019.
101. Cohen J. The immunopathogenesis of sepsis. Nature. 2002; 420(6917):885-891. doi:10.1038/nature01326
102. Shock Society. HBV der V van L-W M, European Shock Society., Brazilian Shock Society., International Federation of Shock Societies. Shock: Molecular, Cellular, and Systemic Pathobiological Aspects and Therapeutic Approaches: The Official Journal of the Shock Society, the European Shock Society, the Brazilian Shock Society, the International Federation of Shock Societies. Vol 18. BioMedical Press; 2002. https://insights.ovid.com/crossref/00024382-200212000-00013?isFromRelatedArticle=Y. Accessed Apr. 12, 2019.
103. Kliewer S A, Xu H E, Lambert M H, Willson T M. Peroxisome proliferator-activated receptors: from genes to physiology. Recent Prog Horm Res. 2001; 56:239-263. doi:10.1210/RP.56.1.239
104. Harvie M, Jordan T W, La Flamme A C. Differential liver protein expression during schistosomiasis. Infect Immun. 2007; 75(2):736-744. doi:10.1128/IAI.01048-06
105. Eimerbrink M J, Kranjac D, St. Laurent C, et al. Pre-treatment of C57BL6/J mice with the TLR4 agonist monophosphoryl lipid A prevents LPS-induced sickness behaviors and elevations in dorsal hippocampus interleukin-1β, independent of interleukin-4 expression. Behav Brain Res. 2016; 302:171-174. doi:10.1016/J.BBR.2016.01.012
106. Pena O M, Pistolic J, Raj D, Fjell C D, Hancock R E W. Endotoxin tolerance represents a distinctive state of alternative polarization (M2) in human mononuclear cells. J Immunol. 2011; 186(12):7243-7254. doi:10.4049/jimmunol.1001952
107. Biswas S K, Lopez-Collazo E. Endotoxin tolerance: new mechanisms, molecules and clinical significance. Trends Immunol. 2009; 30(10):475-487. doi:10.1016/J.IT.2009.07.009
108. Casella C R, Mitchell T C. Putting endotoxin to work for us: monophosphoryl lipid A as a safe and effective vaccine adjuvant. Cell Mol Life Sci. 2008; 65(20):3231-3240. doi:10.1007/s00018-008-8228-6
109. Michaud J-P, Halle M, Lampron A, et al. Toll-like receptor 4 stimulation with the detoxified ligand monophosphoryl lipid A improves Alzheimer's disease-related pathology. Proc Natl Acad Sci. 2013. doi:10.1073/pnas.1215165110
110. Alam S N, Yammine H, Moaven O, et al. Intestinal alkaline phosphatase prevents antibiotic-induced susceptibility to enteric pathogens. Ann Surg. 2014; 259(4):715-722. doi:10.1097/SLA.0b013e31828fae14
111. Kaliannan K, Hamarneh S R, Economopoulos K P, et al. Intestinal alkaline phosphatase prevents metabolic syndrome in mice. Proc Natl Acad Sci USA. 2013; 110(17):7003-7008. doi:10.1073/pnas.1220180110
112. Kats L J, Tokach M D, Goodband R D, Nelssen J L. Influence of weaning weight and growth during the first week postweaning on subsequent pig performance. 1992:15-17. https://krex.k-state.edu/dspace/handle/2097/2556. Accessed May 13, 2019.
113. Wang Y, Shan T, Xu Z, Liu J, Feng J. Effect of lactoferrin on the growth performance, intestinal morphology, and expression of PR-39 and protegrin-1 genes in weaned piglets1. J Anim Sci. 2006; 84(10):2636-2641. doi:10.2527/jas.2005-544
114. Yoon J H, Ingale S L, Kim J S, et al. Effects of dietary supplementation of antimicrobial peptide-A3 on growth performance, nutrient digestibility, intestinal and fecal microflora and intestinal morphology in weanling pigs. Anim Feed Sci Technol. 2012; 177(1-2):98-107. doi:10.1016/J.ANIFEEDSCI.2012.06.009
115. Lackeyram D, Yang C, Archbold T, Swanson K C, Fan M Z. Early Weaning Reduces Small Intestinal Alkaline Phosphatase Expression in Pigs. J Nutr. 2010; 140(3):461-468. doi:10.3945/jn.109.117267
116. Malo M S, Nasrin Alam S, Mostafa G, et al. Intestinal alkaline phosphatase preserves the normal homeostasis of gut microbiota. Gut. 2010. doi:10.1136/gut.2010.211706
117. McCracken B A, Spurlock M E, Roos M A, Zuckermann F A, Gaskins H R. Weaning Anorexia May Contribute to Local Inflammation in the Piglet Small Intestine. J Nutr. 1999; 129(3):613-619. doi:10.1093/jn/129.3.613
118. Malo M S, Alam S N, Mostafa G, et al. Intestinal alkaline phosphatase preserves the normal homeostasis of gut microbiota. Gut. 2010; 59(11):1476-1484. doi:10.1136/gut.2010.211706
119. Malo M S, Moaven O, Muhammad N, et al. Intestinal alkaline phosphatase promotes gut bacterial growth by reducing the concentration of luminal nucleotide triphosphates. Am J Physiol Liver Physiol. 2014; 306(10):G826-G838. doi:10.1152/ajpgi.00357.2013
120. Lackeyram D, Yang C, Archbold T, Swanson K C, Fan M Z. Early Weaning Reduces Small Intestinal Alkaline Phosphatase Expression in Pigs. J Nutr. 2010; 140(3):461-468. doi:10.3945/jn.109.117267
121. Furuse M, Hirase T, Itoh M, et al. Occludin: a novel integral membrane protein localizing at tight junctions. J Cell Biol. 1993; 123(6):1777-1788. doi:10.1083/jcb.123.6.1777
122. Suzuki T. Regulation of intestinal epithelial permeability by tight junctions. Cell Mol Life Sci. 2013; 70(4):631-659. doi:10.1007/s00018-012-1070-x
123. Cui W, Li L X, Sun C M, et al. Tumor necrosis factor alpha increases epithelial barrier permeability by disrupting tight junctions in Caco-2 cells. Brazilian J Med Biol Res. 2010; 43(4):330-337. doi:10.1590/S0100-879X2010007500020
124. Ramasamy S, Nguyen D D, Eston M A, et al. Intestinal alkaline phosphatase has beneficial effects in mouse models of chronic colitis. Inflamm Bowel Dis. 2011; 17(2):532-542. doi:10.1002/ibd.21377
125. Lukas M, Drastich P, Konecny M, et al. Exogenous alkaline phosphatase for the treatment of patients with moderate to severe ulcerative colitis. Inflamm Bowel Dis. 2010; 16(7):1180-1186. doi:10.1002/ibd.21161
126. Hamarneh S R, Mohamed M M R, Economopoulos K P, et al. A Novel Approach to Maintain Gut Mucosal Integrity Using an Oral Enzyme Supplement. Ann Surg. 2014; 260(4):706-715. doi:10.1097/SLA.0000000000000916
127. Liu W, Hu D, Huo H, et al. Intestinal Alkaline Phosphatase Regulates Tight Junction Protein Levels. J Am Coll Surg. 2016; 222(6):1009-1017. doi:10.1016/J.JAMCOLLSURG.2015.12.006
128. Zhang B, Guo Y. Supplemental zinc reduced intestinal permeability by enhancing occludin and zonula occludens protein-1 (ZO-1) expression in weaning piglets. 2019. doi:10.1017/S0007114509289033
129. Poritz L S, Garver K I, Green C, Fitzpatrick L, Ruggiero F, Koltun W A. Loss of the Tight Junction Protein ZO-1 in Dextran Sulfate Sodium Induced Colitis. J Surg Res. 2007; 140(1):12-19. doi:10.1016/J.JSS.2006.07.050
130. Poritz L S, Harris L R, Kelly A A, Koltun W A, Koltun W A. Increase in the tight junction protein claudin-1 in intestinal inflammation. Dig Dis Sci. 2011; 56(10):2802-2809. doi:10.1007/s10620-011-1688-9.

### Example 6

### Tolerance of APHT in Weaned Piglets

The objective of this study was to evaluate the tolerance of APHT alkaline phosphatase (also denoted Essencil HT) in weaned piglets for a period of 42 days. This was assessed through blood metabolites and clinical pathology parameters, and growth performance efficiencies. The experimental design was a randomized block design of three treatments (T2, T3 and T4) and feed control (T1). The experimental unit was the pen for growth performance and piglet for blood chemistry and hematology.

One-hundred and eighty (180) animals (domestic pig, Sus scrofa) were enrolled in the study from a single lot of weaned piglets. One-half of the selected animals (90 piglets) were intact males (M) and one-half (90 piglets) were females (F). The piglets were allocated to a total of 30 pens, with each pen housing 6 piglets (3M and 3F). Specific treatment groups were as follows in Table 17:

| | Target Dose of APHT | Treatment | No. pens | Piglets | Total |
|---|---|---|---|---|---|
| Group | (IU/kg) | Duration (days) | (replicates) | per Pen | Piglets |
| T1 | 0 | 42 | 6 | 6 | 36 |
| T2 | 12,000 | 42 | 8 | 6 | 48 |
| T3 | 20,000 | 42 | 8 | 6 | 48 |
| T4 | 200,000 | 42 | 8 | 6 | 48 |
| | | | | | 180 |

The test item was incorporated into feed (T2, T3 and T4). The study phases were as follows in Table 18:

| | Expected Body | | | |
|---|---|---|---|---|
| Dietary Study | Weight Range | | Treatment | |
| Phase | (kg) | Study Days | Groups | Feed Form |
| 1 | 7.0-15.1 | 0-21 | T1-T4 | Mash |
| 2 | 15.1-30.1 | 21-42 | T1-T4 | Mash |

Individual body weights were measured and recorded on days 0, 21 and 42. Feed was weighed and issued on an ‘as need’ basis to ensure ad libitum access. On day 21 remaining Phase 1 feed was weighed and discarded. On day 42 remaining Phase 2 feed was weighed and discarded. Blood samples were collected from 10 males and 10 females (from 180 enrolled piglets) on day 0 prior to feed issue, and from two piglets in each pen (one male and one female) on day 42. General health observations and pen fecal scores were performed daily.

### Feeding

The piglets were fed an appropriate weaner feed mix. A known amount of mash feed was offered to the piglets via the pig feeder box as needed. To measure feed intake, all feed additions were recorded for each pen when issued. At the end of each feeding phase (i.e. Days 21 and 42 of study) the residual feed left in the feeder was collected, weighed, recorded and discarded. The level of feed in each feeder was checked and additions of feed were done as required to ensure that the pigs had free access to feed.

### Nutritional Feed Additive (NFA)

### Nutritional Feed Additive of APHT (Also Denoted Essencil HT) was as Follows in Table 19:

| | |
|---|---|
| Name: | APHT (Essencil HT) alkaline phosphatase |
| Active Ingredients: | Alkaline phosphatase, approximately 35,000,000 IU/L |
| Appearance: | Off-brown liquid |
| Dose Form: | Essencil ™ HT alkaline phosphatase is a liquid to be sprayed post |
| | pelleting or to be manufactured into a diet as a premix |
| Dose Rate: | 0, 12,000, 20,000 and 200,000 IU/kg |
| Route of Administration: | Orally by being mixed in feed |
| Packaging: | 1.8 L in 2 L Nalgene Bottle |
| Storage Conditions: | 2-8° C. |
| Safety Precautions: | Appropriate Personal Protective Equipment was worn |

Treatment was well tolerated hematologically, hemostatically, and biochemically. The specific results are depicted in Tables 20-23 as follows:

TABLE 20
Hematological Variables Results

| | | | Essencil ™ HT IU/kg | | | | | |
|---|---|---|---|---|---|---|---|---|
| | | | (Treatment Group) - Day 42 | | | | | |
| | | | 0 | 12,000 | 20,000 | 200.000 | | |
| Item | Unit | Baseline | (T1) | (T2) | (T3) | (T4) | S.E.M. | P≤ |
| n | — | 20 | 12 | 16 | 16 | 16 | — | — |
| RBC | ×10¹²/L | 6.81 ± 0.15 | 6.77 | 7.16 | 7.17 | 6.80 | 0.16 | 0.15 |
| HB | g/L | 118 ± 2 | 115^{y} | 127^{x} | 122^{x,y} | 121^{x,y} | 3 | 0.07 |
| HCT | L/L | 0.377 ± 0.007 | 0.358 | 0.398 | 0.387 | 0.376 | 0.010 | 0.07 |
| MCV | fl | 55.6 ± 0.9 | 53.3 | 55.7 | 53.9 | 55.5 | 1.0 | 0.26 |
| MCH | pg | 17.5 ± 0.3 | 17.0 | 17.7 | 17.1 | 17.8 | 0.3 | 0.22 |
| MCHC | g/L | 315 ± 2 | 319 | 319 | 317 | 322 | 2 | 0.62 |
| PLT | ×10⁹/L | 716 ± 30 | 539 | 478 | 433 | 448 | 38 | 0.28 |
| WBC | ×10⁹/L | 14.8 ± 0.8 | 25.1^{a} | 18.9^{a,b} | 17.9^{b} | 17.7^{b} | 1.6 | 0.02 |

Notes:
^{a,b}Values within the same row with P ≤ 0.05 are significantly different
^{x,y}Values within the same row with 0.05 < P ≤ 1.10 are trending towards significance

TABLE 21
Differential Cell Count Results

| | | | Essencil alkaline phosphatase HT IU/kg | | | | | |
|---|---|---|---|---|---|---|---|---|
| | | | (Treatment Group) - Day 42 | | | | | |
| | | | 0 | 12,000 | 20,000 | 200,000 | | |
| Item | Unit | Baseline | (T1) | (T2) | (T3) | (T4) | S.E.M. | P≤ |
| n | — | 20 | 12 | 16 | 16 | 16 | — | — |
| NEUT | ×10⁹/L | 7.41 ± 0.54 | 6.99 | 6.71 | 6.63 | 5.93 | 0.41 | 0.33 |
| EOS | ×10⁹/L | 0.328 ± 0.036 | 0.256 | 0.310 | 0.277 | 0.184 | 0.035 | 0.08 |
| BASO | ×10⁹/L | 0.084 ± 0.009 | 0.606^{a} | 0.222^{a,b} | 0.169^{b} | 0.178^{b} | 0.096 | 0.02 |
| MONO | ×10⁹/L | 0.54 ± 0.04 | 1.09 | 0.84 | 0.82 | 0.70 | 0.10 | 0.10 |
| LYMPH | ×10⁹/L | 6.42 ± 0.51 | 16.3.^{a} | 10.6^{b} | 9.8^{b} | 10.5^{b} | 1.4 | 0.02 |

Notes:
^{a,b}Values within the same row with P ≤ 0.05 are significantly different

TABLE 22
Coagulation Variables Results

| | | | Essencil ™ HT IU/kg | | | | | |
|---|---|---|---|---|---|---|---|---|
| | | | (Treatment Group) - Day 42 | | | | | |
| | | | 0 | 12,000 | 20,000 | 200,000 | | |
| Item | Unit | Baseline | (T1) | (T2) | (T3) | (T4) | S.E.M. | P≤ |
| n | — | 19 | 12 | 15 | 16 | 16 | — | — |
| PT | sec. | 13.3 ± 0.2 | 13.9 | 13.2 | 13.3 | 14.0 | 0.3 | 0.14 |
| Fib | g/L | 2.58 ± 0.09 | 1.76 | 1.96 | 1.81 | 1.84 | 0.11 | 0.60 |
| APTT | sec | 20.8 ± 1.0 | 50.8 | 49.6 | 47.2 | 52.0 | 3.1 | 0.70 |

TABLE 23
Clinical Chemistry Results

| | | | Essencil ™ HT IU/kg | | | | | |
|---|---|---|---|---|---|---|---|---|
| | | | (Treatment Group) - Day 42 | | | | | |
| | | | 0 | 12,000 | 20,000 | 200,000 | | |
| Item | Unit | Baseline | (T1) | (T2) | (T3) | (T4) | S.E.M. | P≤ |
| n | — | 20 | 12 | 16 | 16 | 16 | — | — |
| AST | U/L | 42.2 ± 2.6 | 39.5 | 42.0 | 43.5 | 42.9 | 3.5 | 0.87 |
| ALT | U/L | 40.4 ± 2.0 | 35.6 | 34.9 | 35.1 | 32.6 | 2.1 | 0.76 |
| CK | U/L | 580 ± 56 | 1,378 | 1,491 | 1,661 | 1,687 | 358 | 0.93 |
| ALP | U/L | 573 ± 36 | 297 | 347 | 301 | 328 | 15 | 0.08 |
| BILES | μmol/L | 13.2 ± 1.8 | 17.1 | 17.3 | 15.6 | 19.8 | 4.2 | 0.91 |
| BUN | mmol/L | 3.17 ± 0.17 | 4.61 | 5.14 | 4.64 | 4.35 | 0.23 | 0.12 |
| CREAT | mg/dL | 91.7 ± 3.5 | 62.6 | 66.8 | 62.3 | 66.8 | 2.4 | 0.37 |
| TP | g/L | 53.1 ± 0.7 | 56.8 | 56.3 | 56.3 | 55.2 | 1.0 | 6.67 |
| BIL | umol/L | 6.59 ± 0.63 | 1.41^{a,b} | 1.33^{b} | 1.33^{b} | 3.02^{a} | 0.42 | 0.02 |
| ALB | g/L | 34.3 ± 0.8 | 31.6 | 31.7 | 32.3 | 31.8 | 0.9 | 0.94 |
| GLOB | g/L | 18.8 ± 0.8 | 24.7 | 24.6 | 24.5 | 23.4 | 0.8 | 0.61 |
| CHOL | mmol/L | 4.06 ± 0.41 | 1.66 | 1.44 | 1.64 | 1.77 | 0.09 | 0.09 |
| AMY | U/L | 1,424 ± 70 | 1,577 | 1,835 | 2,037 | 1,694 | 143 | 0.17 |
| GLUC* | mmol/L | 7.16 ± 0.26 | 5.98^{a,b} | 6.63^{a} | 6.33^{a} | 4.81^{b} | 0.29 | 0.0005 |
| PHOS | mmol/L | 3.00 ± 0.07 | 3.36 | 3.53 | 3.46 | 3.49 | 0.09 | 6.64 |
| Na | mmol/L | 141 ± 1 | 141 | 143 | 141 | 142 | 1 | 0.31 |
| Mg | mmol/L | 1.09 ± 0.03 | 0.96 | 1.03 | 1.00 | 1.01 | 0.03 | 0.46 |
| Cl | mmol/L | 104 ± 1 | 101 | 102 | 101 | 101 | 1 | 0.66 |
| Ca | mmol/L | 2.65 ± 0.02 | 2.73^{a,b} | 2.78^{a} | 2.69^{a,b} | 2.61^{b} | 0.04 | 0.02 |
| K | mmol/L | 4.05 ± 0.08 | 5.48 | 5.64 | 5.41 | 5.66 | 0.28 | 0.89 |

Notes:
^{a,b}Values within the same row with P ≤ 0.05 are significantly different
*The Wald test for pen as random effect for glucose resulted in a variable ratio of 1.5 ± 0.18 (S.E.), which is different from zero at P = 0.008. This means that a significant random effect of pen was detected for blood glucose.

White blood cells counts were significantly lower in T3 and T4 groups when compared to T1 control group (−28.7 & −29.5%; 25.1 vs. 17.9 & 17.7×109/L; for T1, T3 & T4 respectively; P=0.02); and basophil counts were significantly lower in T3 and T4 groups when compared to T1 group (−72.1 & −70.6%; 0.606 vs. 0.169 & 0.178 ×109/L for T1, T3 & T4 respectively; P=0.02).

Bilirubin was significantly lower in treatment groups T2 and T3 when compared to treatment group T4 (−56%; 3.02 vs. 1.33 & 1.33 umol/L for T4, T2 & T3 respectively; P=0.02); glucose was significantly higher in treatment groups T2 and T3 when compared to treatment group T4 (+37.8 & +31.6%; 4.81 vs. 6.63 & 6.33 mmol/L for T4, T2 & T3 respectively; P=0.0005) and calcium was significantly lower in treatment group T4 compared to T2 (−6.1%; 2.78 vs. 2.61 mmol/L for T2 & T4 respectively; P=0.02).

APHT alkaline phosphatase was well tolerated when included at 12,000 IU/kg, 20,000 IU/kg, or 200,000 IU/kg of feed. No negative effects of APHT were observed on cell parameters. No negative effects of APHT on blood chemistry was observed. APHT showed a significant reduction in immune cells likely indicating a lower state of systemic cell-mediated inflammation.

Post-weaning diarrhea in piglets is generally caused by over-proliferation of Escherichia coli and dysbacteriosis, which in turn has been positively correlated with crude protein levels in feed (Nyachoti et al., 2006; Opapeju et al., 2015; Chen et al., 2011).1-3 Overabundance of Gram-negative bacteria likely results in higher concentration of lipopolysaccharide (LPS) in the intestinal lumen. LPS causes intestinal inflammation, loosens tight junctions, and hence causes intestinal barrier failure.

The LPS in circulation may cause a systemic inflammatory response, which can be acute or chronic and results in loss of appetite, reduced ability to initiate protein synthesis and hence muscle growth. Intestinal alkaline phosphatase dephosphorylates LPS, reduces inflammation.

### Study 2:

A further comparable study was undertaken in weaned piglets evaluating alternative doses of the heat tolerant AP. The study was conducted for a period of 42 days. Three treatments were evaluated (0; 4,000 IU/kg (1×); 40,000 IU/kg (10×)) with 10 pigs per treatment. One-half of the selected animals (15 piglets) were intact males (M) and one-half (15 piglets) were females (F). The piglets were allocated to a total of 6 pens, 2 for each treatment. Weaning age was 21 days. There were three feeding phases: 0 to 7 days postweaning, 7-21 days postweaning, and 21-42 days postweaning. The animals were evaluated for tolerance to the AP doses. The results are depicted below in TABLE 24:

| | Alkaline phosphatase, IU/kg | | | | |
|---|---|---|---|---|---|
| Item | 0 | 4,000 | 40,000 | S.E.M. | P-value |
| Plasma ALP/U/L | 217.0 | 201.1 | 236.9 | 12.5 | 0.15 |
| AST, U/L | 34.50 | 38.56 | 35.10 | 2.00 | 0.33 |
| GGT, U/L | 62.30 | 65.44 | 53.80 | 7.55 | 0.54 |
| BUN, mg/dL | 6.40 | 6.78 | 6.80 | 0.56 | 0.85 |
| LDH, U/L | 0.80 | 0.72 | 0.83 | 0.15 | 0.88 |
| RBC, 10³/uL | 6.30 | 6.49 | 6.32 | 0.16 | 0.70 |
| Hemoglobin, g/dL | 35.0 | 36.2 | 36.1 | 0.92 | 0.60 |
| WBC, 10³/uL | 17.33 | 23.56 | 18.70 | 2.00 | 0.11 |
| Platelets, 10³/uL | 523 | 501 | 475 | 35 | 0.62 |
| Monocytes, 10³/uL | 1.19 | 1.68 | 1.39 | 0.29 | 0.50 |
| Initial BW, kg | 6.56 | 6.59 | 6.56 | 0.10 | 0.98 |
| Final BW, kg | 10.30 | 10.06 | 10.39 | 0.41 | 0.85 |

In conclusion, no adverse effect of the AP treatment was noted, even at 10× the anticipated commercial dose.

### Example 7

### Tolerance Evaluation of APHT in Poultry—Broiler Chickens

The objective of this study was to evaluate the tolerance of APHT (also denoted Essencil HT) alkaline phosphatase in broiler chickens for a period of 42 days.

The study was conducted as a randomized block design of three treatments (T2, T3 and T4) and feed control (T1). Each block consisted of four pens with each treatment group represented by one pen in the block. Mean comparison was done using two-sided Tukey test with α=0.05.

Treatment Groups are depicted below in Table 25:

| | Target Finished | | | | |
|---|---|---|---|---|---|
| | Feed Essencil ™ HT | Treatment | | Number of | Total |
| Treatment | Concentration | Duration | Number of | Birds per | Number of |
| Group ID | (IU/kg feed) | (days) | Pens | Pen | Birds |
| TG1 | 0 | 42 | 10 | 25 | 250 |
| TG2 | 12,000 | 42 | 10 | 25 | 250 |
| TG3 | 20,000 | 42 | 10 | 25 | 250 |
| TG4 | 200,000 | 42 | 10 | 25 | 250 |
| | | Total | 40 pens | | 1,000 birds |

A total of 1,000 clinically healthy day-of-hatch Ross 308 male broiler chickens were enrolled in the study. All treatments were administered in-feed according to color-coded group allocation (to maintain blinding); water and feed were available ad libitum for the duration of the study. The experimental unit was the pen for growth performance and the individual animal for blood chemistry, hematology and coagulation.

Birds were observed at least twice daily to determine their health status and the environmental condition of the pen and facility, with the exception of day 0 when only a single observation was required. Necropsies occurred for all birds removed (i.e., found dead or culled) during the study. Body weights (pen weights) were measured on days 0, 14, 28 and 42. Feed was weighed when issued and feed weigh-back occurred on day 14 (Starter feed), day 28 (Grower feed) and day 42 (Finisher feed). On days 42 and 43, blood was collected via cardiac puncture after euthanasia for analysis of clinical pathology parameters from three selected birds in each pen. Birds were euthanized via cervical dislocation.

Mortality, abnormal health observations, production performance variables (i.e., live average body weight, average daily gain, average daily feed intake, and feed to gain ratio), and clinical pathology variables (i.e., hematology, coagulation and clinical chemistry) were assessed.

### Results

Overall flock survival was 99.2% (i.e., mortality was 0.8%), with no statistically significant differences detected among treatment groups. Across all feed types/treatment groups, the Essencil™ HT concentration was within 114.5-125.1% of the target concentration. Significant overall treatment effects were detected for three out of 18 clinical chemistry variables: Magnesium (mmol/L), Phosphate (mmol/L) and Cholesterol (mmol/L). Magnesium levels differed between TG3 and TG4; however, none of the Essencil™HT treatments (i.e., TG2, TG3 and TG4) differed from control (TG1). Phosphate level was higher in TG4 compared to the control (TG1). Cholesterol differed between TG3 and TG4; however, none of the Essencil™ HT treatments (i.e., TG2, TG3 and TG4) differed from control (TG1). None of these changes had any biological relevance. There were no significant differences among treatment groups for hematology, coagulation, or performance parameters.

Feeding Essencil™ HT alkaline phosphatase up to, and including, 200,000 IU/kg was extremely well tolerated by chickens for fattening.

Details of the Essencil™ HT alkaline phosphatase used in the manufacturing of the finished feed were as follows:

TABLE 26
Nutritional Feed Additive

| | |
|---|---|
| Name: | APHT (Essencil ™ HT) alkaline phosphatase |
| Active Ingredients: | Alkaline phosphatase |
| Appearance: | Off-brown liquid |
| Dose Form: | Essencil ™ HT alkaline phosphatase was a |
| | liquid added to feed |
| Dose Rate: | 0; 12,000; 20,000 and 200,000 IU/kg of feed |
| Route of Administration: | Oral in feed |
| Packaging: | 2 L Nalgene Bottles |
| Storage Conditions: | 2° C. to 8° C. |
| Safety Precautions: | As described in the Safety Data Sheet (SDS) |

### Key Variables

Growth performance efficiency (average daily gain, average daily feed intake, gain efficiency) were calculated and evaluated for each study phase (Table 27). General health records were documented by treatment and cause of illness.

TABLE 27
Primary Continuous Variables

| List of Variables | Calculation for Each Pen |
|---|---|
| Gain Efficiency | Total Weight Gain (Days 0-42)/Total Feed Intake |
| (i.e., Gain:Feed) (Days 0-42) | (Days 0-42) |
| Feed Conversion Ratio | Total Feed Intake (Days 0-42)/Total Weight Gain |
| (Days 0-42) | (Days 0-42) |
| Average Daily Gain | Total Weight Gain (Days 0-42)/Total Treatment |
| (Days 0-42) | Phase Animal Days (Days 0-42) |
| Average Daily Feed Intake | Total Feed Intake (Days 0-42)/Total Animal Days |
| (Days 0-42); per animal basis | (Days 0-42) |
| Clinical pathology variables | Analysis of Variance (ANOVA) |

For the respective phase, the following calculations were used:
Total Animal Days (for a specified phase)=the sum of the number of days each animal in a pen was present in the pen during the specified phase. This calculation included animals that were removed from the pen during the specified phase.
Total Feed Intake (for a specified phase)=(Initial Feed Issued during the specified phase+Sum of all the Feed Issued during the specified phase)—Final Feed Weigh back in a specified phase.
Total Weight Gain (within a pen for a specified phase)=the sum of all body weights at the end of the specified phase plus the removal bodyweight for any animal removed during the specified phase minus the sum of all body weights at the beginning of the specified phase. This calculation included animals that were removed from the pen during the specified study phase.

### Clinical Pathology

Treatment was well tolerated in all treatment groups. There were no statistically significant findings associated with hematological parameters and coagulation. With regard to clinical chemistry, sodium, potassium, chloride, uric acid, calcium, amylase, glucose, ALT, ALP, total bilirubin, AST, CK, total protein, albumin and globulin were not impacted in a statistically significant manner (Table 28). Hematology was also not impacted (Table 29).

TABLE 28
Clinical Chemistry and Coagulation

| | TG1 | TG2 | TG3 | TG4 | | |
|---|---|---|---|---|---|---|
| Measurement | 0 U/kg | 12,000 U/kg | 20,000 U/kg | 200,000 U/kg | SEM | p-Value |
| Na (mmol/L) | 150.7 | 150.9 | 150.0 | 151.0 | 0.6 | 0.461 |
| K (mmol/L) | 6.78 | 6.67 | 6.95 | 6.87 | 0.14 | 0.618 |
| Cl (mmol/L) | 113.2 | 112.2 | 112.8 | 113.0 | 0.4 | 0.262 |
| UricA (umol/L) | 425.6 | 434.3 | 402.5 | 413.6 | 14.7 | 0.492 |
| Mg (mmol/L) | 0.940ab | 0.960ab | 0.907b | 0.963a | 0.029 | 0.033 |
| Ca (mmol/L) | 2.64 | 2.68 | 2.65 | 2.68 | 0.03 | 0.194 |
| PHOS (mmol/L) | 2.29b | 2.37ab | 2.32ab | 2.44a | 0.03 | 0.024 |
| AMY (U/L) | 597 | 521 | 549 | 1.636 | 521 | 0.372 |
| GLU (mmol/L) | 14.8 | 14.7 | 14.7 | 14.5 | 0.2 | 0.639 |
| ALT (U/L) | 1.77 | 1.73 | 1.80 | 1.73 | 0.18 | 0.985 |
| ALP (U/L) | 4,573 | 4,481 | 4,306 | 3,618 | 329 | 0.154 |
| TBIL (umol/L) | 1.60 | 1.78 | 1.69 | 1.76 | 0.11 | 0.265 |
| CHOL (mmol/L) | 3.35ab | 3.21ab | 3.13b | 3.43a | 0.07 | 0.025 |
| AST (U/L) | 387 | 399 | 425 | 442 | 21 | 0.372 |
| CK (U/L) | 22,389 | 23,428 | 22,718 | 27,320 | 2,712 | 0.662 |
| TP (g/L) | 27.5 | 27.4 | 27.3 | 27.7 | 0.4 | 0.886 |
| ALB (g/L) | 10.7 | 10.6 | 10.6 | 10.9 | 0.1 | 0.654 |
| GLOB (g/L) | 16.8 | 16.7 | 16.7 | 16.9 | 0.3 | 0.984 |
| FIB (g/L) | 0.75 | 0.71 | 0.71 | 0.78 | 0.03 | 0.294 |

TABLE 29
Hematology and Differential Cell Count

| | TG1 | TG2 | TG3 | TG4 | | |
|---|---|---|---|---|---|---|
| | 0 | 12,000 | 20,000 | 200,000 | | p- |
| Measurement | U/kg | U/kg | U/kg | U/kg | SEM | Value |
| RBC (×10\hat{ }12/L) | 2.03 | 2.03 | 2.09 | 2.06 | 0.07 | 0.936 |
| HB (g/L) | 80.23 | 81.80 | 79.93 | 77.97 | 1.99 | 0.416 |
| HCT (%) | 26.37 | 26.40 | 26.67 | 25.83 | 0.43 | 0.622 |
| MCV (fl) | 134.67 | 133.13 | 131.03 | 129.37 | 3.65 | 0.804 |
| MCH (pg) | 41.10 | 41.23 | 39.10 | 39.20 | 1.14 | 0.504 |
| MCHC (g/L) | 304.93 | 310.07 | 299.73 | 302.03 | 4.34 | 0.320 |
| THR (×10\hat{ }9/L) | 26.40 | 23.47 | 21.33 | 30.33 | 3.08 | 0.123 |
| WBC (×10\hat{ }9/L) | 18.08 | 16.70 | 18.40 | 20.67 | 2.08 | 0.361 |
| HETER (%) | 28.60 | 31.67 | 30.83 | 32.50 | 1.508 | 0.337 |
| LYMPH (%) | 54.73 | 51.20 | 53.17 | 49.63 | 1.338 | 0.094 |
| MONO (%) | 8.07 | 8.43 | 8.27 | 9.10 | 0.74 | 0.821 |
| EOS (%) | 3.33 | 3.97 | 3.40 | 4.11 | 0.76 | 0.634 |
| BASO (%) | 5.40 | 4.70 | 4.33 | 4.53 | 0.41 | 0.416 |
| HETER (×10\hat{ }9/L) | 5.20 | 5.27 | 5.74 | 6.85 | 0.68 | 0.225 |
| LYMPH (×10\hat{ }9/L) | 9.80 | 8.63 | 9.81 | 10.17 | 1.02 | 0.593 |
| MONO (×10\hat{ }9/L) | 1.51 | 1.34 | 1.46 | 1.84 | 0.24 | 0.310 |
| EOS (×10\hat{ }9/L) | 0.60 | 0.64 | 0.59 | 0.85 | 0.203 | 0.298 |
| BASO (×10\hat{ }9/L) | 0.99 | 0.81 | 0.80 | 0.94 | 0.133 | 0.582 |

There were no significant differences observed between the treatment groups for hematology and coagulation nor the performance parameters. The statistical changes in clinical chemistry are interpreted as having no biological relevance. Overall flock mortality was 0.8%, with no statistically significant differences among treatment groups. In conclusion, feeding APHT (Essencil™HT) alkaline phosphatase up to, and including, 200,000 IU per kg of feed was extremely well tolerated by chickens for fattening.

### REFERENCES

1. EFSA [European Food Safety Authority]. 2017. Guidance on the assessment of the safety of feed additives for target species. EFSA Journal 15(10):5021
2. EFSA [European Food Safety Authority]. 2018. Guidance on the assessment of the efficacy of feed additives. EFSA Journal 16(5):5274
3. COE [Council of Europe]. 2006. The European Convention for the Protection of Vertebrate Animals Used for Experimental and Other Scientific Purposes (ETS No. 123): Guidelines for the Accommodation and Care of Animals (Article 5 of the Convention)

### Example 8

### Quantification of Diet Type and Heat Tolerant Alkaline Phosphatase (APHT) on the Intestinal Permeability of Broilers

The purpose of this study was to develop a model to induce intestinal permeability failure in broilers. The study objectives were to quantify the effect of diet type on intestinal permeability of male broilers, and to determine the effect of exogenous alkaline phosphatase (native) on intestinal permeability and skin pigmentation of male broilers. The study followed a balanced complete block design of four treatments in a 2×2 factorial arrangement of treatments (T1, T2, T3 or T4) and used a total of 1200 male broiler chickens. Upon arrival at the study site, the chicks from the hatchery boxes were randomly allocated into pens according to the randomization schedule by selecting one to three healthy animals from multiple boxes until 30 were obtained. This procedure was repeated until 40 pens were filled. The treatment groups are outlined below in Table 30:

| | | Target Dose of | | | |
|---|---|---|---|---|---|
| | Diet | Test Article* | No. of | Animals | Total |
| Group | Viscosity | (IU/kg) | Pens | per pen | Animals |
| T1 | Low | 0 | 10 | 30 | 300 |
| T2 | High | 0 | 10 | 30 | 300 |
| T3 | Low | 10,000 | 10 | 30 | 300 |
| T4 | High | 10,000 | 10 | 30 | 300 |

*Native alkaline phosphatase, Essencil

The common starter diet was untreated. The test article Essencil alkaline phosphatase (native) (AP1) was incorporated into grower feed diets (T3 and T4 only). All diets were prepared using a standard feed preparation procedure and under the supervision of feed mill personnel. The target dose of the test article was 10,000 IU/kg. The confirmed dose was 12,133 and 12,600 IU/kg for T3 and T4 diets, respectively.

Birds were fed a common starter diet (untreated) from Day 0 to 14. From day 14, the birds were placed on grower treatment diets for the remainder of the study. Feed, whenever issued, and feed weigh back (on Days 14 and 28) was measured.

Pen (i.e. bulk) body weights were taken on Days 0, 14 and 28. Birds were observed at least once daily. Necropsies were performed on all animals euthanized or found dead during the study.

Skin pigmentation was performed on 5 birds per pen on days 14 and 27 using a Minolta gun and the L*a*b* scale.

On days 29 (Rooms 1 and 2) and 30 (Rooms 3 and 4), a lactose and mannitol solution was administered via crop gavage to 10 birds per pen. Blood was collected via cardiac puncture immediately following euthanasia, from 3 birds per pen at each of the following time points: approximately 60, 120 and 180 minutes post-gavage. The blood samples were centrifuged for the collection of serum which was analyzed for lactose and mannitol by LC-MS/MS.

The overall health of the flock was excellent and mortality was low (1.1% over the study duration).

The high-viscosity diet model resulted in lower skin pigmentation. This diet failed to enhance intestinal permeability as anticipated based on published results. This could be due to a high quality rearing environment of the test, the different structural carbohydrate concentrations of Australian wheat, barley, and rye grains compared to those used in the US, or unforeseen genetic adaptations of the birds used to common Australian grains.

Dietary pigment uptake has been suggested as an indirect measurement of intestinal permeability. A significant viscosity×Essencil interaction indicates that skin yellowness was maximized with Essencil in the low viscosity diet but lowest in the high viscosity diet. Yellow skin pigmentation is an economically important trait in many countries like Mexico, Guatemala, Spain, Italy, and others. A 7.3% increase in b* (i.e. yellowness) value using a sorghum-based (i.e. low viscosity) diet with Essencil could signify a plausible economically viable proposition for Essencil for regional broiler producers.

Intestinal permeability was quantified using two indigestible disaccharides in chickens: mannitol and lactose. A 33% reduction in the serum L/M ratio was obtain with Essencil, which suggests an improvement in intestinal permeability with enzyme inclusion.

Essencil improved intestinal permeability in vivo as indicated by a significant reduction of 33% in the lactose to mannitol area under the curve ratio in serum. This finding suggests an improvement in intestinal tight junction structure and/or function when native alkaline phosphatase was supplemented in the diet of broilers. Essencil improved (i.e. reduced) numerically Phase 2 FCR by 16.6 points and cumulative FCR by 5.8 points in the sorghum-based low viscosity diet.

Although the high viscosity diet failed to induce intestinal permeability as predicted, the inclusion of Essencil in a low viscosity diet resulted in a yellower skin pigmentation and an improvement in intestinal permeability. Additionally, Essencil in the low viscosity diet, when compared to the untreated control diet, improved FCR. Intestinal permeability was successfully quantified using two indigestible disaccharides (mannitol and lactose) in chickens.

These results indicate higher pigmentation of broilers in the low viscosity diet (i.e., similar to commercial European broiler diet) when AP was included in the diet. Absorption of pigments from the diet is considered an indirect measurement of better intestinal permeability and intestinal health. For example, the area under the curve (AUC) for serum lactose was lower in broilers consuming Alpine vs control diet (29,128 vs 45,437; P=0.0004, respectively) (data not shown). Birds do not have in their digestive tract the enzyme lactose needed to breakdown lactose into galactose and glucose. Therefore, appearance of the marker lactose in the serum of birds is a direct measurement of intestinal permeability; high serum lactose indicates poor ability of the gut to keep lactose in the lumen of the gut and indicates a higher degree of leakage into systemic circulation.

### REFERENCES

Blood J. Ingle A R, Allison N, Davies G R, Hill P G. 1991. Rapid enzymatic method for the measurement of mannitol in urine. Ann. Clin. Biochem. 28:401.
Chen J. Tellez G, Richards J D and Escobar J. 2015. Identification of potential biomarkers for gut barrier failure in broiler chickens. Front. Vet. Sc. 2:14.
Fung C P, Lin Y T, Lin J C, Chen T L, Yeh K M, Chang F Y, Chuang H C, Wu H S, Tseng C P, Sin L K. 2012. Klebsiella pneumoniae in gastrointestinal tract and pyogenic liver abscess. Energ. Infect. Dis. 18:1322.
Makrai L, Nenes C, Simon A, Ivanics E, Dudás Z, Fodor L, Glávits R. 2011. Association of Enterococcus cecorum with vertebral osteomyelitis and spondylolisthesis in broiler parent chicks. Acta Vet. Hung. 59:11.
Vicunia E A, Kuttappan V A, Tellez G, Hermandez-Velasco X, Seeber-Galarza R, Latorre J D, Faulkner O B, Wolfenden A D, Hargis B M, Bielke L R. 2015. Dose titrations of FITC-D for optimal measurement of enteric inflammation in broiler chickens, Poult. Sci. 94:1353.
Wideman R F. 2016. Bacterial chondronecrosis with osteomyelitis and lameness in broilers: a review. Poult. Sci. 95:325.
Gabor, H. 2019. Putative Metabolic Biomarkers for Gastro-Intestinal Permeability in Broiler Chickens. Honours Thesis, University of Wollongong Australia, Faculty of Science, Medicine and Health.

### Example 9

### Microbial-Derived Alkaline Phosphatase Dephosphorylates Lipopolysaccharide and Alters Sickness Behavior and Inflammatory Response in Weaned Pigs

Mammalian-derived intestinal alkaline phosphatase (IAP) dephosphorylates lipopolysaccharide (LPS) but it is not readily available for commercial application. Therefore, we sought to determine the ability of a microbial-derived alkaline phosphatase (MAP) to dephosphorylate LPS (DLPS) and reduce the inflammatory response in vitro and in vivo. Compared to untreated LPS from Salmonella enterica or Escherichia coli, MAP-dephosphorylated LPS caused lower transcription of pro-inflammatory cytokines TNF-α, IL-6, and IL-1β, and anti-inflammatory cytokine IL-10 (P<0.001) by primary porcine alveolar macrophages. Weaned piglets injected i.p. with DLPS or saline had no differences in serum TNF-α, IL-6, IL-10, or IL-4 concentrations. LPS-injected piglets had higher serum TNF-α (P=0.005) and IL-6 (P=0.006) levels than control piglets at peak-challenge, and LPS-induced TNF-α remained elevated 4 h post-injection (P=0.003). LPS injected piglets had serum IL-10 levels above control (P=0.025) and DLPS piglets (P=0.006). Serum IL-4 levels were elevated in LPS-treated pigs compared to both control (P=0.045) and DLPS (P=0.05) treated piglets. Serum levels of IL-10 and IL-4 were not different between control and DLPS pigs. LPS treatment upregulated TNF-α transcript in the spleen, as compared to control (P=0.006) and DLPS (P=0.029) treated piglets. LPS treatment caused sickness behavior as measured by decreased feed intake (P=0.031) and lethargy; however, DLPS did not induce behavioral changes. Furthermore, novel findings comparing IAP and MAP, show that MAP has an increased efficacy of dephosphorylating LPS and ameliorating macrophage inflammatory responses in vitro. IAP-dephosphorylated LPS-treated primary alveolar macrophages had increased gene expression of TNF-α, IL-1β, and IL-10 compared to cells stimulated with MAP-dephosphorylated LPS (P<0.001). Together these results support the efficacy of MAP to dephosphorylate both S. enterica and E. coli LPS and reduce the toxic effects of LPS in primary cultured alveolar macrophages and in weaning piglets.

### Example 10

### Dietary Intervention with Microbial-Derived Alkaline Phosphatase Exerts Protective Effects Against Post-Weaning Syndrome in Pigs

Piglet weaning is associated with growth plateaus, inflammation, and increased endotoxin load in the gastrointestinal tract (GIT). Furthermore, weaning is characterized by perturbations in GIT enzymatic activity and morphological changes, which drive further inflammation and decrease the digestive and absorptive capacity of the small intestine, as well as overwhelm the innate immune response. Increased inflammation and bacterial load lead to displaced tight junction proteins (TJP) and inhibits the immunoprotective effects of intestinal alkaline phosphatase (IAP). Recent in vivo data from our lab has shown that microbial-derived alkaline phosphatase (MAP) is capable to dephosphorylate lipopolysaccharide (LPS), an endotoxin from Gram-negative bacteria such as Escherichia coli and Salmonella enterica. In the present study we focused on elucidating the effects of exogenous MAP supplementation on piglets post-wean. We found that MAP-fed piglets had a higher average daily gain (P=0.004) and increased villus height to crypt depth ratio in the duodenum, jejunum, and ileum (P<0.001). Compared to control piglets, MAP-fed piglets had increased gene expression of TJP ZO-1 in the jejunum (P<0.001) and ileum (P=0.05), and decreased gene expression of claudin-1 in the duodenum (P=0.04) and jejunum (P=0.02). Duodenal (P=0.009), jejunal (P=0.006), and ileal (P=0.003) IAP gene expression was substantially upregulated in MAP-supplemented piglets. Finally, exogenous MAP increased alkaline phosphatase enzymatic activity in the digesta of the duodenum (P<0.001), jejunum (P=0.03), and ileum (P=0.002); and the mucosa of the duodenum (P=0.04), jejunum (P=0.002), and ileum (P=0.02). Taken together, these results indicate a novel therapeutic intervention approach to protect piglets against weaning-induced growth plateaus, disruptions against GIT tight junction proteins, and inhibition of IAP and AP activity due to increased bacterial load and inflammation during the post-weaning period.

### Example 11

### Alkaline Phosphatase Tolerance and Growth in Swine

Tolerance to microbial-derived alkaline phosphatase as well as growth was evaluated in pigs, particularly post-weaned piglets. A first study was undertaken in weaned piglets evaluating alternative doses of the heat tolerant AP. The study was conducted for a period of 42 days. Three treatments were evaluated (0; 4,000 IU/kg (1×); 60,000 IU/kg (15×)) with 60 pigs (half males and half females) per treatment and a total of 180 piglets. One-half of the selected animals (90 piglets) were intact males (M) and one-half (90 piglets) were females (F). The piglets were allocated to a total of 30 pens, 10 for each treatment. Initial age was unknown. Body weight initially ranged from 4.7 kg to 12.9 kg. There were two feeding phases: 0 to 21 days postweaning and 21-42 days pot weaning. The animals were evaluated for tolerance to the AP doses and growth was evaluated. Growth was assessed by evaluation of ADFI or average daily feed intake (kg), ADG or average daily growth (kg), feed conversion ratio (FCR), Gain:feed ratio (GF), and Final BW body weight (kg). Feed conversion ratio (FCR) is the net feed consumption of livestock unit weight gain. A reduction of FCR shows a positive effect on growth given the amount of food consumed. GF measures weight gain based on food consumption. For example, for an animal that consumes 8 pounds of feed (on a dry matter basis) and puts on 1 pound of body weight gain, its F:G would be 8.0 (8 lb÷1 lb) while its G:F would be 0.125 (1 lb÷8 lb). This is another measure of evaluating the efficacy of AP HT similar to FCR. The administered AP improved FCR (12.8 points) and GF (increased 6.4%). The results are depicted below in TABLE 31:

| | Alkaline phosphatase, IU/kg | | | | |
|---|---|---|---|---|---|
| Item | 0 | 4,000 | 60,000 | S.E.M. | P-value |
| Plasma ALP/U/L | 234.3 | 267.2 | 263.7 | 15.8 | 0.29 |
| AST, U/L | 62.4 | 55.9 | 54.9 | 7.5 | 0.74 |
| GGT, U/L | 36.3 | 31.8 | 35.7 | 2.0 | 0.25 |
| BUN, mg/dL | 4.00 | 3.65 | 3.90 | 0.22 | 0.52 |
| RBC, 10³/uL | 7.01 | 6.99 | 7.17 | 0.10 | 0.40 |
| Hemoglobin, g/dL | 123.1 | 123.7 | 127.1 | 1.6 | 0.18 |
| WBC, 10³/uL | 18.6 | 16.5 | 18.8 | 1.0 | 0.24 |
| Platelets, 10³/uL | 486 | 419 | 446 | 33 | 0.36 |
| Monocytes, 10³/uL | 1.00 | 0.85 | 0.89 | 0.08 | 0.39 |
| Initial BW, kg | 8.26 | 8.26 | 8.23 | 0.22 | 0.99 |
| ADFI, kg | 0.941 | 0.962 | 0.953 | 0.027 | 0.86 |
| ADG, kg | 0.481 | 0.509 | 0.497 | 0.017 | 0.51 |
| FCR | 2.006 | 1.878 | 1.924 | 0.026 | 0.003 |
| GF | 0.502 | 0.534 | 0.523 | 0.007 | 0.003 |
| Final BW, kg | 28.51 | 29.74 | 28.91 | 0.81 | 0.56 |

In a further study, growth in swine was evaluated, including a CP challenge. The study was conducted for a period of 42 days. Three treatments were evaluated (0; 4,000 IU/kg (1×); 8,000 IU/kg (2×)) with 60 pigs (half males and half females) per treatment and a total of 180 piglets. One-half of the selected animals (90 piglets) were intact males (M) and one-half (90 piglets) were females (F). The piglets were allocated to a total of 30 pens, 10 for each treatment. Weaning age was 21 days. The animals were of a uniform age and body weight (BW). ZnO was administered as a prophylactic at 2,500 ppm ZnO for diarrhea prevention. Pulmotil was administered for respiratory disease prevention. The gut challenge was a sharp withdrawal of prophylactics and high CP diet. There were three feeding phases: 0 to 7 days postweaning (ZnO, Pulmotil, 21.7% CP), 7 to 14 days postweaning (25.7% CP, no ZnO, no Pulmotil), and 21 to 42 days potweaning (23.6% CP, no ZnO, no Pulmotil). Growth was assessed by evaluation of ADFI or average daily feed intake (kg), ADG or average daily growth (kg), FCR, GF (Growth:Food ratio), and Final BW body weight (kg). The administered AP improved FCR (5.2 points) and GF (increased 3.4%). The results are depicted below in TABLE 32:

| | Alkaline phosphatase, IU/kg | | | | |
|---|---|---|---|---|---|
| Item | 0 | 4,000 | 8,000 | S.E.M. | P-value |
| Initial BW, kg | 5.97 | 6.06 | 5.90 | 0.10 | 0.57 |
| ADFI, kg | 0.705 | 0.710 | 0.704 | 0.012 | 0.94 |
| ADG, kg | 0.413 | 0.452 | 0.435 | 0.010 | 0.31 |
| FCR | 1.633 | 1.581 | 1.637 | 0.015 | 0.01 |
| GF | 0.614 | 0.635 | 0.615 | 0.006 | 0.01 |
| Final BW, kg | 24.08 | 24.83 | 28.16 | 0.48 | 0.49 |

### Example 12

### Alkaline Phosphatase Dose Response and Growth Performance Evaluations

Dose titration responses were evaluated in swine using a nursery pig model. Dose increment spacing was achieved using a doubling effect. Weaned pigs (131 weaned pigs at about 19 days old start age) were evaluated in a study over 28 days with 2 weeks per diet phase in 2 phases. Four (4) treatments were assessed, 7 replicates of each: Control (0 IU/kg), 3,500 IU/kg, 7,000 IU/kg, and 14,000 IU/kg. The overall results are depicted below in TABLE 33:

TABLE 33
OVERALL RESULTS

| | | Enzyme Units IU/kg | | | | |
|---|---|---|---|---|---|---|
| | Control | 3,500 | 7,000 | 14,000 | MSE | P, Diet |
| Start BW, lb | 18.53 | 18.61 | 18.73 | 18.87 | 0.66 | 0.79 |
| End BW, lb | 34.40 | 42.47 | 40.16 | 41.56 | 3.89 | 0.004 |

While the initial start BW was equivalent, the overall results show improved and higher end BW in all of the enzyme administered animals, with the 3,500 IU/kg animals demonstrating the highest end body weight (BW).

An additional study was undertaken to investigate growth comparisons and gut ecology. Weaned pigs (48 weaned pigs at about 29 days old start age; start BW 8.45±0.38 kg) were evaluated in a study over 16 days. Treatments were: Control (no enzyme), 0.1 MU/kg mannanases, 0.06 MU/kg mannanases, 0.1 Mu/kg mannanase+0.077 MU/kg glucanase, and 9,240 Mu/kg alkaline phosphatase (AP). The overall growth results are depicted below in TABLE 34:

TABLE 34
OVERALL RESULTS

| | | Enzyme Units IU/kg | | | | | |
|---|---|---|---|---|---|---|---|
| | | | | 0.10 + | | | |
| | | 0.1 | 0.06 | 0.08 | 9,240 | | P, |
| | Control | MU/kg | MU/kg | MU/kg | IU/kg | MSE | Diet |
| Start | 18.75 | 18.48 | 18.23 | 18.48 | 18.30 | 0.244 | 0.56 |
| BW, lb | | | | | | | |
| End | 36.83 | 36.32 | 34.27 | 38.25 | 37.07 | 1.094 | 0.15 |
| BW, lb | | | | | | | |
| ADG | 1.13 | 1.12 | 1.00 | 1.24 | 1.17 | 0.068 | 0.09 |
| ADFI | 1.56 | 1.50 | 1.42 | 1.75 | 1.62 | 0.087 | 0.08 |
| G:F | 0.723 | 0.745 | 0.701 | 0.710 | 0.724 | 0.024 | 0.24 |

Bacterial enumeration evaluations were conducted. In a first assessment, the concentrations of bacterial populations in the ileum were studied. The effects of in-feed enzymes on ileal and cecal microbial populations of nursery pigs have been previously evaluated and reported (Jang, J-C et al (2020) Animals 10,703; doi:10.3390/ani10040703; Perty, A L et al (2021) PLoS ONE 16(1):e0246144). In the ileum, anaerobes, coliforms, and lactobacilli were not affected by the dietary supplementation of enzymes. However, 0.06 MU/kg mannanases increased ileal aerobes (P<0.05), whereas 0.1 MU/kg mannanases increased Bifidobacterium spp. and Enterococcus spp. (P<0.05). Moreover, 0.1 MU/kg mannanase +0.077 MU/kg glucanase and also the AP reduced ileal concentrations of E. coli, and increased Bifidobacterium spp. (P<0.05). In the cecum, no effects were observed on aerobes, anaerobes, lactobacilli, and Bifidobacterium spp. However, 0.1 MU/kg mannanases increased concentrations of Enterococcus spp. (P<0.05), whereas AP reduced concentrations of coliforms and E. coli (P<0.05).

Other studies were conducted to investigate growth performance. AP administration improved Jejunal active glucose transport, Jejunal active phosphate transport, and Jejunal active chloride secretion (data not shown). Two possible roles of exogenous alkaline phosphatase (AP) enzyme in the pig were investigated: improving dietary phosphate availability and reducing gut inflammatory response due to recognition of dietary LPS by GIT innate immune system, resulting in improved nutrient utilization. The study data indicates that the addition of AP enzyme to the negative control (NC) diet increased luminal phosphate resulting in decreased active phosphate absorption, which was significantly different from the NC when the NC diet included both AP enzyme and phosphorus. The addition of alkaline phosphatase by itself did not result in full recovery from the reduction in growth performance of the negative control diet.

When AP was combined with phosphorus, pig performance was equal to the positive control. This improvement in gain and efficiency is likely related to reduced GIT immune stimulation, increasing metabolizable energy available for growth similar to the 100 kcal/kg reduction used in this study. Active glucose absorption, estimated based on changes in short circuit current was 9.78, 18.55, 23.89 and 25.05 μA/cm² for the NC, PC, NC+AP+P, and NC+AP, respectively (P=0.39, pooled SEM=17). Active phosphate absorption, based on changes in short circuit current following phosphate addition to the serosal chamber, was highest (P<0.05) for pigs fed the NC diet. The addition of AP to the NC diet resulted in a 26% reduction in active phosphate absorption (P<0.05). Pigs fed the positive control PC diet or the NC+AP+P diet had further reductions in active phosphate absorption of 53 and 64%, respectively, relative to the NC.

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A heat tolerant bacterial alkaline phosphatase enzyme polypeptide, said polypeptide comprising:
(a) the polypeptide
(SEQ ID NO: 2)
ESSNGQSKNLIVLIGDGMGPAQVSAARYFQQHX₁NNINSLNLDPYYVGQATTYADRGEDGG
HIVSGIX₂TSSAX₃AGTAX₄ATGNKTX₅NAAISVSNEDVSRPFASVX₆EAAELSGKSTGLVTTARI
THATPAVYX₇SHVRX₈RDNENAIAFQYLDSGIDVLLGGGESFFVTX₉EEKGKRNDKNLLPEFE
AKGYKVVKX₁₀GQSLKSLSAKDAKVLGLFGGSHIX₁₁YVPDRSDETPSLAEMTSKALEILSTNE
NGFAIMIEGGRIDHAX₁₂HANDFPTMVQEX₁₃LX₁₄FDEAFKVAIDFAKKDGNTSVVVTADHET
GGLSLX₁₅RDNIYELNVDLWNKQKNSSESLVSALNEAKTIADVKKIVSDNTWITDLTNEEAQY
ILDGDGSSYKREGGYNAVISKRLLVGWSGHGHSAVDVGVWAYGPIADKVKGQIDNTRIATA
SAEVLGVDLKKATADLQSKYLYPKFKINRNKEVLFPAKPLAEALGGKYQAANGTATISGMS
GTITVDLNAKKAKLSGNSSX₁₆ITIDVDNDVLYLPLTAFSQITGQTLKWDALSERIMLK
wherein:
X₁ is I or L,
X₂ is I,
X₃ is A,
X₄ is I,
X₅ is N,
X₆ is H or F,
X₇ is G,
X₈ is N,
X₉ is N,
X₁₀ is N,
X₁₁ is P,
X₁₂ is A,
X₁₃ is M, V, T or E,
X₁₄ is A or E,
X₁₅ is G or A, and
X₁₆ is E;
(b) the polypeptide:
(SEQ ID NO: 3)
ESSNGQSKNLIVLIGDGMGPAQVSAARYFQQHINNINSLNLDPYYVGQATTYADRGEDGGHI
VSGIVTSSASAGTALATGNKTYNAAISVSNEDVSRPFASVLEAAELSGKSTGLVTTARITHAT
PAVYASHVRNRDNENAIAFQYLDSGIDVLLGGGESFFVTNEEKGKRNDKNLLPEFEAKGYK
VVKTGQSLKSLSAKDAKVLGLFGGSHIAYVPDRSDETPSLAEMTSKALEILSTNENGFAIMIE
GGRIDHAGHANDFPTMVQEALDFDEAFKVAIDFAKKDGNTSVVVTADHETGGLSLSRDNIY
ELNVDLWNKQKNSSESLVSALNEAKTIADVKKIVSDNTWITDLTNEEAQYILDGDGSSYKRE
GGYNAVISKRLLVGWSGHGHSAVDVGVWAYGPIADKVKGQIDNTRIATASAEVLGVDLKK
ATADLQSKYLYPKFKINRNKEVLFPAKPLAEALGGKYQAANGTATISGMSGTITVDLNAKK
AKLSGNSSSITIDVDNDVLYLPLTAFSQITGQTLKWDALSERIMLK;
or
(c) a variant of (a) or (b), wherein the variant of the alkaline phosphatase enzyme polypeptides retains phosphatase activity which is not significantly different from heat tolerant mutant enzyme SEQ ID NO:1.

2. The heat tolerant bacterial alkaline phosphatase enzyme polypeptide of claim 1, denoted APHT, said polypeptide comprising:
(SEQ ID NO: 1)
ESSNGQSKNLIVLIGDGMGPAQVSAARYFQQHINNINSLNLDPYYVGQATTYADRGEDGGHIVSGIITSS
AAAGTAIATGNKTNNAAISVSNEDVSRPFASVHEAAELSGKSTGLVTTARITHATPAVYGSHVRNRDNEN
AIAFQYLDSGIDVLLGGGESFFVTNEEKGKRNDKNLLPEFEAKGYKVVKNGQSLKSLSAKDAKVLGLFGG
SHIPYVPDRSDETPSLAEMTSKALEILSTNENGFAIMIEGGRIDHAAHANDFPTMVQEMLAFDEAFKVAI
DFAKKDGNTSVVVTADHETGGLSLGRDNIYELNVDLWNKQKNSSESLVSALNEAKTIADVKKIVSDNTWI
TDLTNEEAQYILDGDGSSYKREGGYNAVISKRLLVGWSGHGHSAVDVGVWAYGPIADKVKGQIDNTRIAT
ASAEVLGVDLKKATADLQSKYLYPKFKINRNKEVLFPAKPLAEALGGKYQAANGTATISGMSGTITVDLN
AKKAKLSGNSSEITIDVDNDVLYLPLTAFSQITGQTLKWDALSERIMLK.

3. The heat tolerant bacterial alkaline phosphatase enzyme polypeptide of claim 1, said polypeptide comprising:
(SEQ ID NO: 3)
ESSNGQSKNLIVLIGDGMGPAQVSAARYFQQHINNINSLNLDPYYVGQATTYADRGEDGGHIVS
GIVTSSASAGTALATGNKTYNAAISVSNEDVSRPFASVLEAAELSGKSTGLVTTARITHATPAVY
ASHVRNRDNENAIAFQYLDSGIDVLLGGGESFFVTNEEKGKRNDKNLLPEFEAKGYKVVKTGQS
LKSLSAKDAKVLGLFGGSHIAYVPDRSDETPSLAEMTSKALEILSTNENGFAIMIEGGRIDHAGHA
NDFPTMVQEALDFDEAFKVAIDFAKKDGNTSVVVTADHETGGLSLSRDNIYELNVDLWNKQKN
SSESLVSALNEAKTIADVKKIVSDNTWITDLTNEEAQYILDGDGSSYKREGGYNAVISKRLLVGW
SGHGHSAVDVGVWAYGPIADKVKGQIDNTRIATASAEVLGVDLKKATADLQSKYLYPKFKINR
NKEVLFPAKPLAEALGGKYQAANGTATISGMSGTITVDLNAKKAKLSGNSSSITIDVDNDVLYLP
LTAFSQITGQTLKWDALSERIMLK

4. The heat tolerant bacterial alkaline phosphatase enzyme polypeptide of claim 1, said polypeptide comprising the polypeptide of any of SEQ ID NOs: 18-73.

5. The alkaline phosphatase enzyme polypeptide of claim 1, further comprising an N terminal leader sequence.

6. The alkaline phosphatase enzyme polypeptide of claim 5, wherein the N terminal leader sequence is a sequence of 2 to 10 amino acids and wherein the leader sequence does not alter the heat tolerance or the enzymatic activity of the phosphatase.

7. The alkaline phosphatase enzyme polypeptide of claim 5, wherein the N terminal leader sequence is selected from SEQ ID NO: 10, 13, 16 and 17.

8. A feed additive or feed composition comprising the mutant alkaline phosphatase enzyme polypeptide of claim 1.

9. The feed additive or feed composition of claim 8, comprising the mutant alkaline phosphatase enzyme polypeptide of SEQ ID NO: 1, 2, 3 or 18-73.

10. The feed additive or feed composition of claim 8, comprising the mutant alkaline phosphatase enzyme polypeptide of SEQ ID NO: 2 or of SEQ ID NO: 1.

11. (canceled)

12. The feed additive or feed composition of claim 8, comprising the mutant alkaline phosphatase enzyme polypeptide of SEQ ID NO: 1, 2, 3 or 18-73 in an amount of 4,000 IU/kg, 10,000 IU/kg, 12,000 IU/kg, 20,000 IU/kg, 40,000 IU/kg, up to 40,000 IU/kg, 100,000 IU/kg, or up to 200,000 IU/kg.

13. (canceled)

14. A composition comprising the mutant alkaline phosphatase enzyme polypeptide of claim 1 and an additive or diluent or carrier.

15. The additive or composition of claim 8, further comprising an anti-inflammatory agent, molecule or cytokine or further comprising an anti-bacterial and/or an anti-infective agent.

16. (canceled)

17. A method for reducing bacterial lipopolysaccharides (LPS) in the intestine, reducing inflammation particularly bacterial-mediated inflammation, reducing production of pro-inflammatory cytokines, particularly in the intestine, improving intestinal permeability markers, and/or improving or resulting in more efficient nutrient utilization in an animal, comprising administering to said animal the mutant alkaline phosphatase enzyme polypeptide of claim 1.

18. The method of claim 17, wherein production or expression of pro-inflammatory cytokine TNFα and/or of IL-6 is reduced in the intestine of an animal.

19. The method of claim 17, comprising administering to said animal the mutant alkaline phosphatase enzyme polypeptide of SEQ ID NO: 1, 2, 3 or 18-73.

20. The method of claim 17, comprising administering to said animal the mutant alkaline phosphatase enzyme polypeptide of SEQ ID NO: 2 or of SEQ ID NO: 1.

21. (canceled)

22. (canceled)

23. (canceled)

24. (canceled)

25. (canceled)

26. A method for increasing intestinal alkaline phosphatase production or activity in an animal, for reducing bacterial lipopolysaccharides (LPS) in the intestine, for reducing production of pro-inflammatory cytokines, or for reducing intestinal inflammation in an animal comprising administering to said animal the mutant alkaline phosphatase enzyme polypeptide of claim 1.

27. The method of claim 26, comprising administering to said animal the mutant alkaline phosphatase enzyme polypeptide of SEQ ID NO: 1, 2, 3 or 18-73.

28. The method of claim 26, comprising administering to said animal the mutant alkaline phosphatase enzyme polypeptide of SEQ ID NO: 2 or of SEQ ID NO: 1.

29. (canceled)

30. A method for increasing intestinal alkaline phosphatase production or activity in an animal, for reducing bacterial lipopolysaccharides (LPS) in the intestine, for reducing production of pro-inflammatory cytokines, or for reducing intestinal inflammation in an animal comprising administering to said animal the additive or composition of claim 8.

31. The method of claim 30, comprising administering to said animal the mutant alkaline phosphatase enzyme polypeptide of SEQ ID NO: 1, 2, 3 or 18-73.

32. The method of claim 30, comprising administering to said animal the mutant alkaline phosphatase enzyme polypeptide of SEQ ID NO: 2 or SEQ ID NO: 1.

33. (canceled)
